(19) United States
(12) Patent Application Publication
(10) Pub. No.: US 2024/0408232 A1
(43) Pub. Date: Dec. 12, 2024

(54) METHOD FOR NARROWING EDITING WINDOW OF BASE EDITOR, BASE EDITOR, AND USE

(71) Applicant: TIANJIN INSTITUTE OF INDUSTRIAL BIOTECHNOLOGY, CHINESE ACADEMY OF SCIENCES, Tianjin (CN)

(72) Inventors: Xueli Zhang, Tianjin (CN); Changhao Bi, Tianjin (CN); Dongdong Zhao, Tianjin (CN); Guo Jiang, Tianjin (CN); Xuxu Chen, Tianjin (CN)

(21) Appl. No.: 18/292,680

(22) PCT Filed: Jul. 26, 2022
(86) PCT No.: PCT/CN2022/107988

§ 371 Date: Jan. 26, 2024

(30) Foreign Application Priority Data
Jul. 30, 2021 (CN) 202110873929.5

(51) Int. Cl. A61K 48/00; C12N 9/22; C12N 9/78; C12N 15/10; C12N 15/11; C12N 15/85

(52) U.S. Cl. CPC A61K 48/005; C12N 9/22; C12N 9/78; C12N 15/102; C12N 15/111; C12N 15/85; C12N 2310/20

(57) ABSTRACT

Provided are a gRNA mutant and the use thereof, and further provided are a method for constructing same and a base editor containing same. The gRNA mutant is used in the base editor, and can universally reduce a base editing window, thereby improving the specificity of gene editing, and achieving specific editing on one base.

This is the U.S. National Phase Application under 35 U.S.C. § 371 of International Patent Application No. PCT/CN2022/107988 filed Jul. 26, 2022, which claims the benefit of Chinese Patent Application No. 202110873929.5, filed on Jul. 30, 2021 with the Chinese Patent Office, entitled “METHOD FOR NARROWING EDITING WINDOW OF BASE EDITOR, BASE EDITOR, AND USE”, both of them are incorporated by reference herein.

### SEQUENCE LISTING

This application contains a Sequence Listing that has been submitted electronically as an XML file named “54397-0009US1_SL_ST26.XML.” The XML file, created on Jul. 16, 2024, is 272,296 bytes in size. The material in the XML file is hereby incorporated by reference in its entirety.

### TECHNICAL FIELD

The present disclosure belongs to the field of biotechnology and gene editing technology, and specifically relates to a method for constructing a gRNA mutant, a gRNA mutant, a method for narrowing an editing window of a base editor, a base editor, an isolated polynucleotide, a recombinant expression vector, a recombinant host cell, a base editor, a composition, and use as well as a method for gene editing in a cell or subject and a method for treating or preventing a disease.

### BACKGROUND

CRISPR/Cas (clustered regularly interspaced short palindromic repeats/CRISPR-associated) system is a technique for a specific DNA modification of a targeted gene by an RNA-guided Cas nuclease, which is an adaptive immune defense mechanism evolved from bacteria and archaea in response to constant attacks by phages and foreign plasmids. Since its discovery, the CRISPR/Cas systems have been successfully applied to gene editing in multiple species and have been extensively used in the field of gene editing.

CRISPR/Cas systems discovered so far can be sorted into two classes (Class 1 and Class 2), and further sorted into six types (Type I to Type VI). Of these, Class 1 systems include Type I, Type III, and Type IV, and Class 2 systems include Type II, Type V, and Type VI. In the commonly used CRISPR/Cas systems, guide RNA (gRNA) guides the Cas protein to perform precise cleavages at the targeted sites of the genome, resulting in DNA double strand breaks (DSBs), and the host cell repairs the DSBs through its own non-homologous end-joining (NHEJ) or based on the homologous end recombination repair (HDR), but it is difficult to realize specific editing of a single base. Due to many uncertainties about DNA double strand breaks, HDR occurs with a very low probability, and NHEJ causes random insertions or deletions of bases. Consequently, conventional CRISPR/Cas techniques have certain disadvantages in gene editing of a single base.

The advent of base editors (BEs) remedies the defects of conventional CRISPR/Cas techniques in single-base editing. Without the occurrence of DNA double strand breaks and the participation of a donor DNA, the base editor may achieve precise point mutations at target sites and therefore holds a great application prospect in the treatment of genetic diseases caused by gene mutations. Available base editors mainly include: a cytosine base editor (CBE), which may convert the cytidylate within an editing window of the target sequence into the thymidylate (C>T): an adenine base editor (ABE), which may convert the adenylate within an editing window into the guanylate (A>G); and a novel glycosylase base editor (GBE), which may edit cytidylate into adenylate in Escherichia coli or specifically edit cytidylate into guanylate in mammalian cells.

At present, except for GBE that can precisely edit the C6 site in mammalian cells, the CRISPR/Cas-based base editors all have an editing window involving multiple nucleotides. For example, the editing window of typical CBE involves 4 or 5 bases, and the editing window of ABE also involves up to 4 bases, in which all Cs or As within the editing window may be edited. However, about half of known pathogenic mutations are point mutations (also referred to as single nucleotide polymorphism SNP). For correction of pathogenic point mutations, it is often necessary to precisely correct one base, and additional base modification(s) would cause side effects instead. It is thus particularly necessary to narrow the editing windows of CBE and ABE, or even enable the editing windows to be accurate to one base.

However, there is still a lack of a universal method for narrowing the editing window of bases now, which enables the editing windows of CBE and ABE to be accurate to one base.

In terms of CBE, the base editing window of CBE may be narrowed to some extent by selecting cytosine deaminases from different sources, different CRISPR systems, different linkers, and mutations in cytosine deaminases. In terms of ABE, no other adenine deaminases than the evolved adenine deaminases derived from bacteria have been found to be effective for A-to-G mutations after fused to the CRISPR systems. There is a lack of an effective method for narrowing the editing window of ABE. To date, there is no universal method for narrowing an editing window of a base editor, or a method that enables the editing windows of CBE and ABE to be accurate to one base, which greatly restricts the application of the base editing systems in precise base editing.

### SUMMARY

### Technical Problem

In view of the problems existing in the prior art, for example, the lack of a universal method for narrowing the editing window of the base editor and the difficulty of CBE or ABE in achieving the precise editing of a single base, the present disclosure provides a method for constructing a gRNA mutant, in which the gRNA mutant is obtained by mutating the guide sequence region in the gRNA. The gRNA mutant, when applied to the base editor, is capable of universally narrowing the base editing window and realizing the precise editing of one base.

### Solution to Problem

In the first aspect, the present disclosure provides a method for constructing a gRNA mutant, wherein the method comprises:
a mutation step: mutating a guide sequence region in a gRNA that is hybridized with a target sequence of a nucleic acid of interest, such that a substitution, deletion or insertion of one or more bases occurs at one or more positions of the guide sequence region, to form a mutation sequence region containing a mutated nucleotide; and
a screening step: screening a mutant with a narrowed editing window for a base editor as compared to an unmutated gRNA, to obtain the gRNA mutant.

In some embodiments, the screening step in the method according to the present disclosure comprises:
screening the mutant with an editing window for the base editor being a single base, to obtain the gRNA mutant.

In some embodiments, in the method according to the present disclosure, the guide sequence region has a first end proximal to a PAM sequence of the nucleic acid of interest and a second end distal to the PAM sequence; and
the guide sequence region has m nucleotides, and any one of the mutated nucleotides is located at the position of the n^{th} nucleotide starting from the second end, 1≤n≤m, where m and n are positive integers: preferably 1≤n≤m/2, more preferably 1≤n≤m/3.

In some embodiments, in the method according to the present disclosure, m is any integer from 15 to 30, preferably any integer from 15 to 25;
optionally, any one of the mutated nucleotides is located at a position of the 1^{st} to 10^{th} nucleotides, preferably at a position of the 2^{nd} to 10^{th} nucleotides, more preferably at a position of the 2^{nd} to 7^{th} nucleotides, more preferably at a position of the 2^{nd} to 6^{th} nucleotides, starting from the second end.

In some embodiments, in the method according to the present disclosure, the mutated nucleotide contains the substitution, deletion or insertion of 1 to 10 bases, preferably the substitution, deletion or insertion of 1 to 5 bases, more preferably the substitution, deletion or insertion of 1 to 3 bases.

In the second aspect, the present disclosure provides a method for narrowing an editing window of a base editor, wherein the method comprises constructing a gRNA mutant by the method according to the first aspect: preferably, the editing window of the base editor is one base.

In the third aspect, the present disclosure provides a gRNA mutant, wherein the gRNA mutant is constructed by the method according to the first aspect: preferably, the gRNA mutant is used for a base editor with an editing window being a single nucleotide site:
preferably, the gRNA mutant comprises a structure shown in either 5′-guide sequence region-repetitive sequence region-3′ or 5′-repetitive sequence region-guide sequence region-3′.

In some embodiments, the guide sequence region in the gRNA mutant according to the present disclosure has a first end proximal to a PAM sequence of the nucleic acid of interest and a second end distal to the PAM sequence; and
the guide sequence region has m nucleotides, and any one of the mutated nucleotides is located at the position of the n^{th} nucleotide starting from the second end, 1≤n≤m, where m and n are positive integers: preferably 1≤n≤m/2, more preferably 1≤n≤m/3.

In some embodiments, the gRNA mutant according to the present disclosure, wherein m is any integer from 15 to 30, preferably any integer from 15 to 25:
optionally, any one of the mutated nucleotides is located at a position of the 1^{st} to 12^{th} nucleotides, preferably at a position of the 2^{nd} to 10^{th} nucleotides, more preferably at a position of the 2^{nd} to 7^{th} nucleotides, more preferably at a position of the 2^{nd} to 6^{th} nucleotides, starting from the second end.

In some embodiments, the mutated nucleotide in the gRNA mutant according to the present disclosure contains a substitution, deletion or insertion of 1 to 10 bases, preferably a substitution, deletion or insertion of 1 to 5 bases, more preferably a substitution, deletion or insertion of 1 to 3 bases.

In the fourth aspect, the present disclosure provides an isolated polynucleotide, wherein the isolated polynucleotide encodes the gRNA mutant according to the third aspect.

In the fifth aspect, the present disclosure provides a recombinant expression vector, wherein the recombinant expression vector contains the isolated polynucleotide according to claim 11.

In the sixth aspect, the present disclosure provides a recombinant host cell, wherein the recombinant host cell contains the recombinant expression vector according to claim 12.

In the seventh aspect, the present disclosure provides a base editor, wherein the base editor comprises either of the following (i) and (ii) and either of the following (iii) and (iv):
(i) the gRNA mutant according to the third aspect;
(ii) a polynucleotide, recombinant expression vector or recombinant host cell that expresses the gRNA mutant according to (i);
(iii) a fusion protein, wherein the fusion protein contains a first domain binding to the gRNA and a second domain having a base modification activity; and
(iv) a polynucleotide, recombinant expression vector or recombinant host cell that expresses the fusion protein as shown in (iii);
preferably, the first domain is a Cas protein mutant, homologue or polypeptide fragment having a lost or reduced nuclease activity;
optionally, the first domain is at least one selected from the group consisting of: a Cas9 protein mutant, homologue or polypeptide fragment having a lost or reduced nuclease activity and a Cas12a protein mutant, homologue or polypeptide fragment having a lost or reduced nuclease activity: preferably, the first domain is SpdCas9, SpnCas9, SadCas9, SanCas9, or LbdCpf1.

In some embodiments, the second domain in the base editor according to the present disclosure is a polypeptide having a deaminase activity: optionally, the second domain is an adenine deaminase or a mutant, homologue or polypeptide fragment having or partially having an adenine deaminase activity of the adenine deaminase: optionally, the second domain is a cytosine deaminase or a mutant, homologue or polypeptide fragment having or partially having a cytosine deaminase activity of the cytosine deaminase:
optionally, the second domain is an enzyme having the adenine deaminase activity, wherein the enzyme having the adenine deaminase activity is at least one selected from the group consisting of the following (c₁) and (c₂):
(c₁) an Escherichia coli-derived adenosine deaminase, a human-derived adenosine deaminase, or a mouse-derived adenosine deaminase; and
(c₂) a mutant, homologue or polypeptide of the adenosine deaminase as shown in (c₁) that has or partially has an adenosine deaminase activity;
optionally, the second domain is an enzyme having the cytosine deaminase activity, wherein the enzyme having the cytosine deaminase activity is at least one selected from the group consisting of the following (d₁) and (d₂):
(d₁) AID, APOBEC3A, APOBEC3G, APOBEC1, or CDA1; and
(d₂) a mutant, homologue or polypeptide of an enzyme as shown in (d₁) that has or partially has the cytosine deaminase activity.

In the eighth aspect, the present disclosure provides a composition, wherein the composition comprises the gRNA mutant according to the third aspect, the isolated polynucleotide according to the fourth aspect, the recombinant expression vector according to the fifth aspect, the recombinant host cell according to the sixth aspect, or the base editor according to the seventh aspect:
optionally, the composition further comprises one or more pharmaceutically acceptable carriers.

In the ninth aspect, the present disclosure provide use of the gRNA mutant according to the third aspect, the isolated polynucleotide according to the fourth aspect, the recombinant expression vector according to the fifth aspect, the recombinant host cell according to the sixth aspect, the base editor according to the seventh aspect, or the composition according to the eighth aspect in at least one of the following (a) and (b):
(a) serving as or preparing a reagent or kit for single-base editing; and
(b) serving as or preparing a medication for gene therapy.

In the tenth aspect, the present disclosure provides a method for gene editing in a cell or subject, wherein the method comprises bringing the cell or subject into contact with any one of the gRNA mutant according to the third aspect, the isolated polynucleotide according to the fourth aspect, the recombinant expression vector according to the fifth aspect, the recombinant host cell according to the sixth aspect, the base editor according to the seventh aspect, or the composition according to the eighth aspect:
preferably, the gene editing is editing of a single base: more preferably, the gene editing is a substitution of one base.

In the eleventh aspect, the present disclosure provides a method for treating or preventing a disease, wherein the method comprises administering to a subject the gRNA mutant according to the third aspect, the isolated polynucleotide according to the fourth aspect, the recombinant expression vector according to the fifth aspect, the recombinant host cell according to the sixth aspect, the base editor according to the seventh aspect, or the composition according to the eighth aspect:
optionally, a route of the administration includes: intravenous administration, intraperitoneal administration, intracoronary administration, intra-arterial administration, intradermal administration, subcutaneous administration, transdermal delivery, intratracheal administration, intra-articular administration, intraventricular administration, inhalation, intracerebral administration, transumbilical administration, oral administration, intraocular administration, pulmonary administration, catheter injection, administration via a suppository, a viral vector, and a lipid nanomaterial, and direct injection into a tissue.

### Effects of Invention

In some embodiments, the method for constructing a gRNA mutant provided in the present disclosure enables an acquisition of the gRNA mutant that does not perfectly match the target sequence of the nucleic acid of interest by mutating the guide sequence region in the gRNA. The gRNA mutant, when applied in a base editor, could significantly narrow the editing window of the base editor, improve the specificity of the gene editing, and has wide application prospects in gene therapy, drug screening, construction of animal and plant models, and other aspects. Besides, the gRNA mutants obtained by the construction method of the present disclosure further have an improved base editing efficiency and an optimized base editing effect.

In some embodiments, the method for constructing a gRNA mutant provided in the present disclosure allows for construction of a gRNA mutant that enables the editing window of the base editor to be accurate to one base, which has a high base editing specificity and editing efficiency, and is particularly useful for the treatment of genetic diseases caused by single-base mutations.

In some embodiments, the method for narrowing an editing window of a base editor provided in the present disclosure enables a base editor with a remarkably narrowed base editing window to be obtained without affecting the gene editing efficiency by modifying the gRNA using the method for constructing a gRNA mutant according to the present application, which has wide application prospects in the fields such as animal model construction, researches on functional genomics, molecular breeding, clinical medicine, and translational medicine.

In some embodiments, the base editor provided in the present disclosure has a narrowed base editing window or may even allow the editing window to be narrowed to one base, which could efficiently and precisely correct a single-site mutation, providing an efficient and precise editing tool for the treatment of genetic diseases, etc.

### BRIEF DESCRIPTION OF THE DRAWINGS

FIG. 1 shows a schematic diagram for precise editing of one base by a gRNA mutant-containing base editor as constructed in the present disclosure.

FIG. 2 shows a schematic diagram for a base editor to precisely edit a base at any site by changing the PAM frame in Example 3. The Y axis represents the editing efficiency.

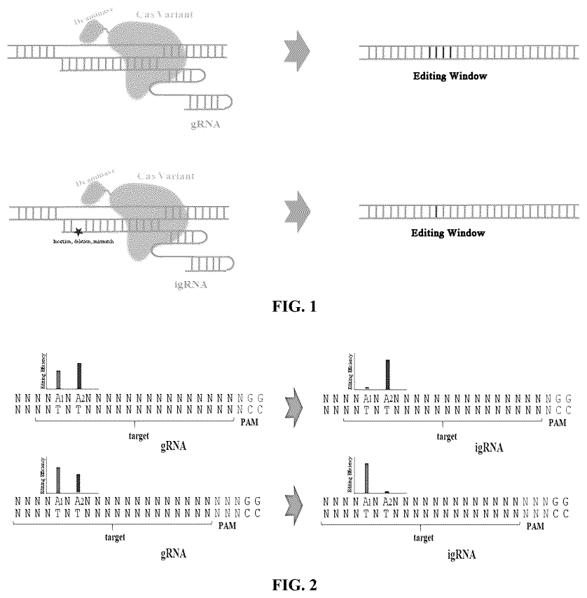

### DETAILED DESCRIPTION

When used in combination with the term “including” in the claims and/or specification, the word “a” or “an” may refer to “one”, or refer to “one or more”, “at least one”, and “one or more than one”.

As used in the claims and specification, the term “including”, “having”, “comprising”, or “containing” is intended to be inclusive or open-ended, and does not exclude additional or unrecited elements or methods and steps.

Throughout the application document, the term “about” means that a value includes the error or standard deviation caused by the device or method used to measure the value.

It is applicable to the content disclosed herein that the term “or” is defined only as alternatives and “and/or”, but the term “or” used herein refers to “and/or” unless otherwise expressly stated to be only alternatives or mutual exclusion between alternatives.

As used herein, the terms “polypeptide”, “peptide”, and “protein” are used interchangeably herein and are an amino acid polymer of any length. This polymer may be linear or branched. It may contain modified amino acids, and it may be spaced by non-amino acids. This term also includes amino acid polymers that have already been modified (e.g., formation of disulfide bonds, glycosylation, lipidation, acetylation, phosphorylation, or any other operations such as conjugation with a labeled component).

As used herein, the term “editing window” refers to the region covered by editable bases that are located within the guide sequence region of gRNA and have an editing efficiency greater than the average editing efficiency of all editable bases. Exemplarily, the adenine base editor edits the adenines (A) present in the guide sequence region, and the adenines (A) constitute editable bases within the guide sequence region. If there is i (i≥1) A within the guide sequence region, where the base editing efficiency of Aₓ (x is any integer from 1 to i) is greater than the average editing efficiency of A₁ to Aᵢ, then Aₓ constitutes the coverage region of the editing window. In some expressions of the present disclosure, “narrowing of editing window” and “improvement of single-base editing specificity” have the same meaning.

As used herein, the term “CRISPR” refers to clustered regularly interspaced short palindromic repeats, which comes from the immune system of microorganisms.

As used herein, the term “Cas protein mutant” includes Cas protein mutants, homologues or polypeptide fragments thereof that have a lost or reduced endonuclease activity as compared to a wild-type Cas protein.

As used herein, the term “wild-type Cas protein” refers to a CRISPR-associated protein. The Cas protein and the CRISPR sequence jointly form the CRISPR/Cas system. The Cas protein has a nuclease-associated functional domain, and cleaves a target sequence at a specific position by recognizing PAM (protospacer adjacent motif).

As used herein, the term “fusion protein” refers to a hybrid polypeptide comprising protein domains from at least two different proteins. The fusion protein may be a chimeric protein produced by ligating two or more genes that initially encode the separate proteins. Translation of the fusion gene produces a single polypeptide with functional characteristics derived from each of the original proteins.

As used herein, said “gRNA” is also referred to as a guide RNA, and has the meaning typically understood by a person skilled in the art. In general, the guide RNA may include a direct repeat sequence and a guide sequence, or consists essentially of or consists of a direct repeat sequence and a guide sequence (also referred to as a spacer in the context of the endogenous CRISPR system). In different CRISPR systems, the gRNA may include crRNA and tracrRNA or may contain only crRNA, according to different Cas proteins on which the gRNA depends. The crRNA and tracrRNA may be artificially modified and fused to form single guide RNA (sgRNA).

As used herein, the term “protospacer adjacent motifs (PAMs)” refers to sequences adjacent to the target sequence recognized by the Cas protein, which may be located at the 3′ end (e.g., the CRISPR/Cpf1 system) of the target sequence, or may be located at the 5′ end (e.g., the CRISPR/Cas9 system) of the target sequence.

As used herein, the term “target sequence” refers to the nucleotide sequence in the nucleic acid of interest that is complementary to or at least partially complementary to the gRNA. In the present disclosure, “target sequence” and “target nucleic acid” may be used interchangeably.

As used herein, the term “target strand” refers to the nucleotide strand in the nucleic acid of interest that hybridizes with the gRNA: the term “non-target strand” refers to the nucleotide strand in the nucleic acid of interest that does not hybridize or pair with the gRNA.

As used herein, the term “polynucleotide” refers to a polymer composed of nucleotides. A polynucleotide may be in the form of an individual fragment, or may be a constituent part of a larger nucleotide sequence structure, which is derived from the nucleotide sequence that has been isolated at least once in number or concentration, and could be recognized, operated, and sequence recovered as well as nucleotide sequence recovered by a standard molecular biological method (e.g., using a cloning vector). When a nucleotide sequence is represented by a DNA sequence (i.e., A, T, G, C), it also includes an RNA sequence (i.e., A, U, G, C), where “U” substitutes for “T”. In other words, “polynucleotide” refers to a nucleotide polymer knocked out from an additional nucleotide (an individual fragment or an entire fragment), or may be a constituent part or component of a larger nucleotide structure, such as an expression vector or a polycistronic sequence. Polynucleotide includes DNA, RNA, and cDNA sequences. “Recombinant polynucleotide” or “recombinant nucleic acid molecule” is one of “polynucleotides”.

As used herein, the term “hybridization” refers to any process of pairing complementary nucleic acids by binding a nucleic acid strand to a complementary strand through base pairing to form a hybrid complex.

As used herein, the term “mutant” refers to a polynucleotide or polypeptide that, relative to the “wild-type” or “comparative” polynucleotide or polypeptide, contains alternation(s) (i.e., substitution, insertion and/or deletion of a polynucleotide) at one or more (e.g., several) positions, where the substitution refers to a substitution of a different nucleotide for a nucleotide that occupies one position: the deletion refers to removal of a nucleotide that occupies certain position; and the insertion refers to an addition of a nucleotide after the nucleotide adjacent to and immediately following the occupied position. In the present disclosure, alternations of nucleotides also correspond to alternations of bases, and the substituted, inserted or deleted nucleotides correspond to the substituted, inserted or deleted bases.

As used herein, the term “mutated nucleotide” or “mutation of nucleotide” includes “substitution, deletion or addition of one or more nucleotides”. In the present disclosure, the term “mutation” refers to an alternation of a nucleotide sequence.

As used herein, the terms “sequence identity” and “percent identity” refer to the percentage of nucleotides or amino acids that are the same (i.e., identical) between two or more polynucleotides or polypeptides. The sequence identity between two or more polynucleotides or polypeptides may be determined by aligning the nucleotide sequences of polynucleotides or the amino acid sequences of polypeptides and scoring the number of positions at which nucleotide or amino acid residues are identical in the aligned polynucleotides or polypeptides, and comparing the number of these positions with the number of positions at which nucleotide or amino acid residues are different in the aligned polynucleotides or polypeptides. Polynucleotides may differ at one position by, e.g., containing a different nucleotide (i.e., substitution or mutation) or deleting a nucleotide (i.e., insertion or deletion of a nucleotide in one or two polynucleotides). Polypeptides may differ at one position by, e.g., containing a different amino acid (i.e., substitution or mutation) or deleting an amino acid (i.e., insertion or deletion of an amino acid in one or two polypeptides). The sequence identity may be calculated by dividing the number of positions at which nucleotide or amino acid residues are identical by the total number of nucleotide or amino acid residues in the polynucleotides or polypeptides. For example, the percent identity may be calculated by dividing the number of positions at which nucleotide or amino acid residues are identical by the total number of nucleotide or amino acid residues in the polynucleotides or polypeptides, and multiplying the result by 100.

Exemplarily, two or more sequences or subsequences, when compared and aligned at maximum correspondence by the sequence alignment algorithm or by the visual inspection measurement, have at least 80%, 81%, 82%, 83%, 84%, 85%, 86%, 87%, 88%, 89%, 90%, 91%, 92%, 93%, 94%, 95%, 96%, 97%, 98%, or 99% “sequence identity” or “percent identity.” of nucleotides. In some embodiments, the sequences are substantially identical over the full length of either or both of the compared biopolymers (e.g., polynucleotides).

As used herein, the terms “individual” and “subject” may be used interchangeably and refer to mammals. Mammals include, but are not limited to, domesticated animals (e.g., dairy cows, sheep, cats, dogs, and horses), primates (e.g., humans and non-human primates such as monkeys), rabbits, and rodents (e.g., mice and rats). In particular, the individual is a human.

As used herein, the term “vector” refers to a DNA construct containing DNA sequences operably linked to appropriate regulatory sequences, so as to express a gene of interest in a suitable host.

As used herein, the term “recombinant expression vector” refers to a DNA structure for expressing, e.g., a polynucleotide encoding the desired polypeptide. The recombinant expression vector may include, for example, transcription subunits containing i) a set of genetic elements having a regulatory effect on gene expression, such as promoters and enhancers: ii) a structure or coding sequence transcribed into mRNA and translated into a protein; and iii) an appropriate transcriptional and translational initiation and termination sequences. The recombinant expression vector is constructed in any suitable manner. The nature of the vector is not important, and any vector may be used, including plasmids, viruses, phages, and transposons. Possible vectors for use in the present disclosure include, but are not limited to, chromosomes, non-chromosomes, and synthetic DNA sequences, for example, viral plasmids, bacterial plasmids, phage DNAs, yeast plasmids, and vectors derived from combinations of plasmids and phage DNAs, and DNAs from viruses such as lentivirus, adeno-associated viruses, retroviruses, cowpox, adenoviruses, avian pox, baculoviruses, SV40, and Pseudorabies virus.

As used herein, the term “host cell” refers to a cell in which an exogenous polynucleotide has already been introduced, including progenies of such a cell. The host cell includes “transformants” and “transformed cells”, which include cells transformed from primary cells and progenies derived therefrom. The host cell is any type of cell system that can be used to produce the antibody molecules of the present disclosure, including eukaryotic cells, such as mammalian cells, insect cells, and yeast cells; and prokaryotic cells, such as Escherichia coli cells. The host cell includes cultured cells and also includes cells in transgenic animals, transgenic plants, or in cultured plant tissues or animal tissues.

As used herein, “treatment” means that after afflicting from a disease, a subject is brought into contact (e.g., administered) with the cyclic RNA, linear RNA, recombinant nucleic acid molecule, recombinant expression vector, or composition of the present disclosure, thereby alleviating symptoms of the disease as compared to the case of no contact, which does not mean that the symptoms of the disease have to be completely suppressed. Affliction from a disease refers to occurrence of symptoms of the disease in the body.

As used herein, “prevention” means that before afflicting from a disease, a subject is brought into contact (e.g., administered) with the cyclic RNA, linear RNA, recombinant nucleic acid molecule, recombinant expression vector, composition or the like of the present disclosure, thereby alleviating symptoms after affliction from the disease as compared to the case of no contact, which does not mean that illness has to be completely suppressed.

Unless otherwise defined or expressly indicated by the context, all technical and scientific terms used herein have the same meanings as typically understood by one of ordinary skill in the art to which the present disclosure belongs.

Construction Method for gRNA Mutant

The present disclosure provides a method for constructing a gRNA mutant, comprising the following steps:
a mutation step: mutating a guide sequence region in a gRNA that is hybridized with a target sequence of a nucleic acid of interest, such that a substitution, deletion or insertion of one or more bases occurs at one or more positions of the guide sequence region, to form a mutation sequence region containing a mutated nucleotide; and
a screening step: screening a mutant with a narrowed editing window for a base editor as compared to an unmutated gRNA, to obtain the gRNA mutant.

The method for constructing a gRNA mutant according to the present disclosure enables an acquisition of the gRNA mutant that is incompletely complementary to the target sequence of the nucleic acid of interest by mutating the guide sequence region in the gRNA, and the gRNA mutant is also referred to as an imperfect guide-RNA (igRNA). Compared with the gRNA, the igRNA has a narrowed editing window for the base editor, and could effectively improve the base editing specificity, and improves the base editing efficiency to some extent, resolving the defect that gene editing with high specificity at a specific site is difficult to realize because there are many base editing sites within the editing window of an available base editor.

In some preferred embodiments, the screening step comprises: screening the mutant with an editing window for the base editor being a single base, to obtain the gRNA mutant. The gRNA mutant, after applied to the base editor, is capable of realizing the precise modification of a specific single base, which provides a base editing tool with high specificity for gene therapy of diseases, animal model construction, molecular breeding, etc., and is particularly suitable for correction of pathogenic single-base mutations in genetic diseases.

In some embodiments, the guide sequence region has a first end proximal to a PAM sequence of the nucleic acid of interest and a second end distal to the PAM sequence: the guide sequence region has m nucleotides, and any one of the mutated nucleotides is located at the position of the n^{th} nucleotide starting from the second end, 1≤n≤m, where m and n are positive integers.

The present disclosure has discovered that the position of the base mutation in gRNA has a great influence on the size of the editing window; which is specifically subject to the number of nucleotides spaced between the mutated nucleotide and the PAM sequence in the nucleic acid of interest.

In some preferred embodiments, 2≤n≤m/2. In some more preferred embodiments, 2≤n≤m/3. By introducing mutant nucleotides at the aforementioned positions, the editing windows of the gRNA mutants applied to the base editor can be effectively narrowed, providing unequivocal and implementable modification positions for gRNA modifications.

In the present disclosure, the number of nucleotides in the guide sequence region is not specifically limited, and its concrete number may be designed according to the type of the Cas protein for the base editor and the target sequence bound.

In some alternative embodiments, the number m of nucleotides in the guide sequence region is any integer from 15 to 30. In some preferred embodiments, the number m of nucleotides in the guide sequence region is any integer from 15 to 25. In some preferred embodiments, the gRNA mutant is applied to the CRISPR/Cas9 system, and the number m of nucleotides in the guide sequence region is any integer from 19 to 21. Furthermore, any one of the mutated nucleotides is located at a position of the 1^{st} to 12^{th} nucleotides, preferably at a position of the 2^{nd} to 10^{th} nucleotides, more preferably at a position of the 2^{nd} to 6^{th} nucleotides, more preferably at a position of the 2^{nd} to 6^{th} nucleotides, starting from the second end.

Exemplarily, the gRNA mutant is applied to the CRISPR/Cas9 system, and the guide sequence region has 20 nucleotides. Accordingly, any one of the mutated nucleotides is located at a position of the 1^{st} to 10^{th} nucleotides, preferably at a position of the 2^{nd} to 10^{th} nucleotides, more preferably at a position of the 2^{nd} to 6^{th} nucleotides, starting from the second end. Exemplarily, any one of the mutated nucleotides is located at the position of the 1^{st}, 2^{nd}, 3^{rd}, 4^{th} 5^{th}, 6^{th}, 7^{th}, 8^{th}, 9^{th}, or 10^{th} nucleotide starting from the second end.

Exemplarily, the gRNA mutant is applied to the CRISPR/Cas9 system, and the guide sequence region has 21 nucleotides. Accordingly, any one of the mutated nucleotides is located at a position of the 1^{st} to 10^{th} nucleotides, preferably at a position of the 2^{nd} to 10^{th} nucleotides, more preferably at a position of the 2^{nd} to 6^{th} nucleotides, starting from the second end. Exemplarily, any one of the mutated nucleotides is located at the position of the 1^{st}, 2^{nd}, 3^{rd}, 4^{th}, 5^{th}, 6^{th}, 7^{th}, 8^{th}, 9^{th}, or 10^{th} nucleotide starting from the second end.

Exemplarily, the gRNA mutant is applied to the CRISPR/Cas9 system, and the guide sequence region has 22 nucleotides. Accordingly, any one of the mutated nucleotides is located at a position of the 1^{st} to 11^{th} nucleotides, preferably at a position of the 2^{nd} to 11^{th} nucleotides, more preferably at a position of the 2^{nd} to 6^{th} nucleotides, starting from the second end. Exemplarily, any one of the mutated nucleotides is located at the position of the 1^{st}, 2^{nd}, 3^{rd}, 4^{th} 5^{th}, 6^{th}, 7^{th}, 8^{th}, 9^{th}, 10^{th}, or 11^{th} nucleotide starting from the second end.

Exemplarily, the gRNA mutant is applied to the CRISPR/Cas9 system, and the guide sequence region has 23 nucleotides. Accordingly, any one of the mutated nucleotides is located at a position of the 1^{st} to 11^{th} nucleotides, preferably at a position of the 2^{nd} to 11^{th} nucleotides, more preferably at a position of the 2^{nd} to 6^{th} nucleotides, starting from the second end.

Exemplarily, any one of the mutated nucleotides is located at the position of the 1^{st}, 2^{nd}, 3^{rd}, 4^{th} 5^{th}. 6^{th}, 7^{th}, 8^{th}, 9^{th}, 10^{th}, or 11^{th} nucleotide starting from the second end.

Exemplarily, the gRNA mutant is applied to the CRISPR/Cas9 system, and the guide sequence region has 24 nucleotides. Accordingly, any one of the mutated nucleotides is located at a position of the 1^{st} to 12^{th} nucleotides, preferably at a position of the 2^{nd} to 12^{th} nucleotides, more preferably at a position of the 2^{nd} to 7^{th} nucleotides, starting from the second end. Exemplarily, any one of the mutated nucleotides is located at the position of the 1^{st}, 2^{nd}, 3^{rd}, 4^{th} 5^{th}, 6^{th}, 7^{th}, 8^{th}, 9^{th}, 10^{th}, 11^{th}, or 12^{th} nucleotide starting from the second end.

Exemplarily, the gRNA mutant is applied to the CRISPR/Cas9 system, and the guide sequence region has 25 nucleotides. Accordingly, any one of the mutated nucleotides is located at a position of the 1^{st} to 12^{th} nucleotides, preferably at a position of the 2^{nd} to 12^{th} nucleotides, more preferably at a position of the 2^{nd} to 7^{th} nucleotides, starting from the second end. Exemplarily, any one of the mutated nucleotides is located at the position of the 1^{st}, 2^{nd}, 3^{rd}, 4^{th} 5^{th}, 6^{th}, 7^{th}, 8^{th}, 9^{th}, 10^{th}, 11^{th}, or 12^{th} nucleotide starting from the second end.

Exemplarily, the gRNA mutant is applied to the CRISPR/Cas9 system, and the guide sequence region has 19 nucleotides. Accordingly, any one of the mutated nucleotides is located at a position of the 1^{st} to 9^{th} nucleotides, preferably at a position of the 2^{nd} to 9^{th} nucleotides, more preferably at a position of the 2^{nd} to 5^{th} nucleotides, starting from the second end. Exemplarily, any one of the mutated nucleotides is located at the position of the 1^{st}, 2^{nd}, 3^{rd}, 4^{th} 5^{th}, 6^{th}, 7^{th}, 8^{th}, or 9^{th} nucleotide starting from the second end.

Exemplarily, the gRNA mutant is applied to the CRISPR/Cas9 system, and the guide sequence region has 18 nucleotides. Accordingly, any one of the mutated nucleotides is located at a position of the 1^{st} to 9^{th} nucleotides, preferably at a position of the 2^{nd} to 9^{th} nucleotides, more preferably at a position of the 2^{nd} to 5^{th} nucleotides, starting from the second end. Exemplarily, any one of the mutated nucleotides is located at the position of the 1^{st}, 2^{nd}, 3^{rd}, 4^{th} 5^{th}, 6^{th}, 7^{th}, 8^{th}, or 9^{th} nucleotide starting from the second end.

Exemplarily, the gRNA mutant is applied to the CRISPR/Cas9 system, and the guide sequence region has 17 nucleotides. Accordingly, any one of the mutated nucleotides is located at a position of the 1^{st} to 8^{th} nucleotides, preferably at a position of the 2^{nd} to 8^{th} nucleotides, more preferably at a position of the 2^{nd} to 5^{th} nucleotides, starting from the second end. Exemplarily, any one of the mutated nucleotides is located at the position of the 1^{st}, 2^{nd}, 3^{rd}. 4^{th} 5^{th}, 6^{th}, 7^{th}, or 8^{th} nucleotide starting from the second end.

Exemplarily, the gRNA mutant is applied to the CRISPR/Cas9 system, and the guide sequence region has 16 nucleotides. Accordingly, any one of the corresponding mutated nucleotides is located at a position of the 1^{st} to 8^{th} nucleotides, preferably at a position of the 2^{nd} to 8^{th} nucleotides, more preferably at a position of the 2^{nd} to 5^{th} nucleotides, starting from the second end. Exemplarily, any one of the mutated nucleotides is located at the position of the 1^{st}, 2^{nd}, 3^{rd}, 4^{th}, 5^{th}, 6^{th}, 7^{th}, or 8^{th} nucleotide starting from the second end.

Exemplarily, the gRNA mutant is applied to the CRISPR/Cas9 system, and the guide sequence region has 15 nucleotides. Accordingly, any one of the mutated nucleotides is located at a position of the 1^{st} to 7^{th} nucleotides, preferably at a position of the 2^{nd} to 7^{th} nucleotides, more preferably at a position of the 2^{nd} to 4^{th} nucleotides, starting from the second end. Exemplarily, any one of the mutated nucleotides is located at the position of the 1^{st}, 2^{nd}, 3^{rd}, 4^{th} 5^{th}, 6^{th}, or 7^{th} nucleotide starting from the second end.

In other embodiments, m and n may also be other values, which are not exhausted in the present disclosure.

In some embodiments, the mutated nucleotide comprises a substitution, deletion or insertion of 1 to 10 bases, preferably a substitution, deletion or insertion of 1 to 5 bases, more preferably a substitution, deletion or insertion of 1 to 3 bases. Exemplarily, the mutated nucleotide comprises substitutions, deletions, or insertions of 1, 2, 3, 4, 5, 6, 7, 8, 9, and 10 bases. The present disclosure has found that the number of bases included in the mutated nucleotide directly influences the specificity and efficiency of the gRNA mutant for base editing. When the number of the mutated bases is within the above range, it is possible to obtain a gRNA mutant with high specificity and with an editing window optimally narrowed to one base.

It is to be noted that the mutated bases may be located at any one or more positions of the 1^{st} nucleotide to the n^{th} nucleotide starting from the second end in the guide sequence region. The mode of mutation in a base at any position may be any one independently selected from the substitution, deletion, and insertion. As long as the total number of mutated bases included in the mutated nucleotides falls within the range provided in the present disclosure, the specificity of the gRNA for gene editing may be optimized.

Furthermore, the number of mutated bases included in the mutated nucleotides is further determined by the type of the base editor.

In some preferred embodiments, the base editors are adenine base editors (ABEs). The mutated nucleotides of the gRNA mutant for the adenine base editor contain a substitution, deletion or insertion of 1 to 5 bases, preferably a substitution, deletion or insertion of 1 to 3 bases, more preferably a substitution, deletion or insertion of one base. Exemplarily, the mutated nucleotides of the gRNA mutant for the adenine base editor contain a substitution, deletion or insertion of 1 base, 2 bases, 3 bases, 4 bases, 5 bases and so forth. The present disclosure has found in the experiments that when the number of mutated bases in the gRNA mutants of some genes is set as 1, an adenine base editor capable of mutating adenine at one specific site is obtained.

In some preferred embodiments, the base editors are cytosine base editors (CBEs). The mutated nucleotides of the gRNA mutant for the cytosine base editor contain a substitution, deletion or insertion of 1 to 5 bases, preferably a substitution, deletion or insertion of 1 to 3 bases, more preferably substitutions, deletions or insertions of 2 or 3 bases. Exemplarily, the mutated nucleotides of the gRNA mutant for the cytosine base editor contain a substitution, deletion or insertion of 1 base, 2 bases, 3 bases, 4 bases, 5 bases and so forth. The present disclosure has found in the experiments that when the number of mutated bases in the gRNA mutants of some genes is set as 2 or 3, a cytosine base editor capable of editing cytosine at one specific site is obtained.

gRNA Mutant

The present disclosure provides a gRNA mutant, which is obtained by the construction method according to the present disclosure. The gRNA mutant, when used for the base editor, is capable of effectively improving the specificity of the base editing, narrowing the editing window for the base editor, and finally realizing efficient and specific editing of a particular single base, thereby providing a positive and efficient gene editing strategy for treatment of diseases and construction of animal and plant models.

Furthermore, the gRNA mutant further comprises a repetitive sequence region for binding a Cas protein. The repetitive sequence region may be folded to form a specific structure (such as a stem-loop structure) to be recognized by the Cas protein. After binding to the gRNA mutant, the Cas protein recognizes the target sequence by recognizing the PAM (protospacer adjacent motif) sequence adjacent to the target sequence of the nucleic acid of interest. After the mutation sequence region of the gRNA mutant is hybridized with the target sequence, the double strands of the nucleic acid of interest are unwinded to form an R-loop region, attaining a ternary complex of the Cas protein-gRNA-target sequence, and the single-stranded nucleic acid strand of the R-loop region is further edited. Depending upon the difference in CRISPR/Cas systems to which the gRNA mutants are applied, the repetitive sequence region may be linked to the 5′ end or the 3′ end of the guide sequence region. In some alternative embodiments, the gRNA mutant is applied to the CRISPR/Cas9 system, and the gRNA mutant includes the nucleotide sequence of 5′-guide sequence region-repetitive sequence region-3′. In some alternative embodiments, the gRNA mutant is applied to the CRISPR/Cpf1 system, and the gRNA mutant includes the nucleotide sequence of 5′-repetitive sequence region-guide sequence region-3′.

In the present disclosure, the nucleotide sequence of the mutation sequence region of the gRNA mutant is not specifically limited, so long as the sequence of the mutation region thereof enables the gRNA mutant to be hybridized with the target sequence to form a double strand and to unwind the nucleic acid of interest to form a R-loop region; and enables the editing window of the gRNA mutant for the base editor to be narrowed.

In some alternative embodiments, the gRNA mutant is used to mutate the base C at a specific site of the HIRA locus into base T, and the nucleotide sequence of the guide sequence region of the unmutated gRNA is set forth in SEQ ID NO: 1. The nucleotide sequence of the mutation sequence region of the gRNA mutant (hereinafter referred to as igRNA) is set forth in any one of SEQ ID NOs: 2 to 5. Of these, the sequence set forth in SEQ ID NO: 2 has a substitution of one base at the position corresponding to the 3^{rd} nucleotide of the sequence set forth in SEQ ID NO: 1, the sequence set forth in SEQ ID NO: 3 has a substitution of one base at the position corresponding to the 5^{th} nucleotide of the sequence set forth in SEQ ID NO: 1, the sequence set forth in SEQ ID NO: 4 has a deletion of one base at the position corresponding to the 3^{rd} nucleotide of the sequence set forth in SEQ ID NO: 1, and the sequence set forth in SEQ ID NO: 5 has an insertion of two bases at the position corresponding to the 3^{rd} nucleotide of the sequence set forth in SEQ ID NO: 1. Compared to the gRNA whose guide sequence region is the sequence set forth in SEQ ID NO: 1, igRNAs with different modes of mutation, when applied to the cytosine base editors for editing the HIRA locus, have a universally improved base editing specificity to the C6 site, and the editing efficiency is also improved.

In some alternative embodiments, the gRNA mutant is used to mutate the base C at a specific site of the DNMT3B locus into base T, and the nucleotide sequence of the guide sequence region of the unmutated gRNA is set forth in SEQ ID NO: 6. The nucleotide sequence of the mutation sequence region of the gRNA mutant is set forth in any one of SEQ ID NOs: 7 to 10. Of these, the sequence set forth in SEQ ID NO: 7 has a substitution of one base at the position corresponding to the 3^{rd} nucleotide of the sequence set forth in SEQ ID NO: 6, the sequence set forth in SEQ ID NO: 8 has substitutions of two bases at the positions corresponding to the 3^{rd} and 4^{th} nucleotides of the sequence set forth in SEQ ID NO: 6, the sequence set forth in SEQ ID NO: 9 has a deletion of one base at the position corresponding to the 2^{nd} nucleotide of the sequence set forth in SEQ ID NO: 6, and the sequence set forth in SEQ ID NO: 10 has an insertion of two bases at the position corresponding to the 3^{rd} nucleotide of the sequence set forth in SEQ ID NO: 6. Compared to the gRNA whose guide sequence region is the sequence set forth in SEQ ID NO: 6, igRNAs with different modes of mutation, when applied to the cytosine base editors for editing the DNMT3B locus, have a universally improved base editing specificity to the C8 site, and the editing efficiency is also improved.

In some alternative embodiments, the gRNA mutant is used to mutate the base C at a specific site of the RNF2 locus into base T, and the nucleotide sequence of the guide sequence region of the unmutated gRNA is set forth in SEQ ID NO: 11. The nucleotide sequence of the mutation sequence region of the gRNA mutant is set forth in any one of SEQ ID NOs: 12 to 14. Of these, the sequence set forth in SEQ ID NO: 12 has a substitution of one base at the position corresponding to the 3^{rd} nucleotide of the sequence set forth in SEQ ID NO: 11, the sequence set forth in SEQ ID NO: 13 has a deletion of one base at the position corresponding to the 2^{nd} nucleotide of the sequence set forth in SEQ ID NO: 11, and the sequence set forth in SEQ ID NO: 14 has an insertion of one base at the position corresponding to the 3^{rd} nucleotide of the sequence set forth in SEQ ID NO: 11. Compared to the gRNA whose guide sequence region is the sequence set forth in SEQ ID NO: 11, igRNAs with different modes of mutation, when applied to the cytosine base editors for editing the RNF2 locus, have a universally improved base editing specificity to the C6 site, and the editing efficiency is also improved.

In some alternative embodiments, the gRNA mutant is used to mutate the base C at a specific site of the NSD1 locus into base T, and the nucleotide sequence of the guide sequence region of the unmutated gRNA is set forth in SEQ ID NO: 19. The nucleotide sequence of the mutation sequence region of the gRNA mutant is set forth in any one of SEQ ID NOs: 20 to 22. Of these, the sequence set forth in SEQ ID NO: 20 has a substitution of one base at the position corresponding to the 3^{rd} nucleotide of the sequence set forth in SEQ ID NO: 19, the sequence set forth in SEQ ID NO: 21 has deletions of two bases at the positions corresponding to the 2^{nd} and 3^{rd} nucleotides of the sequence set forth in SEQ ID NO: 19, and the sequence set forth in SEQ ID NO: 22 has an insertion of one base at the position corresponding to the 2^{nd} nucleotide of the sequence set forth in SEQ ID NO: 19. Compared to the gRNA whose guide sequence region is the sequence set forth in SEQ ID NO: 19, igRNAs with different modes of mutation, when applied to the cytosine base editors for editing the NSD1 locus, have a universally improved base editing specificity to the C6 site, and the editing efficiency is also improved.

In some alternative embodiments, the gRNA mutant is used to mutate the base A at a specific site of the PSMB2 locus into base G, and the nucleotide sequence of the guide sequence region of the unmutated gRNA is set forth in SEQ ID NO: 23. The nucleotide sequence of the mutation sequence region of the gRNA mutant is set forth in any one of SEQ ID NOs: 24 to 27. Of these, the sequence set forth in SEQ ID NO: 24 has a substitution of one base at the position corresponding to the 3^{rd} nucleotide of the sequence set forth in SEQ ID NO: 23, the sequence set forth in SEQ ID NO: 25 has a substitution of one base at the position corresponding to the 5^{th} nucleotide of the sequence set forth in SEQ ID NO: 23, the sequence set forth in SEQ ID NO: 26 has a deletion of one base at the position corresponding to the 3^{rd} nucleotide of the sequence set forth in SEQ ID NO: 23, and the sequence set forth in SEQ ID NO: 27 has an insertion of one base at the position corresponding to the 3^{rd} nucleotide of the sequence set forth in SEQ ID NO: 23. Compared to the gRNA whose guide sequence region is the sequence set forth in SEQ ID NO: 23, igRNAs with different modes of mutation, when applied to the cytosine base editors for editing the PSMB2 locus, have a universally improved base editing specificity to the A5 site, and the editing efficiency is also improved.

In some alternative embodiments, the gRNA mutant is used to mutate the base A at a specific site of the ABCA3 locus into base G, and the nucleotide sequence of the guide sequence region of the unmutated gRNA is set forth in SEQ ID NO: 28. The nucleotide sequence of the mutation sequence region of the gRNA mutant is set forth in any one of SEQ ID NOs: 29 to 31. Of these, the sequence set forth in SEQ ID NO: 29 has a substitution of one base at the position corresponding to the 3^{rd} nucleotide of the sequence set forth in SEQ ID NO: 28, the sequence set forth in SEQ ID NO: 30 has a deletion of one base at the position corresponding to the 3^{rd} nucleotide of the sequence set forth in SEQ ID NO: 28, and the sequence set forth in SEQ ID NO: 31 has an insertion of one base at the position corresponding to the 2^{nd} nucleotide of the sequence set forth in SEQ ID NO: 28. Compared to the gRNA whose guide sequence region is the sequence set forth in SEQ ID NO: 28, igRNAs with different modes of mutation, when applied to the cytosine base editors for editing the ABCA3 locus, have a universally improved base editing specificity to the A₅ site, and the editing efficiency is also improved.

In some alternative embodiments, the gRNA mutant is used to mutate the base A at a specific site of the EMX1-SITE3 locus into base G, and the nucleotide sequence of the guide sequence region of the unmutated gRNA is set forth in SEQ ID NO: 32. The nucleotide sequence of the mutation sequence region of the gRNA mutant is set forth in any one of SEQ ID NOs: 33 to 36. Of these, the sequence set forth in SEQ ID NO: 33 has a substitution of one base at the position corresponding to the 3^{rd} nucleotide of the sequence set forth in SEQ ID NO: 32, the sequence set forth in SEQ ID NO: 34 has a substitution of one base at the position corresponding to the 4^{th} nucleotide of the sequence set forth in SEQ ID NO: 32, the sequence set forth in SEQ ID NO: 35 has a deletion of one base at the position corresponding to the 2^{nd} nucleotide of the sequence set forth in SEQ ID NO: 32, and the sequence set forth in SEQ ID NO: 36 has an insertion of one base at the position corresponding to the 3^{rd} nucleotide of the sequence set forth in SEQ ID NO: 32. Compared to the gRNA whose guide sequence region is the sequence set forth in SEQ ID NO: 32, igRNAs with different modes of mutation, when applied to the cytosine base editors for editing the EMX1-SITE3 locus, have a universally improved base editing specificity to the A6 site, and the editing efficiency is also improved.

In some alternative embodiments, the gRNA mutant is used to mutate the base A at a specific site of the VISTA HS267 locus into base G, and the nucleotide sequence of the guide sequence region of the unmutated gRNA is set forth in SEQ ID NO: 37. The nucleotide sequence of the mutation sequence region of the gRNA mutant is set forth in any one of SEQ ID NOs: 38 to 41. Of these, the sequence set forth in SEQ ID NO: 38 has a substitution of one base at the position corresponding to the 2^{nd} nucleotide of the sequence set forth in SEQ ID NO: 37, the sequence set forth in SEQ ID NO: 39 has a substitution of one base at the position corresponding to the 4^{th} nucleotide of the sequence set forth in SEQ ID NO: 37, the sequence set forth in SEQ ID NO: 40 has a deletion of one base at the position corresponding to the 4^{th} nucleotide of the sequence set forth in SEQ ID NO: 37, and the sequence set forth in SEQ ID NO: 41 has an insertion of one base at the position corresponding to the 3^{rd} nucleotide of the sequence set forth in SEQ ID NO: 37. Compared to the gRNA whose guide sequence region is the sequence set forth in SEQ ID NO: 37, igRNAs with different modes of mutation, when applied to the cytosine base editors for editing the VISTA HS267 locus, have a universally improved base editing specificity to the A5 site, and the editing efficiency is also improved.

In some alternative embodiments, the gRNA mutant is used to mutate the base A at a specific site of the SNCA locus into base G, and the nucleotide sequence of the guide sequence region of the unmutated gRNA is set forth in SEQ ID NO: 42. The nucleotide sequence of the mutation sequence region of the gRNA mutant is set forth in any one of SEQ ID NOs: 43 to 46. Of these, the sequence set forth in SEQ ID NO: 43 has a substitution of one base at the position corresponding to the 3^{rd} nucleotide of the sequence set forth in SEQ ID NO: 42, the sequence set forth in SEQ ID NO: 44 has a substitution of one base at the position corresponding to the 5^{th} nucleotide of the sequence set forth in SEQ ID NO: 42, the sequence set forth in SEQ ID NO: 45 has a deletion of one base at the position corresponding to the 3^{rd} nucleotide of the sequence set forth in SEQ ID NO: 42, and the sequence set forth in SEQ ID NO: 46 has an insertion of one base at the position corresponding to the 3^{rd} nucleotide of the sequence set forth in SEQ ID NO: 42. Compared to the gRNA whose guide sequence region is the sequence set forth in SEQ ID NO: 42, igRNAs with different modes of mutation, when applied to the cytosine base editors for editing the SNCA locus, have a universally improved base editing specificity to the A5 site, and the editing efficiency is also improved.

In some alternative embodiments, the gRNA mutant is used to mutate the base A at a specific site of the ANO5 locus into base G, and the nucleotide sequence of the guide sequence region of the unmutated gRNA is set forth in SEQ ID NO: 47. The nucleotide sequence of the mutation sequence region of the gRNA mutant is set forth in any one of SEQ ID NOs: 48 to 51. Of these, the sequence set forth in SEQ ID NO: 48 has a substitution of one base at the position corresponding to the 2^{nd} nucleotide of the sequence set forth in SEQ ID NO: 47, the sequence set forth in SEQ ID NO: 49 has a substitution of one base at the position corresponding to the 4^{th} nucleotide of the sequence set forth in SEQ ID NO: 47, the sequence set forth in SEQ ID NO: 50 has a deletion of one base at the position corresponding to the 2^{nd} nucleotide of the sequence set forth in SEQ ID NO: 47, and the sequence set forth in SEQ ID NO: 51 has an insertion of one base at the position corresponding to the 3^{rd} nucleotide of the sequence set forth in SEQ ID NO: 47. Compared to the gRNA whose guide sequence region is the sequence set forth in SEQ ID NO: 47, igRNAs with different modes of mutation, when applied to the cytosine base editors for editing the ANO5 locus, have a universally improved base editing specificity to the A7 site, and the editing efficiency is also improved.

In some alternative embodiments, the gRNA mutant is used to mutate the base A at a specific site of the KCNQ2 locus into base G, and the nucleotide sequence of the guide sequence region of the unmutated gRNA is set forth in SEQ ID NO: 52. The nucleotide sequence of the mutation sequence region of the gRNA mutant is set forth in any one of SEQ ID NOs: 53 to 55. Of these, the sequence set forth in SEQ ID NO: 53 has a substitution of one base at the position corresponding to the 3^{rd} nucleotide of the sequence set forth in SEQ ID NO: 52, the sequence set forth in SEQ ID NO: 54 has a deletion of one base at the position corresponding to the 3^{rd} nucleotide of the sequence set forth in SEQ ID NO: 52, and the sequence set forth in SEQ ID NO: 55 has an insertion of one base at the position corresponding to the 2^{nd} nucleotide of the sequence set forth in SEQ ID NO: 52. Compared to the gRNA whose guide sequence region is the sequence set forth in SEQ ID NO: 52, igRNAs with different modes of mutation, when applied to the cytosine base editors for editing the KCNQ2 locus, have a universally improved base editing specificity to the A5 site, and the editing efficiency is also improved.

In some alternative embodiments, the gRNA mutant is used to mutate the base A at a specific site of the NOTCH2 locus into base G, and the nucleotide sequence of the guide sequence region of the unmutated gRNA is set forth in SEQ ID NO: 56. The nucleotide sequence of the mutation sequence region of the gRNA mutant is set forth in any one of SEQ ID NOs: 57 to 59. Of these, the sequence set forth in SEQ ID NO: 57 has a substitution of one base at the position corresponding to the 6^{th} nucleotide of the sequence set forth in SEQ ID NO: 56, the sequence set forth in SEQ ID NO: 58 has a deletion of one base at the position corresponding to the 4^{th} nucleotide of the sequence set forth in SEQ ID NO: 56, and the sequence set forth in SEQ ID NO: 59 has an insertion of one base at the position corresponding to the 3^{rd} nucleotide of the sequence set forth in SEQ ID NO: 56. Compared to the gRNA whose guide sequence region is the sequence set forth in SEQ ID NO: 56, igRNAs with different modes of mutation, when applied to the cytosine base editors for editing the NOTCH2 locus, have a universally improved base editing specificity to the A5 site, and the editing efficiency is also improved.

In some alternative embodiments, the gRNA mutant is used to mutate the base A at a specific site of the GFI1 locus into base G, and the nucleotide sequence of the guide sequence region of the unmutated gRNA is set forth in SEQ ID NO: 60. The nucleotide sequence of the mutation sequence region of the gRNA mutant is set forth in any one of SEQ ID NOs: 61 to 63. Of these, the sequence set forth in SEQ ID NO: 61 has a substitution of one base at the position corresponding to the 2^{nd} nucleotide of the sequence set forth in SEQ ID NO: 60, the sequence set forth in SEQ ID NO: 62 has a deletion of one base at the position corresponding to the 4^{th} nucleotide of the sequence set forth in SEQ ID NO: 60, and the sequence set forth in SEQ ID NO: 63 has an insertion of one base at the position corresponding to the 3^{rd} nucleotide of the sequence set forth in SEQ ID NO: 60. Compared to the gRNA whose guide sequence region is the sequence set forth in SEQ ID NO: 60, igRNAs with different modes of mutation, when applied to the cytosine base editors for editing the GFI1 locus, have a universally improved base editing specificity to the A5 site, and the editing efficiency is also improved.

In some alternative embodiments, the gRNA mutant is used to mutate the base A at a specific site of the CFAP61 locus into base G, and the nucleotide sequence of the guide sequence region of the unmutated gRNA is set forth in SEQ ID NO: 71. The nucleotide sequence of the mutation sequence region of the gRNA mutant is set forth in any one of SEQ ID NOs: 72 to 75. Of these, the sequence set forth in SEQ ID NO: 72 has a substitution of one base at the position corresponding to the 3^{rd} nucleotide of the sequence set forth in SEQ ID NO: 71, the sequence set forth in SEQ ID NO: 73 has substitutions of two bases at the positions corresponding to the 2^{nd} and 3^{rd} nucleotides of the sequence set forth in SEQ ID NO: 71, the sequence set forth in SEQ ID NO: 74 has a deletion of one base at the position corresponding to the 6^{th} nucleotide of the sequence set forth in SEQ ID NO: 71, and the sequence set forth in SEQ ID NO: 75 has an insertion of one base at the position corresponding to the 3^{rd} nucleotide of the sequence set forth in SEQ ID NO: 71. Compared to the gRNA whose guide sequence region is the sequence set forth in SEQ ID NO: 71, igRNAs with different modes of mutation, when applied to the cytosine base editors for editing the CFAP61 locus, have a universally improved base editing specificity to the A11 site, and the editing efficiency is also improved.

In some alternative embodiments, the gRNA mutant is used to mutate the base A at a specific site of the Query_55451 locus into base G, and the nucleotide sequence of the guide sequence region of the unmutated gRNA is set forth in SEQ ID NO: 76. The nucleotide sequence of the mutation sequence region of the gRNA mutant is set forth in any one of SEQ ID NOs: 77 to 80. Of these, the sequence set forth in SEQ ID NO: 77 has substitutions of two bases at the positions corresponding to the 2^{nd} and 3^{rd} nucleotides of the sequence set forth in SEQ ID NO: 76, the sequence set forth in SEQ ID NO: 78 has a substitution of one base at the position corresponding to the 2^{nd} nucleotide of the sequence set forth in SEQ ID NO: 76, the sequence set forth in SEQ ID NO: 79 has deletions of two bases at the positions corresponding to the 3^{rd} and 4^{th} nucleotides of the sequence set forth in SEQ ID NO: 76, and the sequence set forth in SEQ ID NO: 80 has an insertion of two bases at the position corresponding to the 3^{rd} nucleotide of the sequence set forth in SEQ ID NO: 76. Compared to the gRNA whose guide sequence region is the sequence set forth in SEQ ID NO: 76, igRNAs with different modes of mutation, when applied to the cytosine base editors for editing the Query_55451 locus, have a universally improved base editing specificity to the A9 site, and the editing efficiency is also improved.

It follows from the above that the gRNA mutants obtained by the construction method according to the present disclosure could universally improve the editing specificity of the cytosine base editor and the adenine base editor, providing a universal and effective optimization strategy for narrowing the editing window of the base editor.

In the present disclosure, the nucleotide sequence of the repetitive sequence region of the gRNA mutant is not specifically limited, so long as the sequence of the mutation region thereof enables the gRNA mutant to bind to the Cas protein and guide the Cas protein to target the target sequence that edits the nucleic acid of interest.

In some alternative embodiments, the gRNA mutant is applied to the CRISPR/spCas9 system, and the repetitive sequence region of the gRNA mutant includes a nucleotide sequence set forth in either of the following (v) and (vi):
(v) the nucleotide sequence set forth in SEQ ID NO: 81; and
(vi) a sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the nucleotide sequence set forth in SEQ ID NO: 81.

In some alternative embodiments, the gRNA mutant is applied to the CRISPR/saCas9 system, and the repetitive sequence region of the gRNA mutant includes a nucleotide sequence set forth in either of the following (vii) and (viii):
(vii) the nucleotide sequence set forth in SEQ ID NO: 82; and
(viii) a sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the nucleotide sequence set forth in SEQ ID NO: 82.

In some embodiments, the present disclosure provides an isolated polynucleotide, which encodes the gRNA mutant according to the present disclosure, and could be used for mass production of gRNA mutants.

In some embodiments, the present disclosure provides a recombinant expression vector, comprising the isolated polynucleotide according to the present disclosure. The vectors for linking polynucleotides may be of various types commonly used in the art and suitable for cell transduction in vivo or in vitro.

In some embodiments, the present disclosure provides a recombinant host cell, comprising the recombinant expression vector according to the present disclosure. The gRNA mutant according to the present disclosure could be obtained through replication and expression of the recombinant expression vector in the recombinant host cell.

### Method for Narrowing Base Editor

The method for narrowing the base editor provided in the present disclosure comprises constructing a target gRNA mutant by using the method for constructing a gRNA mutant according to the present disclosure.

The method for narrowing the base editor provided in the present disclosure could improve the base editing specificity without an influence on the base editing efficiency and effectively narrow base editing windows: moreover, the method is universal in narrowing of editing windows, and is suitable for optimizing cytosine base editors and adenine base editors to expand the applications of base editors in the fields such as animal model construction, researches on functional genomics, molecular breeding, clinical medicine, and translational medicine.

In some preferred embodiments, the method for narrowing the base editor narrows the editing window of the base editor to one base. Since most of genetic diseases are caused by single-base mutations, narrowing the window of the base editor to one base by the method according to the present disclosure could specifically correct pathogenic point mutations and avoid unnecessary base editing during the gene editing, which provides a positive and effective therapeutic tool for the treatment of genetic diseases.

### Base Editor

The base editor provided in the present disclosure comprises either of the following (i) and (ii) and either of the following (iii) and (iv):
(i) a gRNA mutant;
(ii) a polynucleotide, recombinant expression vector or recombinant host cell that expresses the gRNA mutant;
(iii) a fusion protein, wherein the fusion protein contains a first domain binding to the gRNA and a second domain having a base modification activity; and
(iv) a polynucleotide, recombinant expression vector or recombinant host cell that expresses the fusion protein as shown in (iii).

The polynucleotide that expresses the gRNA mutant may be a DNA nucleic acid molecule that is transcribed to the gRNA mutant, or an RNA nucleic acid molecule containing the gRNA mutant. The recombinant expression vector may be formed through recombination by ligating the nucleotide sequence encoding the gRNA mutant with any type of vector. For example, the recombinant expression vector is a viral vector containing the nucleotide sequence that encodes the gRNA mutant, and so forth.

The polynucleotide that expresses the fusion protein may be an RNA molecule that produces the fusion protein by translation, or a DNA molecule that produces the above RNA molecule by transcription. The recombinant expression vector may be formed through recombination by ligating the nucleotide sequence encoding the fusion protein with any type of vector. For example, the recombinant expression vector is a viral vector containing an open reading frame that encodes the fusion protein, and so forth.

In the present disclosure, the first domain and the second domain of the fusion protein may be linked directly or indirectly via a linker peptide.

The first domain is a Cas protein having a lost or reduced nuclease activity. In some embodiments, the first domain is a Cas protein mutant that loses the nuclease activity, or a homologue or polypeptide fragment thereof, and the fusion protein binds to the gRNA via the first domain and recognizes the target sequence adjacent to the PAM sequence of the nucleic acid of interest to form a ternary complex of fusion protein-gRNA mutant-target sequence. Due to loss of the nuclease activity for the Cas protein, the first domain will not cleave the target strand or non-target strand after formation of the ternary complex, and only the base modification activity of the second domain is relied on to mutate the base within the editing window of the target sequence. In some embodiments, the first domain is a Cas protein mutant with a reduced nuclease activity, or a homologue or polypeptide fragment thereof, and the first domain cleaves one of the target strands or non-target strands after formation of the ternary complex. In some embodiments, the first domain is a Cas protein mutant with an expanded PAM frame, or a homologue or polypeptide fragment thereof. Exemplarily, FIG. 2 shows an adenine base editor. If NGG is used as a PAM frame and an unmutated gRNA is used, both the base A1 and the base A2 are located within the editing window of the adenine base editor: if the gRNA mutant provided in the present disclosure is used, its editing window is narrowed to the base A2, but it is impossible to realize specific editing of the base A1. At this time, the Cas protein mutant with an expanded PAM frame is used as the first domain to move the editing window of the base editor from A2 to A1, thereby achieving the specific editing of the base A1.

In the present disclosure, there are many different choices for the Cas protein. Exemplarily, the Cas protein is selected from Cas9 proteins (WP_032462936, WP_165886160, WP 002460848, and WP_002807152), Cas12a proteins (i.e., Cpf1 proteins, PDB: 6KLB_A, PDB: 6KLB_D, and UniProtKB/Swiss-Prot: U2UMQ6), Cas12b proteins (WP_217021837, WP_163299037, and WP_027726362), Cas13a protein (UniProtKB/Swiss-Prot: PODPB8), and so forth.

In some specific embodiments, the Cas protein is Streptococcus pyogenes Cas9 (SpCas9), Staphylococcus aureus Cas9 (SaCas9), Lachnospiraceae bacterium Cpf1 (LbCpf1), etc.

In some alternative embodiments, the first domain is a SpCas9 protein mutant. Furthermore, the SpCas9 protein mutant is selected from SpCas9 protein mutants with a lost nuclease activity (SpdCas9) or SpCas9 protein mutants with a reduced nuclease activity (SpnCas9). Exemplarily, the amino acid sequence of SpdCas9 is set forth in SEQ ID NO: 85, or is a sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 85. The amino acid sequence of SpnCas9 is set forth in SEQ ID NO: 86, or is a sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 86.

In some alternative embodiments, the first domain is a SaCas9 protein mutant. Furthermore, the SaCas9 protein mutant is selected from SaCas9 protein mutants with a lost nuclease activity (SadCas9) or SaCas9 protein mutants with a reduced nuclease activity (SanCas9). Exemplarily, the amino acid sequence of SadCas9 is set forth in SEQ ID NO: 87, or is a sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 87. The amino acid sequence of SanCas9 is set forth in SEQ ID NO: 88, or is a sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 88.

In some alternative embodiments, the first domain is an LbCpf1 protein mutant. Furthermore, the LbCpf1 protein mutant is an LbCpf1 protein mutant with a lost nuclease activity (LbdCpf1). Exemplarily, the amino acid sequence of LbdCpf1 is set forth in SEQ ID NO: 89, or a sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 89.

In some embodiments, the base editor of the present disclosure enables editing of one base at any site by selecting an appropriate PAM sequence and a Cas protein recognizing the PAM sequence.

The second domain has a base modification activity. In some embodiments, the base modification activity is a deaminase activity, and the second domain is a deaminase, or a mutant, homologue or polypeptide having or at least partially having a deaminase activity. At the time of forming a ternary complex of fusion protein-gRNA mutant-target sequence, the second domain mutates the base within the editing window by deaminizing the base within the editing window.

In some embodiments, the second domain is an adenine deaminase, or a mutant, homologue or polypeptide fragment having or partially having an adenine deaminase activity. The adenine deaminase will mutate A within the editing window into G. Because of having an editing window with optimal one base, the base editor of the present disclosure could be used to realize one specific base mutation from A to G.

The amino acid sequence of the adenine deaminase, or the mutant, polypeptide or homologue thereof is not specifically limited in the present disclosure, so long as the adenine deaminase, or the mutant, polypeptide or homologue thereof could have an enzyme activity of the adenine deaminase and realize the A-to-G mutation.

In some alternative embodiments, the second domain is an enzyme having the adenine deaminase activity that is at least one selected from the group consisting of the following (c₁) and (c₂):
(c₁) an Escherichia coli-derived adenosine deaminase, a human-derived adenosine deaminase, or a mouse-derived adenosine deaminase; and
(c₂) a mutant, homologue or polypeptide of the adenosine deaminase as shown in (c₁) that has or partially has an adenosine deaminase activity.

In some alternative embodiments, the second domain is tRNA adenosine deaminase TadA from Escherichia coli, or a mutant, homologue or polypeptide in which the adenosine deaminase activity is maintained. Exemplarily, the amino acid sequence of TadA may refer to NP_417054.

In some alternative embodiments, the second domain is a mutant TadA* of TadA having an adenosine deaminase activity. Exemplarily, the amino acid sequence of TadA* is set forth in SEQ ID NO: 83 or SEQ ID NO: 84. TadA from Escherichia coli or the mutant TadA* of TadA could realize the mutation from adenine A to guanine G by deaminizing the adenine within the editing window.

In some embodiments, the second domain is a cytosine deaminase, or a mutant, homologue or polypeptide fragment having or partially having the cytosine deaminase activity. The cytosine deaminase will mutate C within the editing window into T. Because of having an editing window with optimal one base, the base editor of the present disclosure could be used to realize a specific base mutation from C to T.

The present disclosure does not specifically limit the amino acid sequence of the cytosine deaminase or the mutant, polypeptide or homologue thereof, so long as it could have an enzyme activity of the cytosine deaminase and realize the C-to-T mutation.

In some alternative embodiments, the second domain is an enzyme having the cytosine deaminase activity that is at least one selected from the group consisting of the following (d₁) and (d₂):
(d₁) AID, APOBEC3A, APOBEC3G, APOBEC1, or CDA1; and
(d₂) a mutant, homologue or polypeptide of an enzyme as shown in (d₁) that has or partially has the cytosine deaminase activity.

Exemplarily, the second domain may be any one selected from the following cytosine deaminases, or mutants, homologues or polypeptides thereof: AID (the amino acid sequence may refer to NP_001317272, NP_065712), APOBEC3A (e.g.: AKE33285, AEH96362, ACH92046, CAK54680), APOBEC3G (e.g.: human APOBEC3G, the amino acid sequence may refer to NP_068594, NP_001336365, NP_001336366, NP_001336367), APOBEC1 (e.g.: APOBEC1, the amino acid sequence may refer to NP_001127863, NP_112436; human APOBEC1, the amino acid sequence may refer to NP_001291495, NP_001635, NP_005880), CDA1 (e.g.: Lampetra japonica CDA1, ABO15149, ABO15150).

### Composition

The composition provided in the present disclosure comprises the gRNA mutant, isolated polynucleotide, recombinant expression vector, recombinant host cell or base editor according to the present disclosure. Due to the inclusion of optimized gRNAs, the base editors of the present disclosure exhibit a high specificity to the gene editing of the nucleic acid of interest, could achieve efficient editing of one base, and hold great potential in gene therapy, construction of animal and plant models, etc.

In some embodiments, the composition further comprises one or more pharmaceutically acceptable carriers. In the present disclosure, the composition is used to facilitate the administration to an organism, facilitate absorption of active ingredients, and thus exert the bioactivity. The composition of the present disclosure may be administered in any form including injection (intra-arterial, intravenous, intramuscular, intraperitoneal, or subcutaneous), mucosa administration, oral administration (an oral solid preparation or an oral liquid preparation), rectal administration, inhalation, implantation, topical administration (e.g., eyes), etc. Non-limiting examples of the oral solid preparations include, but are not limited to, powders, capsules, pastilles, granules, tablets, etc. Non-limiting examples of the liquid preparations for oral or mucosa administration include, but are not limited to, suspensions, tinctures, elixirs, solutions, etc. Non-limiting examples of the preparations for topical administration include, but are not limited to, emulsions, gels, ointments, creams, patches, pastes, foams, lotions, drops, or serum preparations. Non-limiting examples of the preparations for parenteral administration include, but are not limited to, solutions for injection, dry powders for injection, suspensions for injection, emulsions for injection, etc. The composition of the present disclosure may be further prepared into controlled-release or extended-release dosage forms (e.g., liposomes or microspheres).

In the present disclosure, the routes of administration may be varied or adapted in any applicable way to meet the requirements for the properties of drugs, the convenience of the patients and medical personnel, and other related factors.

Use of gRNA Mutant and Base Editor

In some embodiments, the present disclosure provides use of the gRNA mutant or base editor as a reagent or kit or in the preparation of a reagent or kit for single-base editing. The gRNA mutants or base editors of the present disclosure could achieve more controllable and more specific editing of one base, thereby overcoming the defect that conventional base editors cannot edit one specific base because their editing windows include multiple base-editing sites, and they are particularly useful as single-base editing tools in the fields such as gene therapy and construction of animal and plant models.

In some embodiments, the present disclosure provides use of the gRNA mutant or base editor as a medication or in the preparation of a medication for gene therapy. Because of having a narrowed editing window; the gRNA mutant or base editor could realize specific editing of one base and avoid side effects caused by additional gene editing, and it is suitable as a gene therapy medication for diseases and has a great prospect of medical applications.

### EXAMPLES

Other purposes, features, and advantages of the present disclosure will become apparent from the following detailed descriptions. It is understood, however, that the detailed descriptions and specific examples (while indicating the specific embodiments of the present disclosure) are provided by way of illustration only because various changes and modifications made within the spirit and scope of the present disclosure will become obvious to a person skilled in the art after reading the detailed descriptions.

The present disclosure will be illustrated with reference to the specific examples. The reagents, samples, and the like used in the examples are all commercially available or accessible to the public by other approaches; and they are provided only as instances but are not unique to the present disclosure and may be replaced by other appropriate tools and biological materials, respectively. The experimental procedures involved may be carried out according to the conditions and methods described in Molecular Cloning: A Laboratory Manual (Third Edition) (Science Press, 2002) and may be carried out according to the manufacturers' instructions for commercially available enzymes or kits. Other experimental methods not described in detail herein are all conventional methods known to a person skilled in the art unless otherwise specified. The sequencing and gene synthesis described in the following examples have been conducted in GENEWIZ, Inc.

BE4max (Addgene: 112093) and hy BE4max (Addgene: 157942) base editor plasmids

NG-ABEmax (Addgene: 124163) base editor plasmid and SaABEmax (Addgene: 119814)

### Example 1 Editing by CBE with Modified gRNA

In mammalian cells, different editing sites were edited by cytosine base editors with igRNAs, and the percentages of base editing of single-window were increased by up to 38.50 folds compared to the case of using unmodified gRNAs.

Experimental process: HEK293T or Hela cells were plated in a 24-well plate at 5×10⁵ cells/well. When the cells in each well grew to 40% to 60% confluency, two representative cytosine base editor plasmids BE4max and hyBE4max, which were commonly used at present, were transfected with Lipofectamine 2000 (Life, Invitrogen, 11668019) reagents into HEK293T or Hela, together with gRNA plasmids targeted to different editing sites and igRNA plasmids modified with insertions, deletions or mismatches at the amounts of 600 ng of editor plasmids and 300 ng of gRNA/igRNA plasmids, respectively. Transfection of each combination of plasmids was performed in triplicate. After 24 h of transfection, 5 μg/ml of puromycin (Merck, USA) was added to the medium. After 120 h of transfection, QuickExtract DNA Extraction Solution (Epicentre, USA) was used to extract genomic DNA. The regions of 200 bp to 300 bp across the edited sites were amplified by PCR using the Taq DNA polymerase (CWBIO, China). The PCR products were subjected to high-throughput sequencing to calculate the editing efficiency (GENEWIZ, China).

Experimental results: Five different genomic loci NSD1, DNMT3B, RNF2, RNF216, and NSD1 were selected. At each locus, gRNA perfectly matching the editing sites and 3 to 5 igRNAs with insertions, deletions or mismatches compared to the editing sites were selected for editing. The results showed that at the NSD1 locus of HEK293T, when BE4max and igRNA (with a deletion of C at the third position) were used, the C-to-T editing efficiency of single C at the sixth position (C6) at the editing sites was increased from 3.34±0.29% to 34.89±0.23%, and the editing specificity (namely, the ratio of editing of the single C at the sixth position to all kinds of editing) was improved from 8.40±0.66% to 71.70±0.85%; and when hyBE4max and igRNA (with a deletion of C at the third position) were used, the C-to-T editing efficiency and editing specificity of single C at the sixth position (C6) at the editing sites were both improved. In case of using different igRNAs, the editing specificities and editing efficiencies of single C at C8 of DNMT3B, C6 of RNF2, C5 of RNF216, and C6 of NSD1 were all improved to some extent. The details were shown in Table 1 to Table 4.

TABLE 1
C-to-T Editing Efficiency of Cytosine Editor (BE4max) (%)

| | gRNAs/igRNAs | | C3 | C6 | C3&C6 | Others | |
|---|---|---|---|---|---|---|---|
| HIRA | gRNA | Control: | 0.13 ± 0.01 | 3.34 ± 0.29 | 32.70 ± 0.71 | 3.62 ± 0.07 | SEQ ID NO: |
| | | GTCATCTTTACC | | | | | 1 |
| | | CCAGAGCG | | | | | |
| HEK 293T | igRNA1 | Mismatch 1: | 0.07 ± 0.00 | 19.72 ± 0.81 | 12.45 ± 0.14 | 3.52 ± 0.01 | SEQ ID NO: |
| | | GTtATCTTTACC | | | | | 2 |
| | | CCAGAGCG | | | | | |
| | igRNA2 | Mismatch 2: | 0.11 ± 0.01 | 14.91 ± 0.65 | 11.32 ± 0.96 | 2.54 ± 0.41 | SEQ ID NO: |
| | | GTCATgTTTACC | | | | | 3 |
| | | CCAGAGCG | | | | | |
| | igRNA3 | Deletion: | 0.11 ± 0.01 | 34.89 ± 0.23 | 1.64 ± 0.35 | 1.09 ± 0.11 | SEQ ID NO: |
| | | GTATCTTTACCC | | | | | 4 |
| | | CAGAGCG | | | | | |
| | igRNA4 | Insertion: | 0.12 ± 0.02 | 28.14 ± 0.83 | 1.04 ± 0.07 | 1.28 ± 0.04 | SEQ ID NO: |
| | | GTgaCATCTTTA | | | | | 5 |
| | | CCCCAGAGCG | | | | | |
| | | | C5 | C8 | C5&C8 | Others | |
| DNMT3B | gRNA | Control: | 0.32 ± 0.01 | 1.32 ± 0.08 | 61.01 ± 3.32 | 7.03 ± 0.39 | SEQ ID NO: |
| HEK 293T | | GACACGTCTGT | | | | | 6 |
| | | GTAGTGCAC | | | | | |
| | igRNA1 | Mismatch 1: | 0.31 ± 0.01 | 22.10 ± 0.42 | 41.94 ± 0.38 | 6.95 ± 0.07 | SEQ ID NO: |
| | | GAtACGTCTGTG | | | | | 7 |
| | | TAGTGCAC | | | | | |
| | igRNA2 | Mismatch 2: | 0.07 ± 0.01 | 42.92 ± 0.55 | 6.13 ± 0.17 | 8.38 ± 0.29 | SEQ ID NO: |
| | | GttACGTCTGTG | | | | | 8 |
| | | TAGTGCAC | | | | | |
| | igRNA3 | Deletion: | 0.22 ± 0.03 | 76.98 ± 1.19 | 0.56 ± 0.08 | 0.74 ± 0.07 | SEQ ID NO: |
| | | GCACGTCTGTG | | | | | 9 |
| | | TAGTGCAC | | | | | |
| | igRNA4 | Insertion: | 0.26 ± 0.05 | 65.51 ± 0.37 | 1.33 ± 0.04 | 0.96 ± 0.07 | SEQ ID NO: |
| | | GAgtCACGTCTG | | | | | 10 |
| | | TGTAGTGCAC | | | | | |
| | | | C3 | C6 | C3&C6 | Others | |
| RNF2 | gRNA | Control: | 0.12 ± 0.03 | 5.03 ± 0.08 | 40.62 ± 0.58 | 10.79 ± 0.24 | SEQ ID NO: |
| | | GTCATCTTAGTC | | | | | 11 |
| | | ATTACCTG | | | | | |
| HEK 293T | igRNA1 | Mismatch: | 0.04 ± 0.01 | 26.03 ± 0.62 | 14.44 ± 0.32 | 7.23 ± 0.17 | SEQ ID NO: |
| | | GTtATCTTAGTC | | | | | 12 |
| | | ATTACCTG | | | | | |
| | igRNA2 | Deletion: | 0.08 ± 0.01 | 20.81 ± 0.28 | 17.52 ± 0.42 | 9.10 ± 0.34 | SEQ ID NO: |
| | | GCATCTTAGTC | | | | | 13 |
| | | ATTACCTG | | | | | |
| | igRNA3 | Insertion: | 0.10 ± 0.01 | 43.16 ± 0.81 | 5.04 ± 0.22 | 2.97 ± 0.22 | SEQ ID NO: |
| | | GTaCATCTTAGT | | | | | 14 |
| | | CATTACCTG | | | | | |
| | | | C5 | C6 | C5&C6 | Others | |
| RNF216 | gRNA | Control: | 0.49 ± 0.02 | 0.16 ± 0.01 | 64.46 ± 0.72 | 5.35 ± 0.09 | SEQ ID NO: |
| | | GTGTCCTTTGA | | | | | 15 |
| | | GCTCGTGCA | | | | | |
| HEK 293T | igRNA1 | Mismatch: | 1.28 ± 0.06 | 0.15 ± 0.01 | 63.69 ± 0.52 | 5.63 ± 0.19 | SEQ ID NO: |
| | | GTGTCtTTTGAG | | | | | 16 |
| | | CTCGTGCA | | | | | |
| | igRNA2 | Deletion: | 1.53 ± 0.04 | 0.15 ± 0.01 | 63.31 ± 0.04 | 5.61 ± 0.08 | SEQ ID NO: |
| | | GTTCCTTTGAG | | | | | 17 |
| | | CTCGTGCA | | | | | |
| | igRNA3 | Insertion: | 1.36 ± 0.04 | 0.16 ± 0.01 | 64.31 ± 0.05 | 5.46 ± 0.21 | SEQ ID NO: |
| | | GTtGTCCTTTGA | | | | | 18 |
| | | GCTCGTGCA | | | | | |
| | | | C3 | C6 | C3&C6 | Others | |
| NSD1 | gRNA | Control: | 0 | 52.49 ± 0.06 | 1.94 ± 0.04 | 4.17 ± 0.14 | SEQ ID NO: |
| | | GGCATCAGTGT | | | | | 19 |
| | | GACATCTGC | | | | | |
| HEK 293T | igRNA1 | Mismatch: | 0 | 59.34 ± 0.18 | 0.13 ± 0.02 | 2.95 ± 0.06 | SEQ ID NO: |
| | | GGtATCAGTGTG | | | | | 20 |
| | | ACATCTGC | | | | | |
| | igRNA2 | Deletion: | 0 | 55.90 ± 0.28 | 0.26 ± .04 | 5.26 ± 0.38 | SEQ ID NO: |
| | | GATCAGTGTGA | | | | | 21 |
| | | CATCTGC | | | | | |
| | igRNA3 | Insertion: | 0 | 56.45 ± 0.48 | 0.18 ± 0.04 | 6.17 ± 0.35 | SEQ ID NO: |
| | | GtGCATCAGTGT | | | | | 22 |
| | | GACATCTGC | | | | | |
| | | | C5 | C8 | C5 & C8 | Others | |
| DNMT3B | gRNA | Control: | 1.13 ± 0.28 | 2.49 ± 0.14 | 72.39 ± 1.63 | 8.33 ± 0.22 | SEQ ID NO: |
| | | GACACGTCTGT | | | | | 6 |
| | | GTAGTGCAC | | | | | |
| Hela | igRNA1 | Mismatch 1: | 0.57 ± 0.41 | 29.30 ± 0.79 | 47.34 ± 2.49 | 6.55 ± 1.05 | SEQ ID NO: |
| | | GAtACGTCTGTG | | | | | 7 |
| | | TAGTGCAC | | | | | |
| | igRNA2 | Mismatch 2: | 0.26 ± 0.09 | 63.29 ± 3.17 | 8.14 ± 1.62 | 3.67 ± 0.99 | SEQ ID NO: |
| | | GttACGTCTGTG | | | | | 8 |
| | | TAGTGCAC | | | | | |
| | igRNA3 | Deletion: | 0.23 ± 0.11 | 72.14 ± 1.87 | 2.31 ± 0.32 | 0.97 ± 0.13 | SEQ ID NO: |
| | | GCACGTCTGTG | | | | | 9 |
| | | TAGTGCAC | | | | | |
| | igRNA4 | Insertion: | 0.19 ± 0.03 | 70.58 ± 0.11 | 2.89 ± 0.64 | 0.63 ± 0.21 | SEQ ID NO: |
| | | GAgtCACGTCTG | | | | | 10 |
| | | TGTAGTGCAC | | | | | |

TABLE 2
C-to-T Editing Efficiency of Cytosine Editor (hyBE4max) (%)

| | gRNAs/igRNAs | | C3 | C6 | C3&C6 | Others | |
|---|---|---|---|---|---|---|---|
| NSD1 | gRNA | Control: | 0.08 ± 0.01 | 5.35 ± 0.34 | 19.51 ± 0.57 | 12.65 ± 0.42 | SEQ ID |
| | | GTCATCTTTACC | | | | | NO: 1 |
| | | CCAGAGCG | | | | | |
| HEK 293T | igRNA1 | Mismatch 1: | 0.04 ± 0.01 | 18.57 ± 0.26 | 4.09 ± 0.07 | 11.13 ± 0.35 | SEQ ID |
| | | GTtATCTTTACC | | | | | NO: 2 |
| | | CCAGAGCG | | | | | |
| | igRNA2 | Mismatch 2: | 0.04 ± 0.00 | 11.93 ± 0.55 | 4.67 ± 0.32 | 6.02 ± 0.45 | SEQ ID |
| | | GTCATgTTTACC | | | | | NO: 3 |
| | | CCAGAGCG | | | | | |
| | igRNA3 | Deletion: | 0.05 ± 0.00 | 28.03 ± 0.28 | 2.26 ± 0.21 | 3.28 ± 0.25 | SEQ ID |
| | | GTATCTTTACCC | | | | | NO: 4 |
| | | CAGAGCG | | | | | |
| | igRNA4 | Insertion: | 0.05 ± 0.01 | 34.80 ± 1.25 | 1.72 ± 0.19 | 2.33 ± 0.12 | SEQ ID |
| | | GTgaCATCTTTA | | | | | NO: 5 |
| | | CCCCAGAGCG | | | | | |
| | | | C5 | C8 | C5&C8 | Others | |
| DNMT3B | gRNA | Control: | 0.29 ± 0.02 | 2.85 ± 0.07 | 59.48 ± 0.92 | 6.40 ± 0.07 | SEQ ID |
| HEK 293T | | GACACGTCTGT | | | | | NO: 6 |
| | | GTAGTGCAC | | | | | |
| | igRNA1 | Mismatch 1: | 0.15 ± 0.01 | 35.44 ± 0.31 | 24.16 ± 0.16 | 3.98 ± 0.03 | SEQ ID |
| | | GAtACGTCTGTG | | | | | NO: 7 |
| | | TAGTGCAC | | | | | |
| | igRNA2 | Mismatch 2: | 0.05 ± 0.00 | 49.70 ± 1.03 | 5.73 ± 0.03 | 3.52 ± 0.32 | SEQ ID |
| | | GttACGTCTGTG | | | | | NO: 8 |
| | | TAGTGCAC | | | | | |
| | igRNA3 | Deletion: | 0.19 ± 0.01 | 60.93 ± 0.30 | 2.37 ± 0.19 | 1.65 ± 0.24 | SEQ ID |
| | | GCACGTCTGTG | | | | | NO: 9 |
| | | TAGTGCAC | | | | | |
| | igRNA4 | Insertion: | 0.17 ± 0.01 | 68.95 ± 0.13 | 1.46 ± 0.31 | 0.84 ± 0.07 | SEQ ID |
| | | GAgtCACGTCTG | | | | | NO: 10 |
| | | TGTAGTGCAC | | | | | |
| | | | C3 | C6 | C3&C6 | Others | |
| RNF2 | gRNA | Control: | 0.11 ± 0.01 | 8.60 ± 0.14 | 24.07 ± 0.14 | 15.97 ± 0.27 | SEQ ID |
| | | GTCATCTTAGTC | | | | | NO: 11 |
| | | ATTACCTG | | | | | |
| HEK 293T | igRNA1 | Mismatch: | 0.03 ± 0.02 | 19.37 ± 0.05 | 4.78 ± 0.12 | 10.14 ± 0.27 | SEQ ID |
| | | GTtATCTTAGTC | | | | | NO: 12 |
| | | ATTACCTG | | | | | |
| | igRNA2 | Deletion: | 0.04 ± 0.01 | 25.89 ± 0.34 | 2.45 ± 0.31 | 5.02 ± 0.14 | SEQ ID |
| | | GCATCTTAGTC | | | | | NO: 13 |
| | | ATTACCTG | | | | | |
| | igRNA3 | Insertion: | 0.03 ± 0.01 | 25.24 ± 0.30 | 2.09 ± 0.20 | 3.96 ± 0.27 | SEQ ID |
| | | GTaCATCTTAGT | | | | | NO: 14 |
| | | CATTACCTG | | | | | |
| | | | C5 | C6 | C5&C6 | Others | |
| RNF216 | ?RNA | Control: | 0.55 ± 0.12 | 0.14 ± 0.02 | 41.98 ± 7.15 | 18.69 ± 8.47 | SEQ ID |
| | | GTGTCCTTTGA | | | | | NO: 15 |
| | | GCTCGTGCA | | | | | |
| HEK 293T | igRNA1 | Mismatch: | 2.49 ± 0.13 | 0.15 ± 0.01 | 53.50 ± 0.64 | 6.90 ± 0.02 | SEQ ID |
| | | GTGTCtTTTGAG | | | | | NO: 16 |
| | | CTCGTGCA | | | | | |
| | igRNA2 | Deletion: | 2.57 ± 0.14 | 0.15 ± 0.00 | 49.74 ± 0.13 | 7.41 ± 0.13 | SEQ ID |
| | | GTTCCTTTGAG | | | | | NO: 17 |
| | | CTCGTGCA | | | | | |
| | igRNA3 | Insertion: | 1.93 ± 0.10 | 0.18 ± 0.01 | 46.31 ± 0.29 | 7.95 ± 0.08 | SEQ ID |
| | | GTtGTCCTTTGA | | | | | NO: 18 |
| | | GCTCGTGCA | | | | | |
| | | | C3 | C6 | C3&C6 | Others | |
| NSD1 | gRNA | Control: | 0 | 54.19 ± 1.44 | 0.54 ± 0.07 | 3.96 ± 0.32 | SEQ ID |
| | | GGCATCAGTGT | | | | | NO: 19 |
| | | GACATCTGC | | | | | |
| HEK 293T | igRNA1 | Mismatch: | 0.01 ± 0.02 | 47.03 ± 0.60 | 0.05 ± 0.00 | 2.44 ± 0.09 | SEQ ID |
| | | GGtATCAGTGTG | | | | | NO: 20 |
| | | ACATCTGC | | | | | |
| | igRNA2 | Deletion: | 0.01 ± 0.01 | 47.01 ± 1.31 | 0.10 ± 0.01 | 3.09 ± 0.16 | SEQ ID |
| | | GATCAGTGTGA | | | | | NO: 21 |
| | | CATCTGC | | | | | |
| | igRNA3 | Insertion: | 0 | 42.78 ± 0.78 | 0.07 ± 0.01 | 3.35 ± 0.22 | SEQ ID |
| | | GtGCATCAGTGT | | | | | NO: 22 |
| | | GACATCTGC | | | | | |

TABLE 3
C-to-T Editing Specificity of Cytosine Editor (BE4max) (%)

| | gRNAs/igRNAs | | C3 | C6 | C3&C6 | Others | |
|---|---|---|---|---|---|---|---|
| HIRA | gRNA | Control: | 0.32 ± 0.01 | 8.40 ± 0.66 | 82.17 ± 0.54 | 9.11 ± 0.14 | SEQ ID |
| | | GTCATCTTTACC | | | | | NO: 1 |
| | | CCAGAGCG | | | | | |
| HEK 293T | igRNA1 | Mismatch 1: | 0.20 ± 0.01 | 55.75 ± 0.62 | 34.17 ± 0.76 | 9.88 ± 0.36 | SEQ ID |
| | | GTtATCTTTACC | | | | | NO: 2 |
| | | CCAGAGCG | | | | | |
| | igRNA2 | Mismatch 2: | 0.39 ± 0.05 | 51.68 ± 2.88 | 39.16 ± 2.72 | 8.77 ± 1.18 | SEQ ID |
| | | GTCATgTTTACC | | | | | NO: 3 |
| | | CCAGAGCG | | | | | |
| | igRNA3 | Deletion: | 0.33 ± 0.03 | 71.70 ± 0.85 | 24.84 ± 0.88 | 3.14 ± 0.33 | SEQ ID |
| | | GTATCTTTACCC | | | | | NO: 4 |
| | | CAGAGCG | | | | | |
| | igRNA4 | Insertion: | 0.33 ± 0.06 | 76.92 ± 0.55 | 19.27 ± 0.59 | 3.49 ± 0.05 | SEQ ID |
| | | GTgaCATCTTTA | | | | | NO: 5 |
| | | CCCCAGAGCG | | | | | |
| | | | C5 | C8 | C5&C8 | Others | |
| DNMT3B | gRNA | Control: | 0.46 ± 0.02 | .89 ± 0.07 | 87.57 ± 0.12 | 10.08-0.04 | SEQ ID |
| | | GACACGTCTGT | | | | | NO: 6 |
| | | GTAGTGCAC | | | | | |
| HEK 293T | igRNA1 | Mismatch 1: | 0.44 ± 0.01 | 31.00 ± 0.26 | 58.82 ± 0.28 | 9.74 ± 0.12 | SEQ ID |
| | | GAtACGTCTGTG | | | | | NO: 7 |
| | | TAGTGCAC | | | | | |
| | igRNA2 | Mismatch 2: | 0.11 ± 0.01 | 74.66 ± 0.44 | 10.65 ± 0.16 | 14.57 ± 0.50 | SEQ ID |
| | | GttACGTCTGTG | | | | | NO: 8 |
| | | TAGTGCAC | | | | | |
| | igRNA3 | Deletion: | 0.38 ± 0.04 | 80.77 ± 0.28 | 7.85 ± 0.35 | 10.99 ± 0.58 | SEQ ID |
| | | GCACGTCTGTG | | | | | NO: 9 |
| | | TAGTGCAC | | | | | |
| | igRNA4 | Insertion: | 0.39 ± 0.08 | 85.32 ± 0.17 | 6.66 ± 0.05 | 7.62 ± 0.13 | SEQ ID |
| | | GAgtCACGTCTG | | | | | NO: 10 |
| | | TGTAGTGCAC | | | | | |
| | | | C3 | C6 | C3&C6 | Others | |
| RNF2 | gRNA | Control: | 0.21 ± 0.06 | .89 ± 0.17 | 71.82 ± 0.55 | 19.08 ± 0.35 | SEQ ID |
| HEK 293T | | GTCATCTTAGTC | | | | | NO: 11 |
| | | ATTACCTG | | | | | |
| | igRNA1 | Mismatch: | 0.09 ± 0.03 | 54.52 ± 0.27 | 30.24 ± 0.06 | 15.15 ± 0.32 | SEQ ID |
| | | GTtATCTTAGTC | | | | | NO: 12 |
| | | ATTACCTG | | | | | |
| | igRNA2 | Deletion: | 0.16 ± 0.02 | 43.80 ± 0.65 | 36.88 ± 0.86 | 19.16 ± 0.70 | SEQ ID |
| | | GCATCTTAGTC | | | | | NO: 13 |
| | | ATTACCTG | | | | | |
| | igRNA3 | Insertion: | 0.20 ± 0.02 | 67.3 ± 41.44 | 22.40 ± 0.53 | 10.05 ± 0.93 | SEQ ID |
| | | GTaCATCTTAGT | | | | | NO: 14 |
| | | CATTACCTG | | | | | |
| | | | C5 | C6 | C5&C6 | Others | |
| RNF216 | gRNA | Control: | 0.70 ± 0.03 | .23 ± 0.01 | 91.49 ± 0.18 | 7.59 ± 0.18 | SEQ ID |
| | | GTGTCCTTTGA | | | | | NO: 15 |
| | | GCTCGTGCA | | | | | |
| HEK 293T | igRNA1 | Mismatch: | 1.81 ± 0.09 | .21 ± 0.01 | 90.02 ± 0.16 | 7.96 ± 0.24 | SEQ ID |
| | | GTGTCtTTTGAG | | | | | NO: 16 |
| | | CTCGTGCA | | | | | |
| | igRNA2 | Deletion: | 2.16 ± 0.05 | .21 ± 0.01 | 89.68 ± 0.13 | 7.95 ± 0.10 | SEQ ID |
| | | GTTCCTTTGAG | | | | | NO: 17 |
| | | CTCGTGCA | | | | | |
| | igRNA3 | Insertion: | 1.91 ± 0.05 | .23 ± 0.01 | 90.20 ± 0.29 | 7.66 ± 0.27 | SEQ ID |
| | | GTtGTCCTTTGA | | | | | NO: 18 |
| | | GCTCGTGCA | | | | | |
| | | | C3 | C6 | C3&C6 | Others | |
| NSD1 | gRNA | Control: | 0 | 89.58 ± 0.27 | .31 ± 0.06 | 7.11 ± 0.21 | SEQ ID |
| | | GGCATCAGTGT | | | | | NO: 19 |
| | | GACATCTGC | | | | | |
| HEK 293T | igRNA1 | Mismatch: | 0 | 95.06 ± 0.11 | .21 ± 0.03 | 4.73 ± 0.08 | SEQ ID |
| | | GGtATCAGTGTG | | | | | NO: 20 |
| | | ACATCTGC | | | | | |
| | igRNA2 | Deletion: | 0 | 91.03 ± 0.58 | .42 ± 0.07 | 8.56 ± 0.57 | SEQ ID |
| | | GATCAGTGTGA | | | | | NO: 21 |
| | | CATCTGC | | | | | |
| | igRNA3 | Insertion: | 0 | 89.89 ± 0.45 | .29 ± 0.06 | 9.83 ± 0.46 | SEQ ID |
| | | GtGCATCAGTGT | | | | | NO: 22 |
| | | GACATCTGC | | | | | |
| | | | C5 | C8 | C5 & C8 | Others | |
| DNMT3B | gRNA | Control: | 1.34 ± 0.22 | .95 ± 0.63 | 85.83 ± 1.14 | 9.88 ± 0.96 | SEQ ID |
| | | GACACGTCTGT | | | | | NO: 6 |
| | | GTAGTGCAC | | | | | |
| Hela | igRNA1 | Mismatch 1: | 0.68 ± 0.12 | 34.98 ± 0.24 | 56.52 ± 0.41 | 7.82 ± 0.36 | SEQ ID |
| | | GAtACGTCTGTG | | | | | NO: 7 |
| | | TAGTGCAC | | | | | |
| | igRNA2 | Mismatch 2: | 0.35 ± 0.03 | 83.98 ± 2.10 | 10.80 ± 0.26 | 4.87 ± 0.33 | SEQ ID |
| | | GttACGTCTGTG | | | | | NO: 8 |
| | | TAGTGCAC | | | | | |
| | igRNA3 | Deletion: | 0.30 ± 0.14 | 95.36 ± 0.41 | .05 ± 0.74 | 1.28 ± 0.11 | SEQ ID |
| | | GCACGTCTGTG | | | | | NO: 9 |
| | | TAGTGCAC | | | | | |
| | igRNA4 | Insertion: | 0.26 ± 0.19 | 95.01 ± 1.62 | .89 ± 0.22 | 0.85 ± 0.96 | SEQ ID |
| | | GAgtCACGTCTG | | | | | NO: 10 |
| | | TGTAGTGCAC | | | | | |

TABLE 4
C-to-T Editing Specificity of Cytosine Editor (hyBE4max) (%)

| | gRNAs/igRNAs | | C3 | C6 | C3&C6 | Others | |
|---|---|---|---|---|---|---|---|
| HIRA | gRNA | Control: | 0.21 ± 0.03 | 14.22 ± 0.39 | 51.91 ± 0.33 | 33.66 ± 0.09 | SEQ ID |
| | | GTCATCTTTACC | | | | | NO: 1 |
| | | CCAGAGCG | | | | | |
| HEK 293T | igRNA1 | Mismatch 1: | 0.11 ± 0.01 | 54.90 ± 0.50 | 12.08 ± 0.37 | 32.91 ± 0.70 | SEQ ID |
| | | GTtATCTTTACC | | | | | NO: 2 |
| | | CCAGAGCG | | | | | |
| | igRNA2 | Mismatch 2: | 0.16 ± 0.02 | 52.69 ± 0.93 | 20.61 ± 0.50 | 26.53 ± 0.49 | SEQ ID |
| | | GTCATgTTTACC | | | | | NO: 3 |
| | | CCAGAGCG | | | | | |
| | igRNA3 | Deletion: | 0.16 ± 0.01 | 70.19 ± 0.40 | 11.79 ± 0.85 | 17.86 ± 0.63 | SEQ ID |
| | | GTATCTTTACCC | | | | | NO: 4 |
| | | CAGAGCG | | | | | |
| | igRNA4 | Insertion: | 0.15 ± 0.04 | 70.34 ± 2.00 | 13.41 ± 0.80 | 16.09 ± 1.20 | SEQ ID |
| | | GTgaCATCTTTA | | | | | NO: 5 |
| | | CCCCAGAGCG | | | | | |
| | | | C5 | C8 | C5&C8 | Others | |
| DNMT3B | gRNA | Control: | 0.43 ± 0.04 | 4.13 ± 0.14 | 86.17 ± 0.23 | 9.27 ± 0.09 | SEQ ID |
| | | GACACGTCTGT | | | | | NO: 6 |
| | | GTAGTGCAC | | | | | |
| HEK 293T | igRNA1 | Mismatch 1: | 0.24 ± 0.01 | 55.61 ± 0.17 | 37.91 ± 0.16 | 6.25 ± 0.07 | SEQ ID |
| | | GAtACGTCTGTG | | | | | NO: 7 |
| | | TAGTGCAC | | | | | |
| | igRNA2 | Mismatch 2: | 0.08 ± 0.00 | 84.24 ± 0.59 | 9.71 ± 0.12 | 5.98 ± 0.57 | SEQ ID |
| | | GttACGTCTGTG | | | | | NO: 8 |
| | | TAGTGCAC | | | | | |
| | igRNA3 | Deletion: | 0.32 ± 0.02 | 84.12 ± 0.55 | 7.38 ± 0.30 | 6.18 ± 0.41 | SEQ ID |
| | | GCACGTCTGTG | | | | | NO: 9 |
| | | TAGTGCAC | | | | | |
| | igRNA4 | Insertion: | 0.26 ± 0.02 | 87.00 ± 0.61 | 7.07 ± 0.71 | 5.67 ± 0.12 | SEQ ID |
| | | GAgtCACGTCTG | | | | | NO: 10 |
| | | TGTAGTGCAC | | | | | |
| | | | C3 | C6 | C3&C6 | Others | |
| RNF2 | gRNA | Control: | 0.23 ± 0.02 | 17.63 ± 0.19 | 49.37 ± 0.30 | 32.76 ± 0.23 | SEQ ID |
| HEK 293T | | GTCATCTTAGTC | | | | | NO: 11 |
| | | ATTACCTG | | | | | |
| | igRNA1 | Mismatch: | 0.09 ± 0.04 | 56.45 ± 0.55 | 13.93 ± 0.27 | 29.52 ± 0.50 | SEQ ID |
| | | GTtATCTTAGTC | | | | | NO: 12 |
| | | ATTACCTG | | | | | |
| | igRNA2 | Deletion: | 0.13 ± 0.04 | 77.53 ± 1.25 | 7.32 ± 0.91 | 15.02 ± 0.38 | SEQ ID |
| | | GCATCTTAGTC | | | | | NO: 13 |
| | | ATTACCTG | | | | | |
| | igRNA3 | Insertion: | 0.10 ± 0.03 | 80.57 ± 0.96 | 6.68 ± 0.61 | 12.65 ± 0.88 | SEQ ID |
| | | GTaCATCTTAGT | | | | | NO: 14 |
| | | CATTACCTG | | | | | |
| | | | C5 | C6 | C5&C6 | Others | |
| RNF216 | gRNA | Control: | 0.90 ± 0.20 | 0.22 ± 0.03 | 68.69 ± 13.2 | 30.19 ± 13.42 | SEQ ID |
| | | GTGTCCTTTGA | | | | | NO: 15 |
| | | GCTCGTGCA | | | | | |
| HEK 293T | igRNA1 | Mismatch: | 3.95 ± 0.23 | 0.24 ± 0.01 | 84.86 ± 0.30 | 10.95 ± 0.10 | SEQ ID |
| | | GTGTCtTTTGAG | | | | | NO: 16 |
| | | CTCGTGCA | | | | | |
| | igRNA2 | Deletion: | 4.30 ± 0.22 | 0.24 ± 0.01 | 83.08 ± 0.34 | 12.38 ± 0.18 | SEQ ID |
| | | GTTCCTTTGAG | | | | | NO: 17 |
| | | CTCGTGCA | | | | | |
| | igRNA3 | Insertion: | 3.42 ± 0.15 | 0.31 ± 0.02 | 82.17 ± 0.11 | 14.10 ± 0.20 | SEQ ID |
| | | GTtGTCCTTTGA | | | | | NO: 18 |
| | | GCTCGTGCA | | | | | |
| | | | C3 | C6 | C3&C6 | Others | |
| NSD1 | gRNA | Control: | 0 | 92.35 ± 0.43 | 0.92 ± 0.09 | 6.73 ± 0.33 | SEQ ID |
| | | GGCATCAGTGT | | | | | NO: 19 |
| | | GACATCTGC | | | | | |
| HEK 293T | igRNA1 | Mismatch: | 0.02 ± 0.03 | 94.94 ± 0.16 | 0.11 ± 0.01 | 4.92 ± 0.15 | SEQ ID |
| | | GGtATCAGTGTG | | | | | NO: 20 |
| | | ACATCTGC | | | | | |
| | igRNA2 | Deletion: | 0.01 ± 0.02 | 93.62 ± 0.43 | 0.19 ± 0.02 | 6.17 ± 0.42 | SEQ ID |
| | | GATCAGTGTGA | | | | | NO: 21 |
| | | CATCTGC | | | | | |
| | igRNA3 | Insertion: | 0 | 92.58 ± 0.50 | 0.16 ± 0.03 | 7.26 ± 0.47 | SEQ ID |
| | | GtGCATCAGTGT | | | | | NO: 22 |
| | | GACATCTGC | | | | | |

### Example 2 Editing by ABE with Modified gRNA

In mammalian cells, different editing sites were edited by adenine base editors using modified gRNA, and the percentages of base editing of single-window were increased by up to 10.15 folds compared to the case of using unmodified gRNAs.

Experimental process: HEK293T or Hela cells were plated in a 24-well plate at 5×10⁵ cells/well. When the cells in each well grew to 40% to 60% confluency, a representative adenine base editor NG-ABEmax plasmid, which was commonly used at present, was transfected with the Lipofectamine 2000 (Life, Invitrogen, 11668019) reagent into HEK293T or Hela, together with gRNA plasmids targeted to different editing sites and igRNA plasmids modified with insertions, deletions or mismatches at the amounts of 600 ng of editor plasmids and 300 ng of gRNA/igRNA plasmids, respectively. Transfection of each combination of plasmids was performed in triplicate. After 24 h of transfection, 5 μg/ml of puromycin (Merck, USA) was added to the medium. After 120 h of transfection, QuickExtract DNA Extraction Solution (Epicentre, USA) was used to extract genomic DNA. The regions of 200 bp to 300 bp across the edited sites were amplified by PCR using the Taq DNA polymerase (CWBIO, China). The PCR products were subjected to high-throughput sequencing to calculate the editing efficiency (GENEWIZ, China).

Experimental results: Nine different genomic loci PSMB2, ABCA3, EMX1-SITE3, VISTA hs267, SNCA, ANO5, KCNQ2, NOTCH2, and GFI1 were selected. At each locus, gRNA perfectly matching the editing sites and 3 to 5 igRNAs with insertions, deletions, mismatches compared to the editing sites were selected for editing.

The results showed that at the ANO5 locus of HEK293T, when NG-ABEmax and igRNA (with base A inserted at the third position) were used, the A-to-G editing specificity of single A at the seventh position (A7) at the editing sites was improved from 27.75±0.03% to 91.34±0.94%, and the editing efficiency was increased from 15.46±0.66% to 24.94±0.59%. In case of using different igRNAs, the editing specificities and editing efficiencies of single A at A5 of PSMB2, A5 of ABCA3, A6 of EMX1-SITE3, A5 of VISTA hs267, A5 of SNCA, A5 of KCNQ2, A5 of NOTCH2, and A5 of GFI1 were all improved accordingly. The details were shown in Table 5 and Table 6.

TABLE 5
A-to-G Editing Efficiency of Adenine Editor (%)

| Genes | gRNAs/igRNAs | | NG.ABEmax | | | | |
|---|---|---|---|---|---|---|---|
| | | | A5 | A7 | A5&A7 | Others | |
| PSMB2 | gRNA | Control: | 44.22 ± 0.97 | 0.47 ± 0.05 | 12.94 ± 0.55 | 12.03 ± 0.92 | SEQ ID |
| (HEK | | GTAAACAAAGCAT | | | | | NO: 23 |
| 293T) | | AGACTGA | | | | | |
| | igRNA1 | Mismatch 1: | 27.42 ± 0.91 | 1.95 ± 0.05 | 1.46 ± 0.13 | 3.26 ± 0.12 | SEQ ID |
| | | GTtAACAAAGCAT | | | | | NO: 24 |
| | | AGACTGA | | | | | |
| | igRNA2 | Mismatch 2: | 40.66 ± 4.04 | 2.23 ± 0.25 | 4.73 ± 0.46 | 4.92 ± 0.54 | SEQ ID |
| | | GTAAtCAAAGCAT | | | | | NO: 25 |
| | | AGACTGA | | | | | |
| | igRNA3 | Deletion: | 29.44 ± 0.60 | 2.15 ± 0.38 | 1.29 ± 0.07 | 3.11 ± 0.10 | SEQ ID |
| | | GTAACAAAGCATA | | | | | NO: 26 |
| | | GACTGA | | | | | |
| | igRNA4 | Insertion: | 27.01 ± .0.74 | 1.13 ± 0.12 | 1.24 ± 0.19 | 2.52 ± 0.42 | SEQ ID |
| | | GTgAAACAAAGC | | | | | NO: 27 |
| | | ATAGACTGA | | | | | |
| | | | A5 | A8 | A5&A8 | Others | |
| ABCA3 | gRNA | Control: | 47.77 ± 0.68 | 0.09 ± 0.01 | 14.42 ± 0.46 | 2.86 ± 0.06 | SEQ ID |
| (HEK | | GAAGAGCAGGGT | | | | | NO: 28 |
| 293T) | | CATGAAGG | | | | | |
| | igRNA1 | Mismatch 1: | 56.72 ± 1.64 | 0.20 ± 0.01 | 7.40 ± 0.24 | 2.51 ± 0.21 | SEQ ID |
| | | GAtGAGCAGGGTC | | | | | NO: 29 |
| | | ATGAAGG | | | | | |
| | igRNA2 | Deletion: | 58.15 ± 1.07 | 0.23 ± 0.09 | 5.14 ± 0.22 | 2.08 ± 0.35 | SEQ ID |
| | | GAGAGCAGGGTC | | | | | NO: 30 |
| | | ATGAAGG | | | | | |
| | igRNA3 | Insertion: | 58.91 ± 1.08 | 0.16 ± 0.05 | 4.88 ± 0.16 | 1.45 ± 0.07 | SEQ ID |
| | | GcAAGAGCAGGG | | | | | NO: 31 |
| | | TCATGAAGG | | | | | |
| | | | A6 | A8 | A6&A8 | Others | |
| EMX1- | gRNA | Control: | 19.54 ± 0.44 | 0.37 ± 0.00 | 10.51 ± 0.23 | 10.12 ± 0.11 | SEQ ID |
| SITE3 | | GGAGCACACATG | | | | | NO: 32 |
| (HEK | | CCCAGGTG | | | | | |
| 293T) | igRNA1 | Mismatch 1: | 27.15 ± 0.29 | 1.19 ± 0.06 | 3.99 ± 0.07 | 5.19 ± 0.06 | SEQ ID |
| | | GGtGCACACATGC | | | | | NO: 33 |
| | | CCAGGTG | | | | | |
| | igRNA2 | Mismatch 2: | 13.71 ± 0.44 | 1.42 ± 0.10 | 1.76 ± 0.08 | 2.51 ± 0.12 | SEQ ID |
| | | GGAcCACACATGC | | | | | NO: 34 |
| | | CCAGGTG | | | | | |
| | igRNA3 | Deletion: | 31.71 ± 1.18 | 0.76 ± 0.15 | 1.24 ± 0.10 | 1.23 ± 0.15 | SEQ ID |
| | | GAGCACACATGCC | | | | | NO: 35 |
| | | CAGGTG | | | | | |
| | igRNA4 | Insertion: | 25.69 ± 1.08 | 1.48 ± 0.15 | 1.86 ± 0.59 | 1.38 ± 0.08 | SEQ ID |
| | | GGtAGCACACATG | | | | | NO: 36 |
| | | CCCAGGTG | | | | | |
| | | | A5 | A7 | A5&A7 | Others | |
| VISTA | gRNA | Control: | 38.10 ± 3.82 | 0.05 ± 0.01 | 19.28 ± 1.74 | 4.16 ± 0.44 | SEQ ID |
| hs267 | | GAACACAAAGCA | | | | | NO: 37 |
| (HEK | | TAGACTGC | | | | | |
| 293T) | igRNA1 | Mismatch 1: | 57.45 ± 0.59 | 0.06 ± 0.00 | 12.20 ± 0.09 | 3.81 ± 0.09 | SEQ ID |
| | | GtACACAAAGCAT | | | | | NO: 38 |
| | | AGACTGC | | | | | |
| | igRNA2 | Mismatch 2: | 64.71 ± 0.85 | 0.09 ± 0.01 | 4.87 ± 0.25 | 4.67 ± 0.14 | SEQ ID |
| | | GAAgACAAAGCAT | | | | | NO: 39 |
| | | AGACTGC | | | | | |
| | igRNA3 | Deletion: | 60.15 ± 1.04 | 0.02 ± 0.01 | 4.31 ± 0.25 | 3.34 ± 0.23 | SEQ ID |
| | | GAAACAAAGCAT | | | | | NO: 40 |
| | | AGACTGC | | | | | |
| | igRNA4 | Insertion: | 69.04 ± 1.04 | 0.05 ± 0.01 | 3.41 ± 0.33 | 3.44 ± 0.32 | SEQ ID |
| | | GAtACACAAAGCA | | | | | NO: 41 |
| | | TAGACTGC | | | | | |
| | | | A5 | A7 | A5&A7 | Others | |
| SNCA | gRNA | Control: | 34.72 ± 1.27 | 0.08 ± 0.00 | 6.33 ± 0.12 | 2.41 ± 0.07 | SEQ ID |
| (HEK | | GAACACAATGCAT | | | | | NO: 42 |
| 293T) | | AGATTGC | | | | | |
| | igRNA1 | Mismatch 1: | 22.61 ± 1.36 | 0.12 ± 0.02 | 2.87 ± 0.14 | 1.76 ± 0.11 | SEQ ID |
| | | GAtCACAATGCAT | | | | | NO: 43 |
| | | AGATTGC | | | | | |
| | igRNA2 | Mismatch 2: | 13.39 ± 0.16 | 0.10 ± 0.00 | 1.80 ± 0.03 | 1.28 ± 0.04 | SEQ ID |
| | | GAACtCAATGCAT | | | | | NO: 44 |
| | | AGATTGC | | | | | |
| | igRNA3 | Deletion: | 20.000.51 | 0.11 ± 0.02 | 1.07 ± 0.07 | 0.84 ± 0.11 | SEQ ID |
| | | GACACAATGCATA | | | | | NO: 45 |
| | | GATTGC | | | | | |
| | igRNA4 | Insertion: | 26.89 ± 0.40 | 0.10 ± 0.02 | 1.10 ± 0.23 | 1.19 ± 0.15 | SEQ ID |
| | | GAcACACAATGCA | | | | | NO: 46 |
| | | TAGATTGC | | | | | |
| | | | A5 | A7 | A5&A7 | Others | |
| ANO5 | gRNA | Control: | 5.72 ± 0.24 | 15.46 ± 0.66 | 27.05 ± 1.07 | 7.49 ± 0.48 | SEQ ID |
| (HEK | | TCACACACTTGAT | | | | | NO: 47 |
| 293T) | | CACAGAG | | | | | |
| | igRNA1 | Mismatch 1: | 1.24 ± 0.07 | 18.56 ± 0.35 | 0.87 ± 0.11 | 1.36 ± 0.12 | SEQ ID |
| | | TgACACACTTGAT | | | | | NO: 48 |
| | | CACAGAG | | | | | |
| | igRNA2 | Mismatch 2: | 1.25 ± 0.04 | 8.80 ± 0.14 | 1.23 ± 0.09 | 1.45 ± 0.06 | SEQ ID |
| | | TCAtACACTTGATC | | | | | NO: 49 |
| | | ACAGAG | | | | | |
| | igRNA3 | Deletion: | 1.74 ± 0.27 | 17.35 ± 0.37 | 0.84 ± 0.15 | 1.20 ± 0.15 | SEQ ID |
| | | TACACACTTGATC | | | | | NO: 50 |
| | | ACAGAG | | | | | |
| | igRNA4 | Insertion: | 0.89 ± 0.07 | 24.94 ± 0.59 | 0.51 ± 0.11 | 0.98 ± 0.16 | SEQ ID |
| | | TCaACACACTTGA | | | | | NO: 51 |
| | | TCACAGAG | | | | | |
| | | | A5 | A6 | A5&A6 | Others | |
| KCNQ2 | gRNA | Control: | 0.79 ± 0.11 | 0.13 ± 0.01 | 47.12 ± 2.69 | 9.15 ± 0.28 | SEQ ID |
| (HEK | | GAAGAAGGAGAC | | | | | NO: 52 |
| 293T) | | ACCGATGA | | | | | |
| | igRNA1 | Mismatch: | 10.41 ± 0.50 | 0.84 ± 0.04 | 50.47 ± 0.57 | 5.39 ± 0.14 | SEQ ID |
| | | GAcGAAGGAGAC | | | | | NO: 53 |
| | | ACCGATGA | | | | | |
| | igRNA2 | Deletion: | 16.18 ± 0.17 | 0.57 ± 0.03 | 38.27 ± 0.96 | 4.06 ± 0.19 | SEQ ID |
| | | GAGAAGGAGACA | | | | | NO: 54 |
| | | CCGATGA | | | | | |
| | igRNA3 | Insertion: | 11.90 ± 0.51 | 0.88 ± 0.21 | 46.24 ± 0.77 | 6.52 ± 0.16 | SEQ ID |
| | | GcAAGAAGGAGA | | | | | NO: 55 |
| | | CACCGATGA | | | | | |
| | | | A5 | A7 | A5&A7 | Others | |
| NOTCH2 | gRNA | Control: | 13.93 ± 0.76 | 2.72 ± 0.18 | 33.64 ± 2.06 | 4.67 ± 0.23 | SEQ ID |
| (HEK | | TGACACAGGAGA | | | | | NO: 56 |
| 293T) | | CCTGTCAC | | | | | |
| | igRNA1 | Mismatch: | 22.74 ± 0.69 | 5.74 ± 0.10 | 22.24 ± 1.10 | 3.54 ± 0.02 | SEQ ID |
| | | TGACAtAGGAGAC | | | | | NO: 57 |
| | | CTGTCAC | | | | | |
| | igRNA2 | Deletion: | 28.92 ± 1.17 | 4.89 ± 0.44 | 15.65 ± 0.27 | 3.32 ± 0.12 | SEQ ID |
| | | TGAACAGGAGAC | | | | | NO: 58 |
| | | CTGTCAC | | | | | |
| | igRNA3 | Insertion: | 2.59 ± 1.32 | 4.00 ± 0.62 | 31.18 ± 0.19 | 4.25 ± 0.10 | SEQ ID |
| | | TGaACACAGGAGA | | | | | NO: 59 |
| | | CCTGTCAC | | | | | |
| | | | A5 | A6 | A5&A6 | Others | |
| GFI1 | gRNA | Control: | 9.57 ± 0.87 | 1.13 ± 0.10 | 30.03 ± 3.06 | 1.32 ± 0.07 | SEQ ID |
| (HEK | | TGGGAAGGGTTTC | | | | | NO: 60 |
| 293T) | | CAGAGGA | | | | | |
| | igRNA1 | Mismatch: | 13.24 ± 1.49 | 2.96 ± 0.20 | 10.25 ± 0.99 | 0.94 ± 0.03 | SEQ ID |
| | | TaGGAAGGGTTTC | | | | | NO: 61 |
| | | CAGAGGA | | | | | |
| | igRNA2 | Deletion: | 15.30 ± 0.24 | 2.04 ± 0.24 | 6.95 ± 0.89 | 0.77 ± 0.08 | SEQ ID |
| | | TGGAAGGGTTTCC | | | | | NO: 62 |
| | | AGAGGA | | | | | |
| | igRNA3 | Insertion: | 18.97 ± 1.23 | 2.13 ± 0.19 | 8.11 ± 0.25 | 0.51 ± 0.09 | SEQ ID |
| | | TGtGGAAGGGTTT | | | | | NO: 63 |
| | | CCAGAGGA | | | | | |
| | | | A5 | A7 | A5 & A7 | Others | |
| ANO5 | gRNA | Control: | 8.33 ± 0.97 | 24.18 ± 1.59 | 33.97 ± 0.16 | 9.23 ± 1.24 | SEQ ID |
| (Hela) | | TCACACACTTGAT | | | | | NO: 47 |
| | | CACAGAG | | | | | |
| | igRNA1 | Mismatch 1: | 2.98 ± 0.16 | 29.08 ± 1.15 | 2.24 ± 0.33 | 1.96 ± 0.58 | SEQ ID |
| | | TgACACACTTGAT | | | | | NO: 48 |
| | | CACAGAG | | | | | |
| | igRNA2 | Mismatch 2: | 2.34 ± 0.24 | 14.30 ± 0.75 | 1.95 ± 0.55 | 2.36 ± 0.21 | SEQ ID |
| | | TCAtACACTTGATC | | | | | NO: 49 |
| | | ACAGAG | | | | | |
| | igRNA3 | Deletion: | 1.76 ± 0.37 | 28.37 ± 0.51 | 1.58 ± 0.31 | 1.77 ± 0.36 | SEQ ID |
| | | TACACACTTGATC | | | | | NO: 50 |
| | | ACAGAG | | | | | |
| | igRNA4 | Insertion: | 1.84 ± 0.29 | 36.32 ± 0.14 | 1.69 ± 0.37 | 1.99 ± 0.27 | SEQ ID |
| | | TCaACACACTTGA | | | | | NO: 51 |
| | | TCACAGAG | | | | | |

TABLE 6
A-to-G Editing Specificity of Adenine Editor (%)

| Genes | gRNAs/igRNAs | | NG.ABEmax | | | | |
|---|---|---|---|---|---|---|---|
| | | | A5 | A7 | A5 & A7 | Others | |
| PSMB2 | gRNA | Control: | 63.49 ± 1.74 | 0.68 ± 0.08 | 18.57 ± 0.63 | 17.26 ± 1.14 | SEQ ID |
| (HEK | | GTAAACAAAGC | | | | | NO: 23 |
| 293T) | | ATAGACTGA | | | | | |
| | igRNA1 | Mismatch 1: | 80.44 ± 0.23 | 5.72 ± 0.29 | 4.26 ± 0.24 | 9.57 ± 0.15 | SEQ ID |
| | | GTtAACAAAGC | | | | | NO: 24 |
| | | ATAGACTGA | | | | | |
| | igRNA2 | Mismatch 2: | 77.40 ± 0.21 | 4.23 ± 0.06 | 9.01 ± 0.05 | 9.36 ± 0.18 | SEQ ID |
| | | GTAAtCAAAGC | | | | | NO: 25 |
| | | ATAGACTGA | | | | | |
| | igRNA3 | Deletion: | 81.81 ± 0.59 | 5.94 ± 0.94 | 3.59 ± 0.21 | 8.66 ± 0.36 | SEQ ID |
| | | GTAACAAAGCA | | | | | NO: 26 |
| | | TAGACTGA | | | | | |
| | igRNA4 | Insertion: | 84.67 ± 0.70 | 3.55 ± 0.32 | 3.87 ± 0.54 | 7.92 ± 1.48 | SEQ ID |
| | | GTgAAACAAAG | | | | | NO: 27 |
| | | CATAGACTGA | | | | | |
| | | | A5 | A8 | A5 & A8 | Others | |
| ABCA3 | gRNA | Control: | 73.33 ± 0.32 | 0.14 ± 0.01 | 22.13 ± 0.33 | 4.40 ± 0.01 | SEQ ID |
| (HEK | | GAAGAGCAGG | | | | | NO: 28 |
| 293T) | | GTCATGAAGG | | | | | |
| | igRNA1 | Mismatch 1: | 84.86 ± 0.45 | 0.30 ± 0.02 | 11.08 ± 0.28 | 3.75 ± 0.24 | SEQ ID |
| | | GAtGAGCAGGG | | | | | NO: 29 |
| | | TCATGAAGG | | | | | |
| | igRNA2 | Deletion: | 88.64 ± 0.40 | 0.34 ± 0.14 | 7.84 ± 0.27 | 3.17 ± 0.55 | SEQ ID |
| | | GAGAGCAGGG | | | | | NO: 30 |
| | | TCATGAAGG | | | | | |
| | igRNA3 | Insertion: | 90.08 ± 0.13 | 0.25 ± 0.08 | 7.46 ± 0.17 | 2.21 ± 0.09 | SEQ ID |
| | | GcAAGAGCAGG | | | | | NO: 31 |
| | | GTCATGAAGG | | | | | |
| | | | A6 | A8 | A6 & A8 | Others | |
| EMX1- | gRNA | Control: | 48.20 ± 0.31 | 0.90 ± 0.01 | 25.92 ± 0.38 | 24.98 ± 0.30 | SEQ ID |
| SITE3 | | GGAGCACACAT | | | | | NO: 32 |
| (HEK | | GCCCAGGTG | | | | | |
| 293T) | igRNA1 | Mismatch 1: | 72.37 ± 0.40 | 3.18 ± 0.16 | 10.62 ± 0.13 | 13.82 ± 0.24 | SEQ ID |
| | | GGtGCACACAT | | | | | NO: 33 |
| | | GCCCAGGTG | | | | | |
| | igRNA2 | Mismatch 2: | 70.71 ± 0.27 | 7.31 ± 0.27 | 9.06 ± 0.35 | 12.93 ± 0.15 | SEQ ID |
| | | GGAcCACACAT | | | | | NO: 34 |
| | | GCCCAGGTG | | | | | |
| | igRNA3 | Deletion: | 90.75 ± 0.97 | 2.18 ± 0.49 | 3.56 ± 0.22 | 3.52 ± 0.41 | SEQ ID |
| | | GAGCACACATG | | | | | NO: 35 |
| | | CCCAGGTG | | | | | |
| | igRNA4 | Insertion: | 84.44 ± 1.85 | 4.85 ± 0.44 | 6.16 ± 2.02 | 4.54 ± 0.34 | SEQ ID |
| | | GGtAGCACACA | | | | | NO: 36 |
| | | TGCCCAGGTG | | | | | |
| | | | A5 | A7 | A5 & A7 | Others | |
| VISTA | gRNA | Control: | 61.85 ± 0.55 | 0.08 ± 0.02 | 31.32 ± 0.42 | 6.74 ± 0.18 | SEQ ID |
| hs267 | | GAACACAAAG | | | | | NO: 37 |
| (HEK | | CATAGACTGC | | | | | |
| 293T) | igRNA1 | Mismatch 1: | 78.14 ± 0.23 | 0.08 ± 0.00 | 16.60 ± 0.10 | 5.18 ± 0.14 | SEQ ID |
| | | GtACACAAAGC | | | | | NO: 38 |
| | | ATAGACTGC | | | | | |
| | igRNA2 | Mismatch 2: | 87.04 ± 0.41 | 0.12 ± 0.01 | 6.55 ± 0.30 | 6.28 ± 0.13 | SEQ ID |
| | | GAAgACAAAGC | | | | | NO: 39 |
| | | ATAGACTGC | | | | | |
| | igRNA3 | Deletion: | 88.70 ± 0.03 | 0.03 ± 0.01 | 6.36 ± 0.36 | 4.92 ± 0.33 | SEQ ID |
| | | GAAACAAAGC | | | | | NO: 40 |
| | | ATAGACTGC | | | | | |
| | igRNA4 | Insertion: | 90.92 ± 0.34 | 0.06 ± 0.01 | 4.49 ± 0.45 | 4.53 ± 0.42 | SEQ ID |
| | | GAtACACAAAG | | | | | NO: 41 |
| | | CATAGACTGC | | | | | |
| | | | A5 | A7 | A5 & A7 | Others | |
| SNCA | gRNA | Control: | 79.73 ± 0.41 | 0.19 ± 0.01 | 14.54 ± 0.33 | 5.54 ± 0.21 | SEQ ID |
| (HEK | | GAACACAATGC | | | | | NO: 42 |
| 293T) | | ATAGATTGC | | | | | |
| | igRNA1 | Mismatch 1: | 82.63 ± 0.37 | 0.45 ± 0.07 | 10.50 ± 0.13 | 6.43 ± 0.24 | SEQ ID |
| | | GAtCACAATGC | | | | | NO: 43 |
| | | ATAGATTGC | | | | | |
| | igRNA2 | Mismatch 2: | 80.78 ± 0.03 | 0.59 ± 0.03 | 10.88 ± 0.18 | 7.75 ± 0.21 | SEQ ID |
| | | GAACtCAATGC | | | | | NO: 44 |
| | | ATAGATTGC | | | | | |
| | igRNA3 | Deletion: | 90.82 ± 0.62 | 0.51 ± 0.10 | 4.87 ± 0.19 | 3.80 ± 0.40 | SEQ ID |
| | | GACACAATGCA | | | | | NO: 45 |
| | | TAGATTGC | | | | | |
| | igRNA4 | Insertion: | 91.85 ± 0.50 | 0.34 ± 0.08 | 3.76 ± 0.81 | 4.05 ± 0.45 | SEQ ID |
| | | GAcACACAATG | | | | | NO: 46 |
| | | CATAGATTGC | | | | | |
| | | | A5 | A7 | A5 & A7 | Others | |
| ANO5 | gRNA | Control: | 10.26 ± 0.09 | 27.75 ± 0.03 | 48.56 ± 0.23 | 13.43 ± 0.33 | SEQ ID |
| (HEK | | TCACACACTTG | | | | | NO: 47 |
| 293T) | | ATCACAGAG | | | | | |
| | igRNA1 | Mismatch 1: | 5.64 ± 0.33 | 84.23 ± 0.98 | 3.94 ± 0.47 | 6.19 ± 0.54 | SEQ ID |
| | | TgACACACTTG | | | | | NO: 48 |
| | | ATCACAGAG | | | | | |
| | igRNA2 | Mismatch 2: | 9.81 ± 0.33 | 69.18 ± 1.12 | 9.63 ± 0.69 | 11.37 ± 0.44 | SEQ ID |
| | | TCAtACACTTGA | | | | | NO: 49 |
| | | TCACAGAG | | | | | |
| | igRNA3 | Deletion: | 8.20 ± 1.17 | 82.13 ± 0.26 | 3.99 ± 0.69 | 5.68 ± 0.83 | SEQ ID |
| | | TACACACTTGA | | | | | NO: 50 |
| | | TCACAGAG | | | | | |
| | igRNA4 | Insertion: | 3.24 ± 0.15 | 91.34 ± 0.94 | 1.86 ± 0.33 | 3.57 ± 0.49 | SEQ ID |
| | | TCaACACACTT | | | | | NO: 51 |
| | | GATCACAGAG | | | | | |
| | | | A5 | A6 | A5 & A6 | Others | |
| KCNQ2 | gRNA | Control: | 1.39 ± 0.18 | 0.22 ± 0.03 | 82.36 ± 0.49 | 16.03 ± 0.51 | SEQ ID |
| (HEK | | GAAGAAGGAG | | | | | NO: 52 |
| 293T) | | ACACCGATGA | | | | | |
| | igRNA1 | Mismatch: | 15.5 ± 0.54 | 1.25 ± 0.04 | 75.22 ± 0.39 | 8.03 ± 0.22 | SEQ ID |
| | | GAcGAAGGAGA | | | | | NO: 53 |
| | | CACCGATGA | | | | | |
| | igRNA2 | Deletion: | 27.40 ± 0.48 | 0.97 ± 0.03 | 64.75 ± 0.50 | 6.88 ± 0.34 | SEQ ID |
| | | GAGAAGGAGA | | | | | NO: 54 |
| | | CACCGATGA | | | | | |
| | igRNA3 | Insertion: | 18.15 ± 0.67 | 1.35 ± 0.33 | 70.55 ± 0.61 | 9.95 ± 0.26 | SEQ ID |
| | | GcAAGAAGGAG | | | | | NO: 55 |
| | | ACACCGATGA | | | | | |
| | | | A5 | A7 | A5 & A7 | Others | |
| NOTCH2 | gRNA | Control: | 25.36 ± 0.72 | 4.95 ± 0.14 | 61.18 ± 0.86 | 8.51 ± 0.06 | SEQ ID |
| (HEK | | TGACACAGGAG | | | | | NO: 56 |
| 293T) | | ACCTGTCAC | | | | | |
| | igRNA1 | Mismatch: | 41.91 ± 0.70 | 10.58 ± 0.24 | 40.98 ± 1.00 | 6.53 ± 0.22 | SEQ ID |
| | | TGACAtAGGAG | | | | | NO: 57 |
| | | ACCTGTCAC | | | | | |
| | igRNA2 | Deletion: | 54.77 ± 1.28 | 9.27 ± 0.82 | 29.68 ± 1.06 | 6.29 ± 0.23 | SEQ ID |
| | | TGAACAGGAG | | | | | NO: 58 |
| | | ACCTGTCAC | | | | | |
| | igRNA3 | Insertion: | 34.27 ± 1.05 | 6.64 ± 0.82 | 52.01 ± 1.72 | 7.09 ± 0.10 | SEQ ID |
| | | TGaACACAGGA | | | | | NO: 59 |
| | | GACCTGTCAC | | | | | |
| | | | A5 | A6 | A5 & A6 | Others | |
| GFI1 | gRNA | Control: | 22.76 ± 0.15 | 2.70 ± 0.10 | 71.38 ± 0.39 | 3.16 ± 0.22 | SEQ ID |
| (HEK | | TGGGAAGGGTT | | | | | NO: 60 |
| 293T) | | TCCAGAGGA | | | | | |
| | igRNA1 | Mismatch: | 48.25 ± 0.81 | 10.86 ± 0.42 | 37.42 ± 0.35 | 3.47 ± 0.38 | SEQ ID |
| | | TaGGAAGGGTT | | | | | NO: 61 |
| | | TCCAGAGGA | | | | | |
| | igRNA2 | Deletion: | 61.13 ± 2.35 | 8.13 ± 0.96 | 27.66 ± 2.71 | 3.07 ± 0.41 | SEQ ID |
| | | TGGAAGGGTTT | | | | | NO: 62 |
| | | CCAGAGGA | | | | | |
| | igRNA3 | Insertion: | 63.77 ± 1.10 | 7.19 ± 0.88 | 27.33 ± 0.70 | 1.72 ± 0.26 | SEQ ID |
| | | TGtGGAAGGGT | | | | | NO: 63 |
| | | TTCCAGAGGA | | | | | |
| | | | A5 | A7 | A5 & A7 | Others | |
| ANO5 | gRNA | Control: | 11.00 ± 0.23 | 31.94 ± 0.44 | 44.87 ± 0.31 | 12.19 ± 0.19 | SEQ ID |
| (Hela) | | TCACACACTTG | | | | | NO: 47 |
| | | ATCACAGAG | | | | | |
| | igRNA1 | Mismatch 1: | 8.22 ± 0.39 | 80.20 ± 2.99 | 6.18 ± 0.97 | 5.41 ± 0.62 | SEQ ID |
| | | TgACACACTTG | | | | | NO: 48 |
| | | ATCACAGAG | | | | | |
| | igRNA2 | Mismatch 2: | 11.17 ± 0.55 | 68.26 ± 1.97 | 9.31 ± 0.14 | 11.26 ± 0.46 | SEQ ID |
| | | TCAtACACTTGA | | | | | NO: 49 |
| | | TCACAGAG | | | | | |
| | igRNA3 | Deletion: | 5.26 ± 0.23 | 84.74 ± 2.63 | 4.72 ± 0.74 | 5.29 ± 0.11 | SEQ ID |
| | | TACACACTTGA | | | | | NO: 50 |
| | | TCACAGAG | | | | | |
| | igRNA4 | Insertion: | 4.40 ± 0.12 | 86.81 ± 2.11 | 4.04 ± 0.33 | 4.76 ± 0.34 | SEQ ID |
| | | TCaACACACTT | | | | | NO: 51 |
| | | GATCACAGAG | | | | | |

### Example 3 Realization of Precise Editing of Base at any Locus by PAM Expansion

In mammalian cells, base editors with expanded PAM frames were selected. By changing the PAM frames, it would be possible to realize precise editing at any site. As shown in FIG. 2, taking the ABE editor as an example, the site to be edited is A1, but there was A2 near A1; if NGG was used as the PAM frame, both A1 and A2 were edited in case of using a normal gRNA, while A2 was predominantly edited in case of using igRNA and the purpose of editing A1 alone could not be fulfilled in this situation; if the PAM was further expanded and NNN was used as PAM, appropriate N20 was selected and both A1 and A2 were also edited in case of using gRNA, while A1 was predominantly edited in case of using igRNA. Hence the combination of igRNA with the expanded PAM frame enabled precision editing of the base at any site.

Experimental process: The nCas9 in the representative adenine base editor NG-ABEmax plasmid, which was commonly used at present, was substituted with SpRYnCas9 capable of recognizing the PAM sequence as NRN or NYN (R was A/G and Y was C/T) to construct the adenine base editor SpRY-ABEmax that could recognize any PAM frame. HEK293T cells were plated in a 24-well plate at 5×10⁵ cells/well. When the cells in each well grew to 40% to 60% confluency, the SpRY-ABEmax editor plasmids were transfected with the Lipofectamine 2000 (Life, Invitrogen, 11668019) reagents into HEK293T, together with gRNA plasmids targeted to different editing sites and igRNA plasmids modified with insertions, deletions or mismatches at the amounts of 600 ng of editor plasmids and 300 ng of gRNA/igRNA plasmids, respectively. Transfection of each combination of plasmids was performed in triplicate. After 24 h of transfection, 5 μg/ml of puromycin (Merck, USA) was added to the medium. After 120 h of transfection, QuickExtract DNA Extraction Solution (Epicentre, USA) was used to extract genomic DNA. The regions of 200 bp to 300 bp across the edited sites were amplified by PCR using the Taq DNA polymerase (CWBIO, China). The PCR products were subjected to high-throughput sequencing to calculate the editing efficiency (GENEWIZ, China).

Experimental results: Two different genomic loci EMX1-SITE3 and NOTCH2 were selected. At the EMX1-SITE3 locus, if N20 was selected as 5′-GGAGCACACATGCCCAGGTG-3′, the PAM sequence was NGG, and the NG-ABEmax was used for editing, the editing specificity of A at the sixth position (A6) was 48.20±0.31%, and the editing specificity of A at the eighth position (A8) was 0.90±0.01%; after use of the igRNA, the editing specificity of A at the sixth position (A6) was 72.37±0.40%, and the editing specificity of A at the eighth position (A8) was 3.18±0.16% and the percentage of A8 editing was still less: if N20 was selected as 5′-GCACACATGCCCAGGTGTGG-3′, the PAM sequence was NAG, and the SpRY-ABEmax was used for editing, the original A at the sixth position would become A at the third position and the original A at the eighth position would become A at the fifth position: under such circumstances, the editing specificity of A at the third position was 0.91±0.09% and the editing specificity of A at the fifth position was 36.06±0.47% in case of editing by the corresponding gRNA; and after use of igRNA (with the base T inserted at the second position), the editing specificity of A at the third position was 0.67±0.32% and the editing specificity of A at the fifth position was 72.24±1.45%, so the percentage of editing of A at the fifth position (the original A at the seventh position) was remarkably increased. Similar effects were achieved at the NOTCH2 locus. The details were shown in Table 7 and Table 8.

TABLE 7
A-to-G Editing Efficiency of Expanded PAM Frame of Adenine Base Editor (%)

| | gRNAs/igRNAs | | PAM | | | | | |
|---|---|---|---|---|---|---|---|---|
| | | | | A5 | A7 | A5&A7 | Others | |
| NOTCH2 | gRNA | Control: | AGG | 13.93 ± 0.76 | 2.72 ± 0.18 | 33.64 ± 2.06 | 4.67 ± 0.23 | SEQ ID |
| (HEK | | TGACACAGGA | | | | | | NO: 56 |
| 293T) | | GACCTGTCAC | | | | | | |
| | igRNA | Mismatch: | AGG | 22.74 ± 0.69 | 5.74 ± 0.10 | 22.24 ± 1.10 | 3.54 ± 0.02 | SEQ ID |
| | | TGACAtAGGAG | | | | | | NO: 57 |
| | | ACCTGTCAC | | | | | | |
| | | | | A3 | A5 | A3&A5 | Others | |
| | gRNA | Expansion: | GGT | 18.35 ± 0.84 | 25.71 ± 1.03 | 44.86 ± 0.75 | 2.14 ± 0.32 | SEQ ID |
| | | ACACAGGAGA | | | | | | NO: 63 |
| | | CCTGTCACAG | | | | | | |
| | igRNA1 | Expansion + | GGT | 0.52 ± 0.09 | 67.41 ± 1.32 | 0.78 ± 0.12 | 0.09 ± 0.03 | SEQ ID |
| | | Mismatch: | | | | | | NO: 64 |
| | | AtACAGGAGAC | | | | | | |
| | | CTGTCACAG | | | | | | |
| | igRNA2 | Expansion + | GGT | 0.74 ± 0.07 | 53.42 ± 0.46 | 3.37 ± 0.19 | 0.75 ± 0.11 | SEQ ID |
| | | Deletion: | | | | | | NO: 65 |
| | | AACAGGAGAC | | | | | | |
| | | CTGTCACAG | | | | | | |
| | igRNA3 | Expansion + | GGT | 3.32 ± 0.51 | 46.88 ± 2.31 | 6.24 ± 0.63 | 1.01 ± 0.05 | SEQ ID |
| | | Insertion: | | | | | | NO: 66 |
| | | ACgACAGGAG | | | | | | |
| | | ACCTGTCACA | | | | | | |
| | | G | | | | | | |
| | | | | A6 | A8 | A6&A8 | Others | |
| EMX1- | gRNA | Control: | TGG | 19.54 ± 0.44 | 0.37 ± 0.00 | 10.51 ± 0.23 | 10.12 ± 0.11 | SEQ ID |
| SITE3 | | GGAGCACACA | | | | | | NO: 32 |
| | | TGCCCAGGTG | | | | | | |
| | igRNA1 | Mismatch: | TGG | 27.15 ± 0.29 | 1.19 ± 0.06 | 3.99 ± 0.07 | 5.19 ± 0.06 | SEQ ID |
| | | GGtGCACACAT | | | | | | NO: 33 |
| | | GCCCAGGTG | | | | | | |
| | | | | A3 | A5 | A3&A5 | Others | |
| | gRNA | Expansion: | AAG | 0.64 ± 0.01 | 25.39 ± 1.36 | 0.12 ± 0.01 | 44.26 ± 0.93 | SEQ ID |
| | | GCACACATGC | | | | | | NO: 67 |
| | | CCAGGTGTGG | | | | | | |
| | igRNA1 | Expansion + | AAG | 0.52 ± 0.03 | 43.14 ± 0.19 | 0.02 ± 0.01 | 21.34 ± 0.16 | SEQ ID |
| | | Mismatch: | | | | | | NO: 68 |
| | | GgACACATGCC | | | | | | |
| | | CAGGTGTGG | | | | | | |
| | igRNA2 | Expansion + | AAG | 0.57 ± 0.01 | 37.22 ± 0.46 | 0.03 ± 0.02 | 22.99 ± 1.30 | SEQ ID |
| | | Deletion: | | | | | | NO: 69 |
| | | GCCACATGCCC | | | | | | |
| | | AGGTGTGG | | | | | | |
| | igRNA3 | Expansion + | AAG | 0.43 ± 0.02 | 46.32 ± 0.49 | 0.01 ± 0.01 | 17.36 ± 0.25 | SEQ ID |
| | | Insertion: | | | | | | NO: 70 |
| | | GtCACACATGC | | | | | | |
| | | CCAGGTGTGG | | | | | | |

TABLE 8
A-to-G Editing Specificity of Expanded PAM Frame of Adenine Base Editor (%)

| | gRNAs/igRNAs | | PAM | | | | | |
|---|---|---|---|---|---|---|---|---|
| | | | | A5 | A7 | A5&A7 | Others | |
| NOTCH2 | gRNA | Control: | AGG | 25.36 ± 0.72 | 4.95 ± 0.14 | 61.18 ± 0.86 | 8.51 ± 0.06 | SEQ ID |
| (HEK | | TGACACAGGAG | | | | | | NO: 56 |
| 293T) | | ACCTGTCAC | | | | | | |
| | igRNA | Mismatch: | AGG | 41.91 ± 0.70 | 10.58 ± 0.24 | 40.98 ± 1.00 | 6.53 ± 0.22 | SEQ ID |
| | | TGACAtAGGAG | | | | | | NO: 57 |
| | | ACCTGTCAC | | | | | | |
| | | | | A3 | A5 | A3&A5 | Others | |
| | gRNA | Expansion: | GGT | 20.15 ± 1.32 | 28.23 ± 0.47 | 49.26 ± 0.36 | 2.35 ± 0.14 | SEQ ID |
| | | ACACAGGAGA | | | | | | NO: 63 |
| | | CCTGTCACAG | | | | | | |
| | igRNA1 | Expansion + | GGT | 0.76 ± 0.11 | 97.98 ± 1.89 | 1.13 ± 0.26 | 0.13 ± 0.31 | SEQ ID |
| | | Mismatch: | | | | | | NO: 64 |
| | | AtACAGGAGAC | | | | | | |
| | | CTGTCACAG | | | | | | |
| | igRNA2 | Expansion + | GGT | 1.27 ± 0.34 | 91.66 ± 2.57 | 5.78 ± 0.89 | 1.29 ± 0.44 | SEQ ID |
| | | Deletion: | | | | | | NO: 65 |
| | | AACAGGAGAC | | | | | | |
| | | CTGTCACAG | | | | | | |
| | igRNA3 | Expansion + | GGT | 5.78 ± 0.20 | 81.60 ± 1.69 | 10.86 ± 0.73 | 1.76 ± 0.06 | SEQ ID |
| | | Insertion: | | | | | | NO: 66 |
| | | ACgACAGGAGA | | | | | | |
| | | CCTGTCACAG | | | | | | |
| | | | | A6 | A8 | A6&A8 | Others | |
| EMX1- | gRNA | Control: | TGG | 48.20 ± 0.31 | 0.90 ± 0.01 | 25.92 ± 0.38 | 24.98-0.30 | SEQ ID |
| SITE3 | | GGAGCACACAT | | | | | | NO: 32 |
| | | GCCCAGGTG | | | | | | |
| | igRNA | Mismatch: | TGG | 72.37 ± 0.40 | 3.18 ± 0.16 | 10.62 ± 0.13 | 13.82 ± 0.24 | SEQ ID |
| | | GGtGCACACAT | | | | | | NO: 33 |
| | | GCCCAGGTG | | | | | | |
| | | | | A3 | A5 | A3&A5 | Others | |
| | gRNA | Expansion: | AAG | 0.91 ± 0.09 | 36.06 ± 0.47 | 0.17 ± 0.33 | 62.86 ± 1.29 | SEQ ID |
| | | GCACACATGCC | | | | | | NO: 67 |
| | | CAGGTGTGG | | | | | | |
| | igRNA1 | Expansion + | AAG | 0.80 ± 0.28 | 66.35 ± 0.98 | 0.03 ± 0.02 | 32.82 ± 1.09 | SEQ ID |
| | | Mismatch: | | | | | | NO: 68 |
| | | GgACACATGCC | | | | | | |
| | | CAGGTGTGG | | | | | | |
| | igRNA2 | Expansion + | AAG | 0.94 ± 0.02 | 61.21 ± 2.03 | 0.05 ± 0.01 | 37.84 ± 0.86 | SEQ ID |
| | | Deletion: | | | | | | NO: 69 |
| | | GCCACATGCCC | | | | | | |
| | | AGGTGTGG | | | | | | |
| | igRNA3 | Expansion + | AAG | 0.67 ± 0.32 | 72.24 ± 1.45 | 0.02 ± 0.00 | 27.07 ± 1.34 | SEQ ID |
| | | Insertion: | | | | | | NO: 70 |
| | | GtCACACATGCC | | | | | | |
| | | CAGGTGTGG | | | | | | |

### Example 4 Editing by SaCas9 ABE with Modified gRNA

In mammalian cells, different editing sites were edited by SaCas9 adenine base editors using modified gRNAs, and the percentages of base editing of single-window were all somewhat increased compared to the case of using unmodified gRNAs.

Experimental process: HEK293T cells were plated in a 24-well plate at 5×10⁵ cells/well. When the cells in each well grew to 40% to 60% confluency, the adenine base editor SaABEmax plasmid, which was commonly used at present, was transfected with the Lipofectamine 2000 (Life, Invitrogen, 11668019) reagent into HEK293T, together with gRNA plasmids targeted to different editing sites and igRNA plasmids modified with insertions, deletions or mismatches at the amounts of 600 ng of editor plasmids and 300 ng of gRNA/igRNA plasmids, respectively. Transfection of each combination of plasmids was performed in triplicate. After 24 h of transfection, 5 μg/ml of puromycin (Merck, USA) was added to the medium. After 120 h of transfection, QuickExtract DNA Extraction Solution (Epicentre, USA) was used to extract genomic DNA. The regions of 200 bp to 300 bp across the edited sites were amplified by PCR using the Taq DNA polymerase (CWBIO, China). The PCR products were subjected to high-throughput sequencing to calculate the editing efficiency (GENEWIZ, China).

Experimental results: Two different genomic loci CFAP61 and Query_55451 were selected. At each locus, gRNA perfectly matching the editing sites and 3 to 5 igRNAs with insertions, deletions, mismatches or the like compared to the editing sites were selected for editing.

The results showed that at the CFAP61 locus of HEK293T, when SaABEmax and igRNA (with the base G inserted at the third position) were used, the A-to-G editing specificity of single A at the eleventh position (All) at the editing sites was improved from 32.88±0.35% to 54.05±0.46%, and the editing efficiency was increased from 12.83±0.27% to 15.65±0.46%. In case of using different igRNAs, the editing specificities and editing efficiencies of single A at A9 of Query_55451 were improved accordingly. The details were shown in Table 9 and Table 10.

TABLE 9
A-to-G Editing Efficiency of Adenine Editor SaABEmax (%)

| | | | | | | SEQ ID |
|---|---|---|---|---|---|---|
| Gene | gRNAs | Mean ± SD | | | | NO: |
| | | A9 | A11 | A9&A11 | Others | |
| CFAP61 | Control: | 6.77 ± 0.13 | 12.83 ± 0.27 | 10.87 ± 0.86 | 8.55 ± 0.16 | SEQ ID |
| | GGAGAGAAAGAGA | | | | | NO: 71 |
| | AGTTGATTG | | | | | |
| | Mismatch 1: | 1.96 ± 0.17 | 11.32 ± 0.12 | 5.40 ± 0.47 | 4.74 ± 0.27 | SEQ ID |
| | GGcGAGAAAGAGA | | | | | NO: 72 |
| | AGTTGATTG | | | | | |
| | Mismatch 2: | 3.72 ± 0.34 | 14.29 ± 0.22 | 4.10 ± 0.21 | 5.07 ± 0.26 | SEQ ID |
| | GttGAGAAAGAGAA | | | | | NO: 73 |
| | GTTGATTG | | | | | |
| | Deletion: | 5.09 ± 0.24 | 12.17 ± 0.66 | 7.52 ± 0.26 | 8.02 ± 1.17 | SEQ ID |
| | GGAGAAAGAGAAG | | | | | NO: 74 |
| | TTGATTG | | | | | |
| | Insertion: | 3.31 ± 0.18 | 15.65 ± 0.46 | 5.69 ± 0.33 | 4.32 ± 0.18 | SEQ ID |
| | GGgAGAGAAAGAG | | | | | NO: 75 |
| | AAGTTGATTG | | | | | |
| | | A9 | A11 | A9&A11 | Others | |
| Query_ | Control: | 29.68 ± 0.98 | 14.43 ± 0.68 | 11.57 ± 0.60 | 12.56 ± 0.98 | SEQ ID |
| 55451 | GCTGTTGCATGAGG | | | | | NO: 76 |
| | AAAGGGAC | | | | | |
| | Mismatch 1: | 29.50 ± 0.50 | 6.40 ± 0.28 | 4.30 ± 0.66 | 5.29 ± 0.46 | SEQ ID |
| | GtaGTTGCATGAGG | | | | | NO: 77 |
| | AAAGGGAC | | | | | |
| | Mismatch 2: | 31.71 ± 0.44 | 5.24 ± 0.10 | 4.44 ± 0.20 | 4.28 ± 0.06 | SEQ ID |
| | GgTGTTGCATGAGG | | | | | NO: 78 |
| | AAAGGGAC | | | | | |
| | Deletion: | 27.77 ± 0.33 | 7.74 ± 0.46 | 5.08 ± 0.22 | 5.33 ± 0.30 | SEQ ID |
| | GCTTGCATGAGGA | | | | | NO: 79 |
| | AAGGGAC | | | | | |
| | Insertion: | 25.99 ± 0.55 | 8.32 ± 0.74 | 6.35 ± 0.31 | 5.70 ± 0.26 | SEQ ID |
| | GCtaTGTTGCATGAG | | | | | NO: 80 |
| | GAAAGGGAC | | | | | |

TABLE 10
A-to-G Editing Specificity of Adenine Editor SaABEmax (%)

| | | | | | | SEQ ID |
|---|---|---|---|---|---|---|
| Genes | gRNAs | Mean ± SD | | | | NO: |
| | | A9 | A11 | A9&A11 | Others | |
| CFAP61 | Control: | 17.36 ± 0.22 | 32.88 ± 0.35 | 27.82 ± 1.44 | 21.94 ± 1.00 | SEQ ID |
| | GGAGAGAAAGAGAA | | | | | NO: 71 |
| | GTTGATTG | | | | | |
| | Mismatch 1: | 8.37 ± 0.67 | 48.37 ± 0.84 | 23.03 ± 1.38 | 20.24 ± 1.29 | SEQ ID |
| | GGcGAGAAAGAGAA | | | | | NO: 72 |
| | GTTGATTG | | | | | |
| | Mismatch 2: | 13.66 ± 1.13 | 52.58 ± 0.90 | 15.10 ± 0.67 | 18.66 ± 0.98 | SEQ ID |
| | GttGAGAAAGAGAAG | | | | | NO: 73 |
| | TTGATTG | | | | | |
| | Deletion: | 15.54 ± 0.93 | 37.14 ± 2.60 | 22.91 ± 0.61 | 24.40 ± 3.18 | SEQ ID |
| | GGAGAAAGAGAAGT | | | | | NO: 74 |
| | TGATTG | | | | | |
| | Insertion: | 11.42 ± 0.20 | 54.05 ± 0.46 | 19.63 ± 0.53 | 14.90 ± 0.27 | SEQ ID |
| | GGgAGAGAAAGAGA | | | | | NO: 75 |
| | AGTTGATTG | | | | | |
| | | A9 | A12 | A9 & A12 | Others | |
| Query_ | Control: | 43.52 ± 1.87 | 21.15 ± 0.81 | 16.95 ± 0.50 | 18.38 ± 1.14 | SEQ ID |
| 55451 | GCTGTTGCATGAGG | | | | | NO: 76 |
| | AAAGGGAC | | | | | |
| | Mismatch 1: | 64.85 ± 0.86 | 14.07 ± 0.45 | 9.44 ± 1.38 | 11.64 ± 1.00 | SEQ ID |
| | GtaGTTGCATGAGGA | | | | | NO: 77 |
| | AAGGGAC | | | | | |
| | Mismatch 2: | 69.42 ± 0.51 | 11.48 ± 0.20 | 9.72 ± 0.34 | 9.38 ± 0.23 | SEQ ID |
| | GgTGTTGCATGAGGA | | | | | NO: 78 |
| | AAGGGAC | | | | | |
| | Deletion: | 60.48 ± 0.71 | 16.86 ± 0.96 | 11.07 ± 0.55 | 11.60 ± 0.60 | SEQ ID |
| | GCTTGCATGAGGAA | | | | | NO: 79 |
| | AGGGAC | | | | | |
| | Insertion: | 56.08 ± 1.63 | 17.94 ± 1.39 | 13.69 ± 0.47 | 12.29 ± 0.66 | SEQ ID |
| | GCtaTGTTGCATGAG | | | | | NO: 80 |
| | GAAAGGGAC | | | | | |

SEQ ID NO: 81:

| | |
|---|---|
| gttttagagc tagaaatagc aagttaaaat aaggctagtc cgttatcaac ttgaaaaagt | 60 |
| ggcaccgagt cggtgc | 76 |

SEQ ID NO: 82:

| | |
|---|---|
| gttttagtac tctggaaaca gaatctacta aaacaaggca aaatgccgtg tttatctcgt | 60 |
| caacttgttg gcgaga | 76 |

SEQ ID NO: 83:

| | |
|---|---|
| SEVEFSHEYW MRHALTLAKR ARDEREVPVG AVLVLNNRVI GEGWNRAIGL HDPTAHAEIM | 60 |
| ALRQGGLVMQ NYRLIDATLY VTFEPCVMCA GAMIHSRIGR VVFGVRNAKT GAAGSLMDVL | 120 |
| HYPGMNHRVE ITEGILADEC AALLCYFFRM PRQVFNAQKK AQSSTD | 166 |

SEQ ID NO: 84:

| | |
|---|---|
| SEVEFSHEYW MRHALTLAKR ARDEREVPVG AVLVLNNRVI GEGWNRAIGL HDPTAHAEIM | 60 |
| ALRQGGLVMQ NYRLIDATLY VTFEPCVMCA GAMIHSRIGR VVFGVRNSKR GAAGSLMNVL | 120 |
| NYPGMNHRVE ITEGILADEC AALLCDFYRM PRQVFNAQKK AQSSIN | 166 |

SEQ ID NO: 85:

| | |
|---|---|
| MDKKYSIGLA IGTNSVGWAV ITDEYKVPSK KFKVLGNTDR HSIKKNLIGA LLFDSGETAE | 60 |
| ATRLKRTARR RYTRRKNRIC YLQEIFSNEM AKVDDSFFHR LEESFLVEED KKHERHPIFG | 120 |
| NIVDEVAYHE KYPTIYHLRK KLVDSTDKAD LRLIYLALAH MIKFRGHFLI EGDLNPDNSD | 180 |
| VDKLFIQLVQ TYNQLFEENP INASGVDAKA ILSARLSKSR RLENLIAQLP GEKKNGLFGN | 240 |
| LIALSLGLTP NFKSNFDLAE DAKLQLSKDT YDDDLDNLLA QIGDQYADLF LAAKNLSDAI | 300 |
| LLSDILRVNT EITKAPLSAS MIKRYDEHHQ DLTLLKALVR QQLPEKYKEI FFDQSKNGYA | 360 |
| GYIDGGASQE EFYKFIKPIL EKMDGTEELL VKLNREDLLR KQRTFDNGSI PHQIHLGELH | 420 |
| AILRRQEDFY PFLKDNREKI EKILTFRIPY YVGPLARGNS RFAWMTRKSE ETITPWNFEE | 480 |
| VVDKGASAQS FIERMTNFDK NLPNEKVLPK HSLLYEYFTV YNELTKVKYV TEGMRKPAFL | 540 |
| SGEQKKAIVD LLFKTNRKVT VKQLKEDYFK KIECFDSVEI SGVEDRFNAS LGTYHDLLKI | 600 |
| IKDKDFLDNE ENEDILEDIV LTLTLFEDRE MIEERLKTYA HLFDDKVMKQ LKRRRYTGWG | 660 |
| RLSRKLINGI RDKQSGKTIL DFLKSDGFAN RNFMQLIHDD SLTFKEDIQK AQVSGQGDSL | 720 |
| HEHIANLAGS PAIKKGILQT VKVVDELVKV MGRHKPENIV IEMARENQTT QKGQKNSRER | 780 |
| MKRIEEGIKE LGSQILKEHP VENTQLQNEK LYLYYLQNGR DMYVDQELDI NRLSDYDVDA | 840 |
| IVPQSFLKDD SIDNKVLTRS DKNRGKSDNV PSEEVVKKMK NYWRQLLNAK LITQRKFDNL | 900 |
| TKAERGGLSE LDKAGFIKRQ LVETRQITKH VAQILDSRMN TKYDENDKLI REVKVITLKS | 960 |
| KLVSDFRKDF QFYKVREINN YHHAHDAYLN AVVGTALIKK YPKLESEFVY GDYKVYDVRK | 1020 |
| MIAKSEQEIG KATAKYFFYS NIMNFFKTEI TLANGEIRKR PLIETNGETG EIVWDKGRDF | 1080 |
| ATVRKVLSMP QVNIVKKTEV QTGGFSKESI LPKRNSDKLI ARKKDWDPKK YGGFDSPTVA | 1140 |
| YSVLVVAKVE KGKSKKLKSV KELLGITIME RSSFEKNPID FLEAKGYKEV KKDLIIKLPK | 1200 |
| YSLFELENGR KRMLASAGEL QKGNELALPS KYVNFLYLAS HYEKLKGSPE DNEQKQLFVE | 1260 |
| QHKHYLDEII EQISEFSKRV ILADANLDKV LSAYNKHRDK PIREQAENII HLFTLTNLGA | 1320 |
| PAAFKYFDTT IDRKRYTSTK EVLDATLIHQ SITGLYETRI DLSQLGGD | 1368 |

SEQ ID NO: 86:

| | |
|---|---|
| MDKKYSIGLA IGTNSVGWAV ITDEYKVPSK KFKVLGNTDR HSIKKNLIGA LLFDSGETAE | 60 |
| ATRLKRTARR RYTRRKNRIC YLQEIFSNEM AKVDDSFFHR LEESFLVEED KKHERHPIFG | 120 |
| NIVDEVAYHE KYPTIYHLRK KLVDSTDKAD LRLIYLALAH MIKFRGHFLI EGDLNPDNSD | 180 |
| VDKLFIQLVQ TYNQLFEENP INASGVDAKA ILSARLSKSR RLENLIAQLP GEKKNGLFGN | 240 |
| LIALSLGLTP NFKSNFDLAE DAKLQLSKDT YDDDLDNLLA QIGDQYADLF LAAKNLSDAI | 300 |
| LLSDILRVNT EITKAPLSAS MIKRYDEHHQ DLTLLKALVR QQLPEKYKEI FFDQSKNGYA | 360 |
| GYIDGGASQE EFYKFIKPIL EKMDGTEELL VKLNREDLLR KQRTFDNGSI PHQIHLGELH | 420 |
| AILRRQEDFY PFLKDNREKI EKILTFRIPY YVGPLARGNS RFAWMTRKSE ETITPWNFEE | 480 |
| VVDKGASAQS FIERMTNFDK NLPNEKVLPK HSLLYEYFTV YNELTKVKYV TEGMRKPAFL | 540 |
| SGEQKKAIVD LLFKTNRKVT VKQLKEDYFK KIECFDSVEI SGVEDRFNAS LGTYHDLLKI | 600 |
| IKDKDFLDNE ENEDILEDIV LTLTLFEDRE MIEERLKTYA HLFDDKVMKQ LKRRRYTGWG | 660 |
| RLSRKLINGI RDKQSGKTIL DFLKSDGFAN RNFMQLIHDD SLTFKEDIQK AQVSGQGDSL | 720 |
| HEHIANLAGS PAIKKGILQT VKVVDELVKV MGRHKPENIV IEMARENQTT QKGQKNSRER | 780 |
| MKRIEEGIKE LGSQILKEHP VENTQLQNEK LYLYYLQNGR DMYVDQELDI NRLSDYDVDH | 840 |
| IVPQSFLKDD SIDNKVLTRS DKNRGKSDNV PSEEVVKKMK NYWRQLLNAK LITQRKFDNL | 900 |
| TKAERGGLSE LDKAGFIKRQ LVETRQITKH VAQILDSRMN TKYDENDKLI REVKVITLKS | 960 |
| KLVSDFRKDF QFYKVREINN YHHAHDAYLN AVVGTALIKK YPKLESEFVY GDYKVYDVRK | 1020 |
| MIAKSEQEIG KATAKYFFYS NIMNFFKTEI TLANGEIRKR PLIETNGETG EIVWDKGRDF | 1080 |
| ATVRKVLSMP QVNIVKKTEV QTGGFSKESI LPKRNSDKLI ARKKDWDPKK YGGFDSPTVA | 1140 |
| YSVLVVAKVE KGKSKKLKSV KELLGITIME RSSFEKNPID FLEAKGYKEV KKDLIIKLPK | 1200 |
| YSLFELENGR KRMLASAGEL QKGNELALPS KYVNFLYLAS HYEKLKGSPE DNEQKQLFVE | 1260 |
| QHKHYLDEII EQISEFSKRV ILADANLDKV LSAYNKHRDK PIREQAENII HLFTLTNLGA | 1320 |
| PAAFKYFDTT IDRKRYTSTK EVLDATLIHQ SITGLYETRI DLSQLGGD | 1368 |

SEQ ID NO: 87:

| | |
|---|---|
| MKRNYILGLA IGITSVGYGI IDYETRDVID AGVRLFKEAN VENNEGRRSK RGARRLKRRR | 60 |
| RHRIQRVKKL LFDYNLLTDH SELSGINPYE ARVKGLSQKL SEEEFSAALL HLAKRRGVHN | 120 |
| VNEVEEDTGN ELSTKEQISR NSKALEEKYV AELQLERLKK DGEVRGSINR FKTSDYVKEA | 180 |
| KQLLKVQKAY HQLDQSFIDT YIDLLETRRT YYEGPGEGSP FGWKDIKEWY EMLMGHCTYF | 240 |
| PEELRSVKYA YNADLYNALN DLNNLVITRD ENEKLEYYEK FQIIENVFKQ KKKPTLKQIA | 300 |
| KEILVNEEDI KGYRVTSTGK PEFTNLKVYH DIKDITARKE IIENAELLDQ IAKILTIYQS | 360 |
| SEDIQEELTN LNSELTQEEI EQISNLKGYT GTHNLSLKAI NLILDELWHT NDNQIAIFNR | 420 |
| LKLVPKKVDL SQQKEIPTTL VDDFILSPVV KRSFIQSIKV INAIIKKYGL PNDIIIELAR | 480 |
| EKNSKDAQKM INEMQKRNRQ TNERIEEIIR TTGKENAKYL IEKIKLHDMQ EGKCLYSLEA | 540 |
| IPLEDLLNNP FNYEVDHIIP RSVSFDNSFN NKVLVKQEEA SKKGNRTPFQ YLSSSDSKIS | 600 |
| YETFKKHILN LAKGKGRISK TKKEYLLEER DINRFSVQKD FINRNLVDTR YATRGLMNLL | 660 |
| RSYFRVNNLD VKVKSINGGF TSFLRRKWKF KKERNKGYKH HAEDALIIAN ADFIFKEWKK | 720 |
| LDKAKKVMEN QMFEEKQAES MPEIETEQEY KEIFITPHQI KHIKDFKDYK YSHRVDKKPN | 780 |
| RELINDTLYS TRKDDKGNTL IVNNLNGLYD KDNDKLKKLI NKSPEKLLMY HHDPQTYQKL | 840 |
| KLIMEQYGDE KNPLYKYYEE TGNYLTKYSK KDNGPVIKKI KYYGNKLNAH LDITDDYPNS | 900 |
| RNKVVKLSLK PYRFDVYLDN GVYKFVTVKN LDVIKKENYY EVNSKCYEEA KKLKKISNQA | 960 |
| EFIASFYNND LIKINGELYR VIGVNNDLLN RIEVNMIDIT YREYLENMND KRPPRIIKTI | 1020 |
| ASKTQSIKKY STDILGNLYE VKSKKHPQII KKG | 1053 |

SEQ ID NO: 88:

| | |
|---|---|
| MKRNYILGLA IGITSVGYGI IDYETRDVID AGVRLFKEAN VENNEGRRSK RGARRLKRRR | 60 |
| RHRIQRVKKL LFDYNLLTDH SELSGINPYE ARVKGLSQKL SEEEFSAALL HLAKRRGVHN | 120 |
| VNEVEEDTGN ELSTKEQISR NSKALEEKYV AELQLERLKK DGEVRGSINR FKTSDYVKEA | 180 |
| KQLLKVQKAY HQLDQSFIDT YIDLLETRRT YYEGPGEGSP FGWKDIKEWY EMLMGHCTYF | 240 |
| PEELRSVKYA YNADLYNALN DLNNLVITRD ENEKLEYYEK FQIIENVFKQ KKKPTLKQIA | 300 |
| KEILVNEEDI KGYRVTSTGK PEFTNLKVYH DIKDITARKE IIENAELLDQ IAKILTIYQS | 360 |
| SEDIQEELTN LNSELTQEEI EQISNLKGYT GTHNLSLKAI NLILDELWHT NDNQIAIFNR | 420 |
| LKLVPKKVDL SQQKEIPTTL VDDFILSPVV KRSFIQSIKV INAIIKKYGL PNDIIIELAR | 480 |
| EKNSKDAQKM INEMQKRNRQ TNERIEEIIR TTGKENAKYL IEKIKLHDMQ EGKCLYSLEA | 540 |
| IPLEDLLNNP FNYEVDHIIP RSVSFDNSFN NKVLVKQEEN SKKGNRTPFQ YLSSSDSKIS | 600 |
| YETFKKHILN LAKGKGRISK TKKEYLLEER DINRFSVQKD FINRNLVDTR YATRGLMNLL | 660 |
| RSYFRVNNLD VKVKSINGGF TSFLRRKWKF KKERNKGYKH HAEDALIIAN ADFIFKEWKK | 720 |
| LDKAKKVMEN QMFEEKQAES MPEIETEQEY KEIFITPHQI KHIKDFKDYK YSHRVDKKPN | 780 |
| RELINDTLYS TRKDDKGNTL IVNNLNGLYD KDNDKLKKLI NKSPEKLLMY HHDPQTYQKL | 840 |
| KLIMEQYGDE KNPLYKYYEE TGNYLTKYSK KDNGPVIKKI KYYGNKLNAH LDITDDYPNS | 900 |
| RNKVVKLSLK PYRFDVYLDN GVYKFVTVKN LDVIKKENYY EVNSKCYEEA KKLKKISNQA | 960 |
| EFIASFYNND LIKINGELYR VIGVNNDLLN RIEVNMIDIT YREYLENMND KRPPRIIKTI | 1020 |
| ASKTQSIKKY STDILGNLYE VKSKKHPQII KKG | 1053 |

SEQ ID NO: 89:

| | |
|---|---|
| MSKLEKFTNC YSLSKTLRFK AIPVGKTQEN IDNKRLLVED EKRAEDYKGV KKLLDRYYLS | 60 |
| FINDVLHSIK LKNLNNYISL FRKKTRTEKE NKELENLEIN LRKEIAKAFK GNEGYKSLFK | 120 |
| KDIIETILPE FLDDKDEIAL VNSFNGFTTA FTGFFDNREN MFSEEAKSTS IAFRCINENL | 180 |
| TRYISNMDIF EKVDAIFDKH EVQEIKEKIL NSDYDVEDFF EGEFFNFVLT QEGIDVYNAI | 240 |
| IGGFVTESGE KIKGLNEYIN LYNQKTKQKL PKFKPLYKQV LSDRESLSFY GEGYTSDEEV | 300 |
| LEVFRNTLNK NSEIFSSIKK LEKLFKNFDE YSSAGIFVKN GPAISTISKD IFGEWNVIRD | 360 |
| KWNAEYDDIH LKKKAVVTEK YEDDRRKSFK KIGSFSLEQL QEYADADLSV VEKLKEIIIQ | 420 |
| KVDEIYKVYG SSEKLFDADF VLEKSLKKND AVVAIMKDLL DSVKSFENYI KAFFGEGKET | 480 |
| NRDESFYGDF VLAYDILLKV DHIYDAIRNY VTQKPYSKDK FKLYFQNPQF MGGWDKDKET | 540 |
| DYRATILRYG SKYYLAIMDK KYAKCLQKID KDDVNGNYEK INYKLLPGPN KMLPKVFFSK | 600 |
| KWMAYYNPSE DIQKIYKNGT FKKGDMFNLN DCHKLIDFFK DSISRYPKWS NAYDFNFSET | 660 |
| EKYKDIAGFY REVEEQGYKV SFESASKKEV DKLVEEGKLY MFQIYNKDFS DKSHGTPNLH | 720 |
| TMYFKLLFDE NNHGQIRLSG GAELFMRRAS LKKEELVVHP ANSPIANKNP DNPKKTTTLS | 780 |
| YDVYKDKRFS EDQYELHIPI AINKCPKNIF KINTEVRVLL KHDDNPYVIG IARGERNLLY | 840 |
| IVVVDGKGNI VEQYSLNEII NNFNGIRIKT DYHSLLDKKE KERFEARQNW TSIENIKELK | 900 |
| AGYISQVVHK ICELVEKYDA VIALADLNSG FKNSRVKVEK QVYQKFEKML IDKLNYMVDK | 960 |
| KSNPCATGGA LKGYQITNKF ESFKSMSTQN GFIFYIPAWL TSKIDPSTGF VNLLKTKYTS | 1020 |
| IADSKKFISS FDRIMYVPEE DLFEFALDYK NFSRTDADYI KKWKLYSYGN RIRIFRNPKK | 1080 |
| NNVFDWEEVC LTSAYKELFN KYGINYQQGD IRALLCEQSD KAFYSSFMAL MSLMLQMRNS | 1140 |
| ITGRTDVAFL ISPVKNSDGI FYDSRNYEAQ ENAILPKNAD ANGAYNIARK VLWAIGQFKK | 1200 |
| AEDEKLDKVK IAISNKEWLE YAQTSVKH | 1228 |

The examples of the present disclosure described above are merely instances provided to explain the present disclosure clearly, rather than limitations on the embodiments of the present disclosure. For a person skilled in the art, other variations or modifications in different forms may be further made on the basis of the above explanations. It is not necessary nor is it possible to exhaust all embodiments. Any modifications, equivalents, improvements and so on made within the spirit and principle of the present disclosure shall all be covered within the scope of protection for the claims of the present disclosure.

1. A method for constructing a gRNA mutant, wherein the method comprises:
a mutation step: mutating a guide sequence region in a gRNA that is hybridized with a target sequence of a nucleic acid of interest, such that a substitution, deletion or insertion of one or more bases occurs at one or more positions of the guide sequence region, to form a mutation sequence region containing a mutated nucleotide; and
a screening step: screening a mutant with a narrowed editing window for a base editor as compared to an unmutated gRNA, to obtain the gRNA mutant.

2. The method according to claim 1, wherein the screening step comprises:
screening the mutant with an editing window for the base editor being a single base, to obtain the gRNA mutant.

3. The method according to claim 1 or 2, wherein the guide sequence region has a first end proximal to a PAM sequence of the nucleic acid of interest and a second end distal to the PAM sequence; and
the guide sequence region has m nucleotides, and any one of the mutated nucleotides is located at the position of the n^{th} nucleotide starting from the second end, 1≤n≤m, where m and n are positive integers; preferably 1≤n≤m/2, more preferably 1≤n≤m/3.

4. The method according to claim 3, wherein m is any integer from 15 to 30, preferably any integer from 15 to 25;
optionally, any one of the mutated nucleotides is located at a position of the 1^{st} to 10^{th} nucleotides, preferably at a position of the 2^{nd} to 10^{th} nucleotides, more preferably at a position of the 2^{nd} to 7^{th} nucleotides, more preferably at a position of the 2^{nd} to 6^{th} nucleotides, starting from the second end.

5. The method according to claim 1, wherein the mutated nucleotide contains the substitution, deletion or insertion of 1 to 10 bases, preferably the substitution, deletion or insertion of 1 to 5 bases, more preferably the substitution, deletion or insertion of 1 to 3 bases.

6. A method for narrowing an editing window of a base editor, wherein the method comprises constructing a gRNA mutant by the method according to claim 1; preferably, the editing window of the base editor is one base.

7. A gRNA mutant, wherein the gRNA mutant is constructed by the method according to claim 1; preferably, the gRNA mutant is used for a base editor with an editing window being a single nucleotide site;
preferably, the gRNA mutant comprises a structure shown in either 5′-guide sequence region-repetitive sequence region-3′ or 5′-repetitive sequence region-guide sequence region-3′.

8. The gRNA mutant according to claim 7, wherein the guide sequence region has a first end proximal to a PAM sequence of the nucleic acid of interest and a second end distal to the PAM sequence; and
the guide sequence region has m nucleotides, and any one of the mutated nucleotides is located at the position of the n^{th} nucleotide starting from the second end, 1<n≤m, where m and n are positive integers; preferably 1≤n≤m/2, more preferably 1≤n≤m/3.

9. The gRNA mutant according to claim 8, wherein m is any integer from 15 to 30, preferably any integer from 15 to 25;
optionally, any one of the mutated nucleotides is located at a position of the 1^{st} to 12^{th} nucleotides, preferably at a position of the 2^{nd} to 10^{th} nucleotides, more preferably at a position of the 2^{nd} to 7^{th} nucleotides, more preferably at a position of the 2^{nd} to 6^{th} nucleotides, starting from the second end.

10. The gRNA mutant according to claim 7, wherein the mutated nucleotide contains a substitution, deletion or insertion of 1 to 10 bases, preferably a substitution, deletion or insertion of 1 to 5 bases, more preferably a substitution, deletion or insertion of 1 to 3 bases.

11. An isolated polynucleotide, wherein the isolated polynucleotide encodes the gRNA mutant according to claim 7.

12. A recombinant expression vector, wherein the recombinant expression vector contains the isolated polynucleotide according to claim 11.

13. A recombinant host cell, wherein the recombinant host cell contains the recombinant expression vector according to claim 12.

14. A base editor, wherein the base editor comprises either of the following (i) and (ii) and either of the following (iii) and (iv):
(i) the gRNA mutant according to claim 7;
(ii) a polynucleotide, recombinant expression vector or recombinant host cell that expresses the gRNA mutant according to claim 7;
(iii) a fusion protein, wherein the fusion protein contains a first domain binding to the gRNA and a second domain having a base modification activity; and
(iv) a polynucleotide, recombinant expression vector or recombinant host cell that expresses the fusion protein as shown in (iii);
preferably, the first domain is a Cas protein mutant, homologue or polypeptide fragment having a lost or reduced nuclease activity;
optionally, the first domain is at least one selected from the group consisting of: a Cas9 protein mutant, homologue or polypeptide fragment having a lost or reduced nuclease activity and a Cas12a protein mutant, homologue or polypeptide fragment having a lost or reduced nuclease activity; preferably, the first domain is SpdCas9, SpnCas9, SadCas9, SanCas9, or LbdCpf1.

15. The base editor according to claim 14, wherein the second domain is a polypeptide having a deaminase activity; optionally, the second domain is an adenine deaminase or a mutant, homologue or polypeptide fragment having or partially having an adenine deaminase activity of the adenine deaminase; optionally, the second domain is a cytosine deaminase or a mutant, homologue or polypeptide fragment having or partially having a cytosine deaminase activity of the cytosine deaminase;
optionally, the second domain is an enzyme having the adenine deaminase activity, wherein the enzyme having the adenine deaminase activity is at least one selected from the group consisting of the following (c₁) and (c₂):
(c₁) an Escherichia coli-derived adenosine deaminase, a human-derived adenosine deaminase, or a mouse-derived adenosine deaminase; and
(c₂) a mutant, homologue or polypeptide of the adenosine deaminase as shown in (c₁) that has or partially has an adenosine deaminase activity;
optionally, the second domain is an enzyme having the cytosine deaminase activity, wherein the enzyme having the cytosine deaminase activity is at least one selected from the group consisting of the following (d₁) and (d₂):
(d₁) AID, APOBEC3A, APOBEC3G, APOBEC1, or CDA1; and
(d₂) a mutant, homologue or polypeptide of an enzyme as shown in (d₁) that has or partially has the cytosine deaminase activity.

16. A composition, wherein the composition comprises the gRNA mutant according to claim 7;
optionally, the composition further comprises one or more pharmaceutically acceptable carriers.

17. (canceled)

18. A method for gene editing in a cell or subject, wherein the method comprises bringing the cell or subject into contact with any one of the gRNA mutant according to claim 7;
preferably, the gene editing is editing of a single base; more preferably, the gene editing is a substitution of one base.

19. A method for treating or preventing a disease, wherein the method comprises administering to a subject the gRNA mutant according to claim 7;
optionally, a route of the administration includes: intravenous administration, intraperitoneal administration, intracoronary administration, intra-arterial administration, intradermal administration, subcutaneous administration, transdermal delivery, intratracheal administration, intra-articular administration, intraventricular administration, inhalation, intracerebral administration, transumbilical administration, oral administration, intraocular administration, pulmonary administration, catheter injection, administration via a suppository, a viral vector, and a lipid nanomaterial, and direct injection into a tissue.

20. A method for preparing a reagent or kit for single-base editing, wherein the method comprises using the gRNA mutant according to claim 7.

21. A method for preparing a medication for gene therapy, wherein the method comprises using the gRNA mutant according to claim 7.


SEQUENCE LISTING (89 sequences)

SEQ ID NO: 1 - RNA, 15 nt, synthetic construct
  misc_feature 1..15: note = guide sequence
  source 1..15: mol_type = other RNA
gcacaccccagagcg

SEQ ID NO: 2 - RNA, 20 nt, synthetic construct
  modified_base 2..3: mod_base = OTHER; note = thymine
  modified_base 5: mod_base = OTHER; note = thymine
  modified_base 7..9: mod_base = OTHER; note = thymine
  misc_feature 1..20: note = guide sequence
  source 1..20: mol_type = other RNA
gttatctttaccccagagcg

SEQ ID NO: 3 - RNA, 20 nt, synthetic construct
  modified_base 2: mod_base = OTHER; note = thymine
  modified_base 5: mod_base = OTHER; note = thymine
  modified_base 7..9: mod_base = OTHER; note = thymine
  misc_feature 1..20: note = guide sequence
  source 1..20: mol_type = other RNA
gtcatgtttaccccagagcg

SEQ ID NO: 4 - RNA, 19 nt, synthetic construct
  modified_base 2: mod_base = OTHER; note = thymine
  modified_base 4: mod_base = OTHER; note = thymine
  modified_base 6..8: mod_base = OTHER; note = thymine
  misc_feature 1..19: note = guide sequence
  source 1..19: mol_type = other RNA
gtatctttaccccagagcg

SEQ ID NO: 5 - RNA, 22 nt, synthetic construct
  modified_base 2: mod_base = OTHER; note = thymine
  modified_base 7: mod_base = OTHER; note = thymine
  modified_base 9..11: mod_base = OTHER; note = thymine
  misc_feature 1..22: note = guide sequence
  source 1..22: mol_type = other RNA
gtgacatctttaccccagagcg

SEQ ID NO: 6 - RNA, 20 nt, synthetic construct
  modified_base 7: mod_base = OTHER; note = thymine
  modified_base 9: mod_base = OTHER; note = thymine
  modified_base 11: mod_base = OTHER; note = thymine
  modified_base 13: mod_base = OTHER; note = thymine
  modified_base 16: mod_base = OTHER; note = thymine
  misc_feature 1..20: note = guide sequence
  source 1..20: mol_type = other RNA
gacacgtctgtgtagtgcac

SEQ ID NO: 7 - RNA, 20 nt, synthetic construct
  modified_base 3: mod_base = OTHER; note = thymine
  modified_base 7: mod_base = OTHER; note = thymine
  modified_base 9: mod_base = OTHER; note = thymine
  modified_base 11: mod_base = OTHER; note = thymine
  modified_base 13: mod_base = OTHER; note = thymine
  modified_base 16: mod_base = OTHER; note = thymine
  misc_feature 1..20: note = guide sequence
  source 1..20: mol_type = other RNA
gatacgtctgtgtagtgcac

SEQ ID NO: 8 - RNA, 20 nt, synthetic construct
  modified_base 2..3: mod_base = OTHER; note = thymine
  modified_base 7: mod_base = OTHER; note = thymine
  modified_base 9: mod_base = OTHER; note = thymine
  modified_base 11: mod_base = OTHER; note = thymine
  modified_base 13: mod_base = OTHER; note = thymine
  modified_base 16: mod_base = OTHER; note = thymine
  misc_feature 1..20: note = guide sequence
  source 1..20: mol_type = other RNA
gttacgtctgtgtagtgcac

SEQ ID NO: 9 - RNA, 19 nt, synthetic construct
  modified_base 6: mod_base = OTHER; note = thymine
  modified_base 8: mod_base = OTHER; note = thymine
  modified_base 10: mod_base = OTHER; note = thymine
  modified_base 12: mod_base = OTHER; note = thymine
  modified_base 15: mod_base = OTHER; note = thymine
  misc_feature 1..19: note = guide sequence
  source 1..19: mol_type = other RNA
gcacgtctgtgtagtgcac

SEQ ID NO: 10 - RNA, 22 nt, synthetic construct
  modified_base 4: mod_base = OTHER; note = thymine
  modified_base 9: mod_base = OTHER; note = thymine
  modified_base 11: mod_base = OTHER; note = thymine
  modified_base 13: mod_base = OTHER; note = thymine
  modified_base 15: mod_base = OTHER; note = thymine
  modified_base 18: mod_base = OTHER; note = thymine
  misc_feature 1..22: note = guide sequence
  source 1..22: mol_type = other RNA
gagtcacgtctgtgtagtgcac

SEQ ID NO: 11 - RNA, 20 nt, synthetic construct
  modified_base 2: mod_base = OTHER; note = thymine
  modified_base 5: mod_base = OTHER; note = thymine
  modified_base 7..8: mod_base = OTHER; note = thymine
  modified_base 11: mod_base = OTHER; note = thymine
  modified_base 14..15: mod_base = OTHER; note = thymine
  modified_base 19: mod_base = OTHER; note = thymine
  misc_feature 1..20: note = guide sequence
  source 1..20: mol_type = other RNA
gtcatcttagtcattacctg

SEQ ID NO: 12 - RNA, 20 nt, synthetic construct
  modified_base 2..3: mod_base = OTHER; note = thymine
  modified_base 5: mod_base = OTHER; note = thymine
  modified_base 7..8: mod_base = OTHER; note = thymine
  modified_base 11: mod_base = OTHER; note = thymine
  modified_base 14..15: mod_base = OTHER; note = thymine
  modified_base 19: mod_base = OTHER; note = thymine
  misc_feature 1..20: note = guide sequence
  source 1..20: mol_type = other RNA
gttatcttagtcattacctg

SEQ ID NO: 13 - RNA, 19 nt, synthetic construct
  modified_base 4: mod_base = OTHER; note = thymine
  modified_base 6..7: mod_base = OTHER; note = thymine
  modified_base 10: mod_base = OTHER; note = thymine
  modified_base 13..14: mod_base = OTHER; note = thymine
  modified_base 18: mod_base = OTHER; note = thymine
  misc_feature 1..19: note = guide sequence
  source 1..19: mol_type = other RNA
gcatcttagtcattacctg

SEQ ID NO: 14 - RNA, 21 nt, synthetic construct
  modified_base 2: mod_base = OTHER; note = thymine
  modified_base 6: mod_base = OTHER; note = thymine
  modified_base 8..9: mod_base = OTHER; note = thymine
  modified_base 12: mod_base = OTHER; note = thymine
  modified_base 15..16: mod_base = OTHER; note = thymine
  modified_base 20: mod_base = OTHER; note = thymine
  misc_feature 1..21: note = guide sequence
  source 1..21: mol_type = other RNA
gtacatcttagtcattacctg

SEQ ID NO: 15 - RNA, 20 nt, synthetic construct
  modified_base 2: mod_base = OTHER; note = thymine
  modified_base 4: mod_base = OTHER; note = thymine
  modified_base 7..9: mod_base = OTHER; note = thymine
  modified_base 14: mod_base = OTHER; note = thymine
  modified_base 17: mod_base = OTHER; note = thymine
  misc_feature 1..20: note = guide sequence
  source 1..20: mol_type = other RNA
gtgtcctttgagctcgtgca

SEQ ID NO: 16 - RNA, 20 nt, synthetic construct
  modified_base 2: mod_base = OTHER; note = thymine
  modified_base 4: mod_base = OTHER; note = thymine
  modified_base 6..9: mod_base = OTHER; note = thymine
  modified_base 14: mod_base = OTHER; note = thymine
  modified_base 17: mod_base = OTHER; note = thymine
  misc_feature 1..20: note = guide sequence
  source 1..20: mol_type = other RNA
gtgtcttttgagctcgtgca

SEQ ID NO: 17 - RNA, 19 nt, synthetic construct
  modified_base 2..3: mod_base = OTHER; note = thymine
  modified_base 6..8: mod_base = OTHER; note = thymine
  modified_base 13: mod_base = OTHER; note = thymine
  modified_base 16: mod_base = OTHER; note = thymine
  misc_feature 1..19: note = guide sequence
  source 1..19: mol_type = other RNA
gttcctttgagctcgtgca

SEQ ID NO: 18 - RNA, 21 nt, synthetic construct
  modified_base 2..3: mod_base = OTHER; note = thymine
  modified_base 5: mod_base = OTHER; note = thymine
  modified_base 8..10: mod_base = OTHER; note = thymine
  modified_base 15: mod_base = OTHER; note = thymine
  modified_base 18: mod_base = OTHER; note = thymine
  misc_feature 1..21: note = guide sequence
  source 1..21: mol_type = other RNA
gttgtcctttgagctcgtgca

SEQ ID NO: 19 - RNA, 20 nt, synthetic construct
  modified_base 5: mod_base = OTHER; note = thymine
  modified_base 9: mod_base = OTHER; note = thymine
  modified_base 11: mod_base = OTHER; note = thymine
  modified_base 16: mod_base = OTHER; note = thymine
  modified_base 18: mod_base = OTHER; note = thymine
  misc_feature 1..20: note = guide sequence
  source 1..20: mol_type = other RNA
ggcatcagtgtgacatctgc

SEQ ID NO: 20 - RNA, 20 nt, synthetic construct
  modified_base 3: mod_base = OTHER; note = thymine
  modified_base 5: mod_base = OTHER; note = thymine
  modified_base 9: mod_base = OTHER; note = thymine
  modified_base 11: mod_base = OTHER; note = thymine
  modified_base 16: mod_base = OTHER; note = thymine
  modified_base 18: mod_base = OTHER; note = thymine
  misc_feature 1..20: note = guide sequence
  source 1..20: mol_type = other RNA
ggtatcagtgtgacatctgc

SEQ ID NO: 21 - RNA, 18 nt, synthetic construct
  modified_base 3: mod_base = OTHER; note = thymine
  modified_base 7: mod_base = OTHER; note = thymine
  modified_base 9: mod_base = OTHER; note = thymine
  modified_base 14: mod_base = OTHER; note = thymine
  modified_base 16: mod_base = OTHER; note = thymine
  misc_feature 1..18: note = guide sequence
  source 1..18: mol_type = other RNA
gatcagtgtgacatctgc

SEQ ID NO: 22 - RNA, 21 nt, synthetic construct
  modified_base 2: mod_base = OTHER; note = thymine
  modified_base 6: mod_base = OTHER; note = thymine
  modified_base 10: mod_base = OTHER; note = thymine
  modified_base 12: mod_base = OTHER; note = thymine
  modified_base 17: mod_base = OTHER; note = thymine
  modified_base 19: mod_base = OTHER; note = thymine
  misc_feature 1..21: note = guide sequence
  source 1..21: mol_type = other RNA
gtgcatcagtgtgacatctgc

SEQ ID NO: 23 - RNA, 20 nt, synthetic construct
  modified_base 2: mod_base = OTHER; note = thymine
  modified_base 13: mod_base = OTHER; note = thymine
  modified_base 18: mod_base = OTHER; note = thymine
  misc_feature 1..20: note = guide sequence
  source 1..20: mol_type = other RNA
gtaaacaaagcatagactga

SEQ ID NO: 24 - RNA, 20 nt, synthetic construct
  modified_base 2..3: mod_base = OTHER; note = thymine
  modified_base 13: mod_base = OTHER; note = thymine
  modified_base 18: mod_base = OTHER; note = thymine
  misc_feature 1..20: note = guide sequence
  source 1..20: mol_type = other RNA
gttaacaaagcatagactga

SEQ ID NO: 25 - RNA, 20 nt, synthetic construct
  modified_base 2: mod_base = OTHER; note = thymine
  modified_base 5: mod_base = OTHER; note = thymine
  modified_base 13: mod_base = OTHER; note = thymine
  modified_base 18: mod_base = OTHER; note = thymine
  misc_feature 1..20: note = guide sequence
  source 1..20: mol_type = other RNA
gtaatcaaagcatagactga

SEQ ID NO: 26 - RNA, 19 nt, synthetic construct
  modified_base 2: mod_base = OTHER; note = thymine
  modified_base 12: mod_base = OTHER; note = thymine
  modified_base 17: mod_base = OTHER; note = thymine
  misc_feature 1..19: note = guide sequence
  source 1..19: mol_type = other RNA
gtaacaaagcatagactga

SEQ ID NO: 27 - RNA, 21 nt, synthetic construct
  modified_base 2: mod_base = OTHER; note = thymine
  modified_base 14: mod_base = OTHER; note = thymine
  modified_base 19: mod_base = OTHER; note = thymine
  misc_feature 1..21: note = guide sequence
  source 1..21: mol_type = other RNA
gtgaaacaaagcatagactga

SEQ ID NO: 28 - RNA, 20 nt, synthetic construct
  modified_base 12: mod_base = OTHER; note = thymine
  modified_base 15: mod_base = OTHER; note = thymine
  misc_feature 1..20: note = guide sequence
  source 1..20: mol_type = other RNA
gaagagcagggtcatgaagg

SEQ ID NO: 29 - RNA, 20 nt, synthetic construct
  modified_base 3: mod_base = OTHER; note = thymine
  modified_base 12: mod_base = OTHER; note = thymine
  modified_base 15: mod_base = OTHER; note = thymine
  misc_feature 1..20: note = guide sequence
  source 1..20: mol_type = other RNA
gatgagcagggtcatgaagg

SEQ ID NO: 30 - RNA, 19 nt, synthetic construct
  modified_base 11: mod_base = OTHER; note = thymine
  modified_base 14: mod_base = OTHER; note = thymine
  misc_feature 1..19: note = guide sequence
  source 1..19: mol_type = other RNA
gagagcagggtcatgaagg

SEQ ID NO: 31 - RNA, 21 nt, synthetic construct
  modified_base 13: mod_base = OTHER; note = thymine
  modified_base 16: mod_base = OTHER; note = thymine
  misc_feature 1..21: note = guide sequence
  source 1..21: mol_type = other RNA
gcaagagcagggtcatgaagg

SEQ ID NO: 32 - RNA, 20 nt, synthetic construct
  modified_base 11: mod_base = OTHER; note = thymine
  modified_base 19: mod_base = OTHER; note = thymine
  misc_feature 1..20: note = guide sequence
  source 1..20: mol_type = other RNA
ggagcacacatgcccaggtg

SEQ ID NO: 33 - RNA, 20 nt, synthetic construct
  modified_base 3: mod_base = OTHER; note = thymine
  modified_base 11: mod_base = OTHER; note = thymine
  modified_base 19: mod_base = OTHER; note = thymine
  misc_feature 1..20: note = guide sequence
  source 1..20: mol_type = other RNA
ggtgcacacatgcccaggtg

SEQ ID NO: 34 - RNA, 20 nt, synthetic construct
  modified_base 11: mod_base = OTHER; note = thymine
  modified_base 19: mod_base = OTHER; note = thymine
  misc_feature 1..20: note = guide sequence
  source 1..20: mol_type = other RNA
ggaccacacatgcccaggtg

SEQ ID NO: 35 - RNA, 19 nt, synthetic construct
  modified_base 10: mod_base = OTHER; note = thymine
  modified_base 18: mod_base = OTHER; note = thymine
  misc_feature 1..19: note = guide sequence
  source 1..19: mol_type = other RNA
gagcacacatgcccaggtg

SEQ ID NO: 36 - RNA, 21 nt, synthetic construct
  modified_base 3: mod_base = OTHER; note = thymine
  modified_base 12: mod_base = OTHER; note = thymine
  modified_base 20: mod_base = OTHER; note = thymine
  misc_feature 1..21: note = guide sequence
  source 1..21: mol_type = other RNA
ggtagcacacatgcccaggtg

SEQ ID NO: 37 - RNA, 20 nt, synthetic construct
  modified_base 13: mod_base = OTHER; note = thymine
  modified_base 18: mod_base = OTHER; note = thymine
  misc_feature 1..20: note = guide sequence
  source 1..20: mol_type = other RNA
gaacacaaagcatagactgc

SEQ ID NO: 38 - RNA, 20 nt, synthetic construct
  modified_base 2: mod_base = OTHER; note = thymine
  modified_base 13: mod_base = OTHER; note = thymine
  modified_base 18: mod_base = OTHER; note = thymine
  misc_feature 1..20: note = guide sequence
  source 1..20: mol_type = other RNA
gtacacaaagcatagactgc

SEQ ID NO: 39 - RNA, 20 nt, synthetic construct
  modified_base 13: mod_base = OTHER; note = thymine
  modified_base 18: mod_base = OTHER; note = thymine
  misc_feature 1..20: note = guide sequence
  source 1..20: mol_type = other RNA
gaagacaaagcatagactgc

SEQ ID NO: 40 - RNA, 19 nt, synthetic construct
  modified_base 12: mod_base = OTHER; note = thymine
  modified_base 17: mod_base = OTHER; note = thymine
  misc_feature 1..19: note = guide sequence
  source 1..19: mol_type = other RNA
gaaacaaagcatagactgc

SEQ ID NO: 41 - RNA, 21 nt, synthetic construct
  modified_base 3: mod_base = OTHER; note = thymine
  modified_base 14: mod_base = OTHER; note = thymine
  modified_base 19: mod_base = OTHER; note = thymine
  misc_feature 1..21: note = guide sequence
  source 1..21: mol_type = other RNA
gatacacaaagcatagactgc

SEQ ID NO: 42 - RNA, 20 nt, synthetic construct
  modified_base 9: mod_base = OTHER; note = thymine
  modified_base 13: mod_base = OTHER; note = thymine
  modified_base 17..18: mod_base = OTHER; note = thymine
  misc_feature 1..20: note = guide sequence
  source 1..20: mol_type = other RNA
gaacacaatgcatagattgc

SEQ ID NO: 43 - RNA, 20 nt, synthetic construct
  modified_base 3: mod_base = OTHER; note = thymine
  modified_base 9: mod_base = OTHER; note = thymine
  modified_base 13: mod_base = OTHER; note = thymine
  modified_base 17..18: mod_base = OTHER; note = thymine
  misc_feature 1..20: note = guide sequence
  source 1..20: mol_type = other RNA
gatcacaatgcatagattgc

SEQ ID NO: 44 - RNA, 20 nt, synthetic construct
  modified_base 5: mod_base = OTHER; note = thymine
  modified_base 9: mod_base = OTHER; note = thymine
  modified_base 13: mod_base = OTHER; note = thymine
  modified_base 17..18: mod_base = OTHER; note = thymine
  misc_feature 1..20: note = guide sequence
  source 1..20: mol_type = other RNA
gaactcaatgcatagattgc

SEQ ID NO: 45 - RNA, 19 nt, synthetic construct
  modified_base 8: mod_base = OTHER; note = thymine
  modified_base 12: mod_base = OTHER; note = thymine
  modified_base 16..17: mod_base = OTHER; note = thymine
  misc_feature 1..19: note = guide sequence
  source 1..19: mol_type = other RNA
gacacaatgcatagattgc

SEQ ID NO: 46 - RNA, 21 nt, synthetic construct
  modified_base 10: mod_base = OTHER; note = thymine
  modified_base 14: mod_base = OTHER; note = thymine
  modified_base 18..19: mod_base = OTHER; note = thymine
  misc_feature 1..21: note = guide sequence
  source 1..21: mol_type = other RNA
gacacacaatgcatagattgc

SEQ ID NO: 47 - RNA, 20 nt, synthetic construct
  modified_base 1: mod_base = OTHER; note = thymine
  modified_base 9..10: mod_base = OTHER; note = thymine
  modified_base 13: mod_base = OTHER; note = thymine
  misc_feature 1..20: note = guide sequence
  source 1..20: mol_type = other RNA
tcacacacttgatcacagag

SEQ ID NO: 48 - RNA, 20 nt, synthetic construct
  modified_base 1: mod_base = OTHER; note = thymine
  modified_base 9..10: mod_base = OTHER; note = thymine
  modified_base 13: mod_base = OTHER; note = thymine
  misc_feature 1..20: note = guide sequence
  source 1..20: mol_type = other RNA
tgacacacttgatcacagag

SEQ ID NO: 49 - RNA, 20 nt, synthetic construct
  modified_base 1: mod_base = OTHER; note = thymine
  modified_base 4: mod_base = OTHER; note = thymine
  modified_base 9..10: mod_base = OTHER; note = thymine
  modified_base 13: mod_base = OTHER; note = thymine
  misc_feature 1..20: note = guide sequence
  source 1..20: mol_type = other RNA
tcatacacttgatcacagag

SEQ ID NO: 50 - RNA, 19 nt, synthetic construct
  modified_base 1: mod_base = OTHER; note = thymine
  modified_base 8..9: mod_base = OTHER; note = thymine
  modified_base 12: mod_base = OTHER; note = thymine
  misc_feature 1..19: note = guide sequence
  source 1..19: mol_type = other RNA
tacacacttgatcacagag

SEQ ID NO: 51 - RNA, 21 nt, synthetic construct
  modified_base 1: mod_base = OTHER; note = thymine
  modified_base 10..11: mod_base = OTHER; note = thymine
  modified_base 14: mod_base = OTHER; note = thymine
  misc_feature 1..21: note = guide sequence
  source 1..21: mol_type = other RNA
tcaacacacttgatcacagag

SEQ ID NO: 52 - RNA, 20 nt, synthetic construct
  modified_base 18: mod_base = OTHER; note = thymine
  misc_feature 1..20: note = guide sequence
  source 1..20: mol_type = other RNA
gaagaaggagacaccgatga

SEQ ID NO: 53 - RNA, 20 nt, synthetic construct
  modified_base 18: mod_base = OTHER; note = thymine
  misc_feature 1..20: note = guide sequence
  source 1..20: mol_type = other RNA
gacgaaggagacaccgatga

SEQ ID NO: 54 - RNA, 19 nt, synthetic construct
  modified_base 17: mod_base = OTHER; note = thymine
  misc_feature 1..19: note = guide sequence
  source 1..19: mol_type = other RNA
gagaaggagacaccgatga

SEQ ID NO: 55 - RNA, 21 nt, synthetic construct
  modified_base 19: mod_base = OTHER; note = thymine
  misc_feature 1..21: note = guide sequence
  source 1..21: mol_type = other RNA
gcaagaaggagacaccgatga

SEQ ID NO: 56 - RNA, 20 nt, synthetic construct
  modified_base 1: mod_base = OTHER; note = thymine
  modified_base 15: mod_base = OTHER; note = thymine
  modified_base 17: mod_base = OTHER; note = thymine
  misc_feature 1..20: note = guide sequence
  source 1..20: mol_type = other RNA
tgacacaggagacctgtcac

SEQ ID NO: 57 - RNA, 20 nt, synthetic construct
  modified_base 1: mod_base = OTHER; note = thymine
  modified_base 6: mod_base = OTHER; note = thymine
  modified_base 15: mod_base = OTHER; note = thymine
  modified_base 17: mod_base = OTHER; note = thymine
  misc_feature 1..20: note = guide sequence
  source 1..20: mol_type = other RNA
tgacataggagacctgtcac

SEQ ID NO: 58 - RNA, 19 nt, synthetic construct
  modified_base 1: mod_base = OTHER; note = thymine
  modified_base 14: mod_base = OTHER; note = thymine
  modified_base 16: mod_base = OTHER; note = thymine
  misc_feature 1..19: note = guide sequence
  source 1..19: mol_type = other RNA
tgaacaggagacctgtcac

SEQ ID NO: 59 - RNA, 21 nt, synthetic construct
  modified_base 1: mod_base = OTHER; note = thymine
  modified_base 16: mod_base = OTHER; note = thymine
  modified_base 18: mod_base = OTHER; note = thymine
  misc_feature 1..21: note = guide sequence
  source 1..21: mol_type = other RNA
tgaacacaggagacctgtcac

SEQ ID NO: 60 - RNA, 20 nt, synthetic construct
  modified_base 1: mod_base = OTHER; note = thymine
  modified_base 10..12: mod_base = OTHER; note = thymine
  misc_feature 1..20: note = guide sequence
  source 1..20: mol_type = other RNA
tgggaagggtttccagagga

SEQ ID NO: 61 - RNA, 20 nt, synthetic construct
  modified_base 1: mod_base = OTHER; note = thymine
  modified_base 10..12: mod_base = OTHER; note = thymine
  misc_feature 1..20: note = guide sequence
  source 1..20: mol_type = other RNA
taggaagggtttccagagga

SEQ ID NO: 62 - RNA, 19 nt, synthetic construct
  modified_base 1: mod_base = OTHER; note = thymine
  modified_base 9..11: mod_base = OTHER; note = thymine
  misc_feature 1..19: note = guide sequence
  source 1..19: mol_type = other RNA
tggaagggtttccagagga

SEQ ID NO: 63 - RNA, 21 nt, synthetic construct
  modified_base 1: mod_base = OTHER; note = thymine
  modified_base 3: mod_base = OTHER; note = thymine
  modified_base 11..13: mod_base = OTHER; note = thymine
  misc_feature 1..21: note = guide sequence
  source 1..21: mol_type = other RNA
tgtggaagggtttccagagga

SEQ ID NO: 64 - RNA, 20 nt, synthetic construct
  modified_base 2: mod_base = OTHER; note = thymine
  modified_base 13: mod_base = OTHER; note = thymine
  modified_base 15: mod_base = OTHER; note = thymine
  misc_feature 1..20: note = guide sequence
  source 1..20: mol_type = other RNA
atacaggagacctgtcacag

SEQ ID NO: 65 - RNA, 19 nt, synthetic construct
  modified_base 12: mod_base = OTHER; note = thymine
  modified_base 14: mod_base = OTHER; note = thymine
  misc_feature 1..19: note = guide sequence
  source 1..19: mol_type = other RNA
aacaggagacctgtcacag

SEQ ID NO: 66 - RNA, 21 nt, synthetic construct
  modified_base 14: mod_base = OTHER; note = thymine
  modified_base 16: mod_base = OTHER; note = thymine
  misc_feature 1..21: note = guide sequence
  source 1..21: mol_type = other RNA
acgacaggagacctgtcacag

SEQ ID NO: 67 - RNA, 20 nt, synthetic construct
  modified_base 8: mod_base = OTHER; note = thymine
  modified_base 16: mod_base = OTHER; note = thymine
  modified_base 18: mod_base = OTHER; note = thymine
  misc_feature 1..20: note = guide sequence
  source 1..20: mol_type = other RNA
gcacacatgcccaggtgtgg

SEQ ID NO: 68 - RNA, 20 nt, synthetic construct
  modified_base 8: mod_base = OTHER; note = thymine
  modified_base 16: mod_base = OTHER; note = thymine
  modified_base 18: mod_base = OTHER; note = thymine
  misc_feature 1..20: note = guide sequence
  source 1..20: mol_type = other RNA
ggacacatgcccaggtgtgg

SEQ ID NO: 69 - RNA, 19 nt, synthetic construct
  modified_base 7: mod_base = OTHER; note = thymine
  modified_base 15: mod_base = OTHER; note = thymine
  modified_base 17: mod_base = OTHER; note = thymine
  misc_feature 1..19: note = guide sequence
  source 1..19: mol_type = other RNA
gccacatgcccaggtgtgg

SEQ ID NO: 70 - RNA, 21 nt, synthetic construct
  modified_base 2: mod_base = OTHER; note = thymine
  modified_base 9: mod_base = OTHER; note = thymine
  modified_base 17: mod_base = OTHER; note = thymine
  modified_base 19: mod_base = OTHER; note = thymine
  misc_feature 1..21: note = guide sequence
  source 1..21: mol_type = other RNA
gtcacacatgcccaggtgtgg

SEQ ID NO: 71 - RNA, 22 nt, synthetic construct
  modified_base 16..17: mod_base = OTHER; note = thymine
  modified_base 20..21: mod_base = OTHER; note = thymine
  misc_feature 1..22: note = guide sequence
  source 1..22: mol_type = other RNA
ggagagaaagagaagttgattg

SEQ ID NO: 72 - RNA, 22 nt, synthetic construct
  modified_base 16..17: mod_base = OTHER; note = thymine
  modified_base 20..21: mod_base = OTHER; note = thymine
  misc_feature 1..22: note = guide sequence
  source 1..22: mol_type = other RNA
ggcgagaaagagaagttgattg

SEQ ID NO: 73 - RNA, 22 nt, synthetic construct
  modified_base 2..3: mod_base = OTHER; note = thymine
  modified_base 16..17: mod_base = OTHER; note = thymine
  modified_base 20..21: mod_base = OTHER; note = thymine
  misc_feature 1..22: note = guide sequence
  source 1..22: mol_type = other RNA
gttgagaaagagaagttgattg

SEQ ID NO: 74 - RNA, 20 nt, synthetic construct
  modified_base 14..15: mod_base = OTHER; note = thymine
  modified_base 18..19: mod_base = OTHER; note = thymine
  misc_feature 1..20: note = guide sequence
  source 1..20: mol_type = other RNA
ggagaaagagaagttgattg

SEQ ID NO: 75 - RNA, 23 nt, synthetic construct
  modified_base 17..18: mod_base = OTHER; note = thymine
  modified_base 21..22: mod_base = OTHER; note = thymine
  misc_feature 1..23: note = guide sequence
  source 1..23: mol_type = other RNA
gggagagaaagagaagttgattg

SEQ ID NO: 76 - RNA, 22 nt, synthetic construct
  modified_base 3: mod_base = OTHER; note = thymine
  modified_base 5..6: mod_base = OTHER; note = thymine
  modified_base 10: mod_base = OTHER; note = thymine
  misc_feature 1..22: note = guide sequence
  source 1..22: mol_type = other RNA
gctgttgcatgaggaaagggac

SEQ ID NO: 77 - RNA, 22 nt, synthetic construct
  modified_base 2: mod_base = OTHER; note = thymine
  modified_base 5..6: mod_base = OTHER; note = thymine
  modified_base 10: mod_base = OTHER; note = thymine
  misc_feature 1..22: note = guide sequence
  source 1..22: mol_type = other RNA
gtagttgcatgaggaaagggac

SEQ ID NO: 78 - RNA, 22 nt, synthetic construct
  modified_base 3: mod_base = OTHER; note = thymine
  modified_base 5..6: mod_base = OTHER; note = thymine
  modified_base 10: mod_base = OTHER; note = thymine
  misc_feature 1..22: note = guide sequence
  source 1..22: mol_type = other RNA
ggtgttgcatgaggaaagggac

SEQ ID NO: 79 - RNA, 20 nt, synthetic construct
  modified_base 3..4: mod_base = OTHER; note = thymine
  modified_base 8: mod_base = OTHER; note = thymine
  misc_feature 1..20: note = guide sequence
  source 1..20: mol_type = other RNA
gcttgcatgaggaaagggac

SEQ ID NO: 80 - RNA, 24 nt, synthetic construct
  modified_base 3: mod_base = OTHER; note = thymine
  modified_base 5: mod_base = OTHER; note = thymine
  modified_base 7..8: mod_base = OTHER; note = thymine
  modified_base 12: mod_base = OTHER; note = thymine
  misc_feature 1..24: note = guide sequence
  source 1..24: mol_type = other RNA
gctatgttgcatgaggaaagggac

SEQ ID NO: 81 - RNA, 76 nt, synthetic construct
  modified_base 2..5: mod_base = OTHER; note = thymine
  modified_base 11: mod_base = OTHER; note = thymine
  modified_base 17: mod_base = OTHER; note = thymine
  modified_base 24..25: mod_base = OTHER; note = thymine
  modified_base 30: mod_base = OTHER; note = thymine
  modified_base 36: mod_base = OTHER; note = thymine
  modified_base 39: mod_base = OTHER; note = thymine
  modified_base 43..44: mod_base = OTHER; note = thymine
  modified_base 46: mod_base = OTHER; note = thymine
  modified_base 51..52: mod_base = OTHER; note = thymine
  modified_base 60: mod_base = OTHER; note = thymine
  modified_base 70: mod_base = OTHER; note = thymine
  modified_base 74: mod_base = OTHER; note = thymine
  misc_feature 1..76: note = guide sequence
  source 1..76: mol_type = other RNA
gttttagagctagaaatagcaagttaaaataaggctagtccgttatcaacttgaaaaagtggcaccgagtcggtgc

SEQ ID NO: 82 - RNA, 76 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 49: mod_base = OTHER; note = thymine
  modified_base 51..53: mod_base = OTHER; note = thymine
  modified_base 55: mod_base = OTHER; note = thymine
  modified_base 57: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  misc_feature 1..76: note = guide sequence
  source 1..76: mol_type = other RNA
gttttagtactctggaaacagaatctactaaaacaaggcaaaatgccgtgtttatctcgtcaacttgttggcgaga

SEQ ID NO: 83 - protein, 166 aa, synthetic construct
  REGION 1..166: note = TadA mutant sequence
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 84 - protein, 166 aa, synthetic construct
  REGION 1..166: note = TadA mutant sequence
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 85 - protein, 1368 aa, synthetic construct
  REGION 1..1368: note = SpdCas9 sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 86 - protein, 1368 aa, synthetic construct
  REGION 1..1368: note = SpnCas9 sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 87 - protein, 1053 aa, synthetic construct
  REGION 1..1053: note = SadCas9 sequence
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 88 - protein, 1053 aa, synthetic construct
  REGION 1..1053: note = SanCas9 sequence
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 89 - protein, 1228 aa, synthetic construct
  REGION 1..1228: note = LbdCpf1 sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVAFLISPVKNSDGIFYDSRNYEAQENAILPKNADANGAYNIARKVLWAIGQFKKAEDEKLDKVKIAISNKEWLEYAQTSVKH